(19) United States
(12) Patent Application Publication
(10) Pub. No.: US 2025/0242023 A1
(43) Pub. Date: Jul. 31, 2025

(54) GENETICALLY ENGINEERED T CELL RECEPTORS

(71) Applicant: REGENTS OF THE UNIVERSITY OF MINNESOTA, Minneapolis, MN (US)

(72) Inventors: Ingunn Stromnes, Minneapolis, MN (US); Branden S. Moriarity, Minneapolis, MN (US); Beau r. Webber, Minneapolis, MN (US)

(21) Appl. No.: 18/703,440

(22) PCT Filed: Oct. 24, 2022
(86) PCT No.: PCT/US2022/078591

§ 371 Date: Apr. 22, 2024

Related U.S. Application Data
(60) Provisional application No. 63/270,795, filed on Oct. 22, 2021.

(51) Int. Cl. A61K 40/32; A01K 67/0278; A61K 40/11; A61K 40/42; C07K 14/725; C12N 9/22; C12N 15/11; C12N 15/90

(52) U.S. Cl. CPC A61K 40/32; A01K 67/0278; A61K 40/11; A61K 40/4202; C07K 14/7051; C12N 9/226; C12N 15/11; C12N 15/907; A01K 2217/072; A01K 2227/105; C12N 2310/20

(57) ABSTRACT

The present disclosure relates, in general, to methods for generating engineered antigen-specific T cell receptors, cells and non-human animals comprising such engineered T cell receptors and methods of making engineered T cell receptors. The engineered T cell receptors can be specific for cancer or immunology targets, such as mesothelin, and are useful in developing therapies for cancer, autoimmune diseases, infectious diseases and other conditions or disorders.

### CROSS-REFERENCE TO RELATED APPLICATIONS

The present application claims the priority benefit of U.S. Provisional Patent Application No. 63/270,795, filed Oct. 22, 2021, herein incorporated by reference in its entirety.

### FIELD OF THE DISCLOSURE

The present disclosure relates, in general, to engineered T cell receptors, cells and non-human animals comprising such engineered T cell receptors and methods of making engineered T cell receptors.

### BACKGROUND

The understanding of antigen-specific T cell responses at steady state and in disease have benefited from the use of T cell receptor (TCR) transgenic mice.

Mesothelin (MsIn) is a self-antigen overexpressed in many malignancies including pancreatic (1-3), ovarian (4), lung (5), and breast (6) cancer. MsIn-specific T cells are detected in cancer patients following vaccination demonstrating its immunogenicity in humans (7). MsIn is expressed at low levels in the pleura, peritoneum and pericardium in mice and humans and MsIn^{-/-} mice lack a discernable phenotype (8). Thus, MsIn is a promising target for cancer therapy (9).

T cell receptor (TCR) transgenic mice have served as the foundation for seminal studies describing T cell development and function. TCR transgenic mouse strains have contributed greatly to our understanding of T cell development and differentiation. Historically, transgenic TCRs are randomly integrated and expression is driven be heterologous promoter fragments including MHC class I, as in P14 T cells (10), CD2 (33, 34), or endogenous TCR promoter and regulatory flanking regions (35, 36). Such models require substantial time to generate, and random genomic integration and non-physiologic promoters may impact T cell functionality. Transgenic TCRs are abnormally expressed in immature double negative thymocytes, the stage in which endogenous Tcrb genes typically undergo rearrangement, thereby interfering with endogenous TCR rearrangement and resulting in the transgenic TCR expressed on most T cells (35, 37). it is well appreciated that transgenic T cells can also express endogenous TCRs (38). To avoid endogenous TCR expression, transgenic mice can be bred to a Rag^{-/-} or TCRα^{-/-} background to ensure that only the transgenic TCR is expressed.

However, random vector integration and non-physiological TCR expression can impair T cell fidelity. A panel of murine and human T cell receptors (TCRs) specific to MsIn epitopes with a range of affinities have previously been cloned (2). It was shown that these engineered T cells preferentially accumulated in primary tumors and metastasis, recognized tumor antigen, caused objective responses, and significantly prolonged animal survival (2, 12). Despite observed therapeutic efficacy, certain T cell clones were rendered dysfunctional in the tumor microenvironment (TME) (2, 12).

### SUMMARY

The present disclosure provides improved methods for generating genetically engineered T cell receptors specific for a particular antigenic target of interest. The disclosure provides a more efficient method for integrating exogenous T cell receptor into an endogenous locus in order to construct a modified T cell receptor, and expression thereof in a cell or animal.

In various embodiments, the disclosure provides a genetically engineered non-human animal comprising i) a TCR exchanged (TRex) T cell receptor locus expressing a T cell receptor specific for mesothelin, wherein the non-human animal also expresses less than 15% endogenous T cell receptor on TCR α or β expressing cells; and ii) an inactivated mesothelin gene.

In various embodiments, the TCR exchange is introduced in the T cell receptor alpha (Trac) locus. In various embodiments, the TCR exchange comprises nuclease-dependent cleavage system disruption of the Trac locus and introduction of a polynucleotide encoding the T cell receptor specific for mesothelin. In certain embodiments, the nuclease dependent cleavage system comprises a CRISPR/Cas system, a Cas-CLOVER system, a zinc-finger nuclease (ZFN) system, a transcription activator like effector nuclease (TALEN) system, or a meganuclease system.

In various embodiments, the nuclease dependent cleavage system is a CRISPR/Cas system. In various embodiments, the CRISPR/Cas system comprises Cas9, Cas12a, Cas13a or Cas13b.

In various embodiments, the polynucleotide encoding the T cell receptor specific for mesothelin is expressed on a viral vector, optionally an AAV vector. In various embodiments, the AAV is AAV6, AAV1 or AAV-DJ.

In one embodiment, the animal expresses high affinity mesothelin-specific T cells. In one embodiment, wherein the animal expresses low affinity mesothelin-specific T cells.

In various embodiments, the T cells expressing the mesothelin-specific TCR are CD4+ T cells or CD8+ T cells.

In certain embodiments, the animal is a mouse. In various embodiments, the mouse is on a C57Bl/6 background or NOD background.

In various embodiments, the high affinity mesothelin-specific T cells express a 1045 TCR. In various embodiments, the low affinity mesothelin-specific T cells express a 7431 TCR.

In various embodiments, the mesothelin gene is disrupted in exon 4 of the mesothelin gene.

In various embodiments, the genetically engineered animal is homozygous for the donor TCR or heterozygous for the donor TCR. In various embodiments, the genetically engineered animal is homozygous for the mesothelin knockout.

Further contemplated is a T cell expressing a T cell receptor specific for mesothelin isolated from a genetically engineered non-human animal described herein. In various embodiments, the T cell is a CD4+ T cell or CD8+ T cell. In various embodiments, the T cell is an effector T cell or a memory T cell. In various embodiments, the T cell is CD44^{low}/CD62L⁺, CD44highCD26L− or CD44highCD62L+.

In another embodiment, the disclosure provides a method of measuring effects of T cells having TCR exchanged (TRex) T cell receptor locus expressing a T cell receptor specific for mesothelin comprising contacting the T cell with mesothelin presented in MHC and measuring the effects on the T cell. In various embodiments, the effects include stimulation of cytokine production, modulation of cell surface marker phenotype, change in activation phenotype, modulation of number of regulatory T cells induced, or cytotoxicity phenotype, replicating endogenous TCR gene regulation following antigen encounter, and eliminating endogenous TRAC expression.

In various embodiments, the mesothelin is expressed by a cancer cell. In various embodiments, the cancer cell is a pancreatic, ovarian, lung, or breast cancer cell.

In various embodiments, the disclosure provides a method of making an engineered T cell receptor comprising a T cell receptor exchanged (Trex) locus, the method comprising: i) expressing a T cell receptor gene comprising a T cell receptor alpha (Trac) locus on a plasmid or vector containing homologous sequence to the murine Trac locus; ii) inserting a donor TCR polynucleotide sequence into the T cell receptor alpha (Trac) locus of the T cell receptor gene using a nuclease-dependent cleavage system comprising Trac-specific targeting molecules specific to Trac exon 1 or directly 5′ to Trac exon 1 complexed to a ribonucleoprotein (RNP); and, iii) expressing the engineered TCR from the plasmid or vector.

In certain embodiments, the nuclease dependent cleavage system comprises a CRISPR/Cas system, a Cas-CLOVER system, a zinc-finger nuclease (ZFN) system, a transcription activator like effector nuclease (TALEN) system, or a meganuclease system.

Also provided is a method of making an engineered T cell receptor comprising a T cell receptor exchanged (Trex) locus, the method comprising i) expressing a T cell receptor gene comprising a T cell receptor alpha (Trac) locus on a plasmid or vector containing homologous sequence to the murine Trac locus; ii) inserting a donor TCR polynucleotide sequence into the T cell receptor alpha (Trac) locus of the T cell receptor gene using a CRISPR/Cas gene editing system comprising Trac-specific guide RNAs (gRNAs) specific to Trac exon 1 or directly 5′ to Trac exon 1 complexed to Cas ribonucleoprotein (RNP); and, iii) expressing the engineered TCR from the plasmid or vector. In various embodiments, the expression is from and endogenous locus.

In various embodiments, the T cell receptor is expressed in a CD4+ T cell or CD8+ T cell. In various embodiments, the T cell is an effector T cell or a memory T cell.

Further provided is a method of making a genetically engineered non-human animal comprising a T cell receptor exchanged (Trex) locus, the method comprising i) expressing a donor T cell receptor polynucleotide specific for a target antigen on a plasmid or vector; and ii) inserting the donor TCR polynucleotide of i) into T cell receptor alpha (Trac) locus of a T cell receptor gene in a non-human animal zygote using a CRISPR/Cas gene editing system comprising Trac-specific guide RNAs (gRNAs) specific to Trac exon 1 (Trac gRNA 1) or directly 5′ to Trac exon 1 (Trac gRNA 2) complexed to a Cas ribonucleoprotein (RNP).

In various embodiments, the donor TCR sequences comprise a TCR3 variable (V), TCRβ Constant (C) and TCRα V sequences. In various embodiments, the exogenous TCRβ, TCRα, and endogenous Trac sequences are linked by self-cleaving 2A element.

In various embodiments, the guide RNAs are nucleofected into activated splenic polyclonal T cells.

In various embodiments, the donor TCR sequence is encoded in an AAV vector. In various embodiments, the donor TCR sequence is flanked by approximately 250 to 1000 bp homology arms (HA) encoding endogenous murine Trac sequences and cloned into an AAV vector. In various embodiments, the AAV is AAV6, AAV1 or AAV-DJ.

In various embodiments, CRISPR/Cas9 initiates a double-strand DNA break directly upstream of Trac or in exon 1.

In various embodiments, T cells expressing a TRex TCR specific for the target antigen upon activation upregulate CD44 and maintain CD62L levels, downregulate TCR, and/or minimally express PD-1.

In various embodiments, rAAV expressing the TRex locus is administered to embryos at a final concentration of between 1.0×10⁸ GC/μl and 3×10⁸ GC/μl.

In various embodiments, the method further comprises inactivating a gene encoding the target antigen of interest in the non-human animal. In various embodiments, the gene encoding the target antigen is inactivated using a nuclease-dependent cleavage system.

In various embodiments, 80% or more of CD4 and/or CD8 T cells in the genetically engineered non-human animal express an engineered TCR.

In various embodiments, the T cells expressing the Trex TCR are not tolerized to the target antigen. In various embodiments, T cells expressing the Trex TCR upon activation upregulate CD44 and maintain CD62L levels, downregulate TCR, and/or minimally express PD-1.

In various embodiments, the target antigen is a cancer antigen, autoimmune antigen, or foreign antigen. In various embodiments, the target antigen is mesothelin.

In another embodiment, the disclosure provides a genetically engineered non-human animal comprising i) a TCR exchanged (TRex) T cell receptor locus expressing a T cell receptor specific for a protein of interest, wherein the non-human animal also expresses less than 15% endogenous T cell receptor on TCR α or β expressing cells; and ii) an inactivated gene of the protein of interest.

In various embodiments, in the genetically engineered non-human animal the TCR exchange is introduced in the T cell receptor alpha (Trac) locus. In various embodiments, the TCR exchange comprises nuclease-dependent cleavage system disruption of the Trac locus and introduction of a polynucleotide encoding the T cell receptor specific for the protein of interest.

In various embodiments, the nuclease dependent cleavage system comprises a CRISPR/Cas system, a Cas-CLOVER system, a zinc-finger nuclease (ZFN) system, a transcription activator like effector nuclease (TALEN) system, or a meganuclease system. In various embodiments, the nuclease dependent cleavage system comprises a CRISPR/Cas system. In various embodiments, the CRISPR/Cas system comprises Cas9, Cas12a, Cas13a or Cas13b.

In various embodiments, the polynucleotide encoding the T cell receptor specific for the protein of interest is expressed on a viral vector, optionally an AAV vector.

In various embodiments, the genetically engineered non-human animal expresses high affinity antigen-specific T cells. In various embodiments, the genetically engineered non-human animal expresses low affinity antigen-specific T cells.

In various embodiments, in the genetically engineered non-human animal the T cells expressing the antigen-specific TCR are CD4+ T cells or CD8+ T cells.

In various embodiments, the genetically engineered non-human animal is a mouse.

In various embodiments, the genetically engineered non-human animal is homozygous for the donor TCR or heterozygous for the donor TCR. In various embodiments, in the genetically engineered non-human animal is homozygous for the protein knockout.

Also contemplated herein is a T cell expressing a T cell receptor specific for a protein of interest isolated from a genetically engineered non-human animal as described herein.

Further aspects and advantages will be apparent to those of ordinary skill in the art from a review of the following detailed description, taken in conjunction with the drawings. While the compositions, articles, and methods are susceptible of embodiments in various forms, the description hereafter includes specific embodiments with the understanding that the disclosure is illustrative, and is not intended to limit the invention to the specific embodiments described herein. For the compositions, articles, and methods described herein, optional features, including but not limited to components, compositional ranges thereof, substituents, conditions, and steps, are contemplated to be selected from the various aspects, embodiments, and examples provided herein.

### BRIEF DESCRIPTION OF THE DRAWINGS

FIG. 1A-1H. TCR replacement with MsIn TCRs using CRISPR/Cas9 and rAAV in primary murine T cells. FIG. 1A) Schematic of TCR targeting approach. Donor DNA is flanked by homology arms (HA) and encoded by rAAV. FIG. 1B) Protocol for testing TCR replacement using CRISPR/Cas9 and rAAV. FIG. 1C) Efficiency of Trac gRNAs was measured by loss of TCRP staining and flow cytometry. FIG. 1D) Va2 expression in activated P14 T cells on day 3 post nucleofection with Trac1 or Trac2 gRNAs complexed to Cas9 RNP.

FIG. 1E) Representation of Trac1 and Trac2 gRNAs on murine chromosome 14. FIG. 1F) Representative flow cytometry plots of donor TCR expression in murine T cells was determined by staining for Vβ9. MOI, multiplicity of infection. FIG. 1G) Quantification of Vβ9 on CD4 and CD8 T cells at the indicated AAV MOIs. Data are mean±S.E.M. and pooled from 3 independent experiments. FIG. 1H) Representative flow cytometric plots of engineered T cell expansion 5 days post a second in vitro stimulation with MsIn 406-414-pulsed irradiated APCs and cytokines.

FIG. 2A-2K. Targeting MsIn TCRs into Trac promotes engineered T cell function and obviates Treg expansion. FIG. 2A) Overview of retroviral transduction (RV) of MsIn TCRs in P14 T cells. FIG. 2B) Overview of CRISPR/Cas9+rAAV TCR knockin (KI) approach in polyclonal T cells. FIG. 2C) Representative plots of Vβ9 gated on CD4 T cells 5 days after either RV or KI. FIG. 2D) Representative plots of Vβ9 gated on CD8 T cells 5 days after either RV or KI. FIG. 2E) Quantification of C and D. FIG. 2F) Frequency of CD4 or CD8 T cells that are Ki67+Vβ9+ on day 5 post RV or KI. FIG. 2G) Frequency of CD4 or CD8 T cells that are Foxp3+Vβ9+ on day 5 post RV or KI. FIG. 2H) Representative plots gated on live CD4+Vβ9+ T cells. FIG. 2I) Representative plots gated on live CD8+Vβ9+ T cells. FIG. 2J) Representative plots gated on CD4+Vβ9+ T cells. Intracellular cytokine staining was assessed after the second (Stim 2) and third (Stim 3) restimulation in vitro with MsIn peptide-pulsed irradiated syngeneic splenocytes and IL-2. FIG. 2K) Representative flow cytometric plots gated on CD8+Vβ9+ T cells. Intracellular cytokine staining was assessed after the second (Stim 2) and third (Stim 3) restimulation in vitro with MsIn peptide-pulsed irradiated syngeneic splenocytes and IL-2. For panels, E-G, data are mean±S.E.M. n=3-6 mice per group. *p<0.05, **p<0.005, and ***p<0.0005. One-way ANOVA with a Tukey's posttest.

FIG. 3A-3H. Highly efficient TCR replacement and MsIn loss in murine zygotes. FIG. 3A) Simplified schematic of the 2 MsIn gRNAs tested (top panel) and Sequence and target sites of gRNAs specific to murine Trac or MsIn. FIG. 3B) EL4 cells were targeted with Trac gRNA complexed with Cas9 RNP or with a combination of Trac gRNA and MsIn gRNA complexed with Cas9, followed by rAAV-1045 or rAAV-7431. MsIn TCR expression was determined by Vβ9 staining. FIG. 3C) Junction PCR design. 5′ Forward primers are located within the engineered locus and in an intron region upstream of the endogenous Trac locus. 3′Reverse primer was located in Trac. FIG. 3D) Representative plots of homozygous WT or 1045 heterozygous KI blood gated on total live (left) or T cell subsets (middle, right) and analyzed for Vβ9. FIG. 3E) Frequency of circulating T cell subsets and Vβ9+ T cell subsets cells in 1045+ pups as determined by PCR. Each dot is an independent mouse. n=5. FIG. 3F) CD4:CD8 ratio in 1045 and 7431 TCR knock-in mice. FIG. 3G) Representative Vβ9 staining from WT, 7431 heterozygous (Het #9 and #13 from I), and 7431 homozygous (Hom #3 from I) blood gated on total circulating mononuclear cells (left) or T cell subsets (middle, right). FIG. 3H) Frequency of circulating T cell subsets and Vβ9+ T cell subsets cells in 7431+ pups. Each dot is an independent mouse. n=13.

FIGS. 4A-4L. High affinity MsIn-specific T cells undergo central tolerance in a MsIn dose dependent manner. FIG. 4A) Thymus weight in grams (g). Data are mean±S.E.M. Each dot is an independent mouse. FIG. 4B) Representative plots gated on live CD45+B220− thymocytes. FIG. 4C) Frequency double negative (DN), double positive (DP), CD4 single positive (CD4 SP) and CD8 SP among CD45+B220− thymocytes. n=4-6 mice per group. Each dot is an independent mouse. Data are mean±S.E.M. *p<0.05, **p<0.005. Anova with a Tukey's posttest. FIG. 4D) Cell number per thymus. Each dot is an independent mouse. Data are mean±S.E.M. n=4-6 mice per group. *p<0.05, **p<0.005. Anova with a Tukey's posttest. FIG. 4E) Representative plots of Vβ9 and CD24 gated on 4 thymocyte developmental stages. Numbers in plots indicate the frequency of Vβ9+ cells. FIG. 4F) Vβ9+ cell frequency among the indicated thymocyte developmental stage. Each dot is an independent mouse. Data are mean±S.E.M. *p<0.05, **p<0.005, ***p<0.0005, ****p<0.0001. Anova with a Tukey's posttest. FIG. 4G) Number of Vβ9+ cells per thymus in the indicated developmental stage. Each dot is an independent mouse. Data are mean±S.E.M. *p<0.05. Anova with a Tukey's posttest. FIG. 4H) Representative Vβ9 histogram overlays gated on DN1-DN4 stages. FIG. 4I) Thymus weight. Data are mean±S.E.M. Each dot is an independent mouse. n=5-10 mice per group. FIG. 4J) Representative plots gated on live CD45+B220− thymocytes. FIG. 4K) Frequency of cells in each thymocyte stage. Data are mean±S.E.M. Each dot is an independent mouse. n=5-10 mice per group. p<0.05, ****p<0.0001. Student's T test. FIG. 4L) Vβ9 frequency among CD8 SP and CD4 SP cells (top) and representative histograms (bottom). Quantified data are mean±S.E.M. n=5-10 mice per group. *p<0.05, ****p<0.0005. Student's T test.

FIG. 5A-5F. 1045 T cells mature in MsIn^{-/-} and MsIn^{+/+} animals and respond to specific antigen. FIG. 5A) Representative plots gated on splenic CD8 T cells isolated from WT, 1045^{+/-} MsIn^{+/-}, 1045^{+/-} MsIn^{-/-}, and 1045^{+/-} MsIn^{-/-} mice. n=3-4 mice per group. FIG. 5B) Representative plots gated on splenic CD4 T cells isolated from WT, 1045^{+/-} MsIn^{+/-}, 1045^{+/-} MsIn^{-/-}, and 1045^{+/-} MsIn^{-/-} mice. n=3-4 mice per group. FIG. 5C) Frequency of CD8 T cells that express Vβ9 (left) and CD8+Vβ9+ T cells that express CD44 or CD62L. FIG. 5D) Frequency of CD4 T cells that express Vβ9 (left) and CD4+Vβ9+ T cells that express CD44 or CD62L. Quantified data from representative activation and proliferation (CTV) plots are gated on CD8+Vβ9+(FIG. 5E) or CD4+Vβ9+(FIG. 5F) T cells on day 3±activation with MsIn₄₀₆₋₄₁₄ peptide or anti-CD3+ anti-CD28. Data are mean±S.E.M. n=3-4 mice per group. *p<0.05, **p<0.005, and ***p<0.0005. One-way ANOVA with a Tukey's posttest.

FIG. 6A-6L. Functional differences between T cells from TRex mice and P14 mice. FIG. 6A) Representative plots gated on live CD45+ splenic mononuclear cells from 1045^{+/+} MsIn^{-/-} and 7431^{+/+} MsIn^{-/-} mice. Quantified data are below. Data are mean±S.E.M. n=4-6 mice per group. *p<0.05, **p<0.005, and ***p<0.0005. One-way ANOVA with a Tukey's posttest. FIG. 6B) Representative Vβ9 (1045 and 7431) and Va2 (P14) staining on CD8 T cells and quantification. FIG. 6C) Phenotype of TCR KI splenic CD8+ T cells. FIG. 6D) Representative Vβ9 (1045 and 7431) and Va2 (P14) staining on CD4 T cells and quantification. Data are mean±S.E.M. n=4-6 mice per group. *p<0.05, **p<0.005, and ***p<0.0005. One-way ANOVA with a Tukey's posttest. FIG. 6E) Representative Vβ9 and MsIn₄₀₃₋₄₁₄:H-2D^{b} tetramer staining gated on CD8 T cells. FIG. 6F) Mean fluorescence intensity (MFI) of the indicated markers on day 6. Data are mean±S.E.M. n=4-6 mice per group. *p<0.05, **p<0.005, and ***p<0.0005. One-way ANOVA with a Tukey's posttest. FIG. 6G) Frequency of CD8 T cells co-producing IFNγ and TNFα and maximal response following incubation of effector T cells with APCs pulsed with titrating concentrations of specific peptides. Data are 3 independent animals pooled and are mean±S.E.M. FIG. 6H) MFI of the indicated cytokines of effector CD8 T cells incubated with APCs pulsed with titrating concentrations of specific peptides. FIG. 6I) Quantified data from CD8 and TCR downregulation following a 5 h incubation with antigen. FIG. 6J) Representative Vβ9 and MsIn₄₀₆₋₄₁₄:H-2D^{b} tetramer staining gated on CD4 T cells. MFI is quantified. Data are mean±S.E.M. n=4-6 mice per group. *p<0.05, **p<0.005, and ***p<0.0005. One-way ANOVA with a Tukey's posttest. FIG. 6K) Frequency of CD4 T cells co-producing IFNγ and TNFα and maximal response following incubation of effector T cells with APCs pulsed with titrating concentrations of specific peptides. Data are 3 independent animals pooled and are mean±S.E.M. FIG. 6L) MFI of the IFNγ of CD4 T cells incubated with APCs pulsed with titrating concentrations of specific peptides.

FIG. 7A-7E. Bias toward Tregs in MHC class I TCR transgenic mice but not TRex mice. FIG. 7A) Quantified data from FACS plots gated on live CD4+ splenic T cells. Data are mean±S.E.M. n=4-7 mice per group. *p<0.05, **p<0.005, and ***p<0.0005. One-way ANOVA with a Tukey's posttest. FIG. 7B) Quantified data from plots gated on live CD4+ Foxp3+ splenic T cells (Treg). Data are mean±S.E.M. n=4-7 mice per group. *p<0.05, **p<0.005, and ***p<0.0005. One-way ANOVA with a Tukey's posttest. FIG. 7C) Frequency of Foxp3+ Treg that express the engineered TCR. Quantified data is gated on Foxp3+ Treg. Data are mean±S.E.M. n=4-7 mice per group. *p<0.05, **p<0.005, and ***p<0.0005. One-way ANOVA with a Tukey's posttest. FIG. 7D) Frequency of Foxp3+ Treg of CD4 T cells in WT and OT1 mice. FIG. 7E) Frequency of splenic CD25-Foxp3+ Treg of total Treg in WT and OT1 mice. Graphed data are mean±S.E.M. n=3 mice per group. Statistical analysis was performed by an ANOVA with a Tukey's posttest to correct for multiple comparisons. *, p<0.05; **, p<0.005.

FIGS. 8A-B. Sequences of the TCR for the 1045 (FIG. 8A) (SEQ ID NO: 1) and the 7431 (FIG. 8B) (SEQ ID NO: 2) clones.

FIGS. 9A-9R. T cell development in P14 TRex mice faithfully is similar to wild type T cells. FIG. 9A) Frequency of EL4 cells that express Vβ8 and CD3 on day 3 post electroporation with Trac gRNA 2+Cas9 RNP with or without rAAV-P14. No zap, negative control. FIG. 9B) Donor P14 TCR integration into Trac was determined by a junction PCR.EL4 DNA (left image) or representative P14 TRex pups (right image). KI, TCR Trac knock-in. Pink arrow indicates P14 heterozygous red arrow indicates P14 homozygous (P14+/+) TRex pups. WT, wild type at both Trac alleles. FIG. 9C) Summary of overall frequency of TRex pups with the indicated genotype. FIG. 9D) Frequency of circulating CD4 and CD8 T cells (top, gated on live CD45+ cells) and frequency of Va2+Vp8+ among CD8 T cells from P14 TRex pups. FIG. 9E) Thymus weight in grams (g) and CD45 cell number per thymus from WT, P14 transgenic (Tg) or P14+/+ TRex mice. FIG. 9F) Representative plots gated of CD45+B220− thymocytes. FIG. 9G) Frequency (top) and number (bottom, per thymus) of double negative (DN), double positive (DP), CD4 single positive (CD4 SP) and CD8 single positive (CD8 SP) thymocytes. Data are mean±S.E.M. n=4 mice per group. *p<0.05, **p<0.005, ***p<0.0005, ****p<0.0001. FIG. 9H) Mean frequency of each subset among total CD45+B220−. FIG. 9I) Mean frequency of DN1-DN4 subsets among total DN. FIG. 9J) Representative DN1-DN4 plots are gated on CD4− CD8− DN thymocytes. CD44+ CD25− (DN1), CD44+ CD25+(DN2), CD44− CD25+(DN3), and CD44− CD25− (DN4). FIG. 9K) Representative plots of Vβ8+Vα2+ staining gated on the indicated thymocyte subset. FIG. 9L) Proportion of the indicated subsets that are Vβ8+. Data are mean±S.E.M. n=4 mice per group. **p<0.005, ****p<0.0001. FIG. 9M) Proportion of the indicated DN subsets that are Vβ8+. Data are mean±S.E.M. n=4 mice per group. *p<0.05, **p<0.005, ****p<0.0001. FIG. 9N. Representative plots of the indicated thymocyte subsets that express exogenous (Vβ8+) and/or endogenous (panVβ+) TCRβ. FIG. 9O) Frequency (top row) and number (bottom row, per thymus) of CD3+ CD8 SP that express exogenous (Vβ8+) and/or endogenous (panVβ+). Data are mean±S.E.M. n=4 mice per group. *p<0.05, ****p<0.0001. FIG. 9P) Mean proportion of CD3+ CD8 SP that express exogenous (Vβ8+) and/or endogenous (panVβ+) TCRβ in thymus or in blood. FIG. 9Q) Frequency of mature CD69− CD8 SP thymocytes among total CD3+ CD8 SP and single or dual TCRβ+ CD3+ CD8SP. Data are mean±S.E.M. n=4 mice per group. *p<0.05, **p<0.005, ***p<0.0005, ****p<0.0001. FIG. 9R) Number of CD69− CD3+ CD8 SP per thymus (left) and number of single or dual TCRβ+ CD69− CD3+ CD8 SP thymocytes. Data are mean±S.E.M. n=4 mice per group. *p<0.05, ****p<0.0001. Significance was determined using a one-way Anova with a Tukey's posttest.

FIGS. 10A-10H. Targeting a TCR to the Trac locus increases exogenous TCR expression and antigen sensitivity. FIG. 10A) Frequency (top row) and number (bottom row) of CD4 and CD8 T cells in spleen. Each dot is an independent mouse. Data are mean±S.E.M. n=4 mice per group. *p<0.05, **p<0.005, ****p<0.0001. ***p<0.0005. One-way ANOVA with a Tukey's posttest. FIG. 10B) Representative plots gated on splenic CD8 (top row) or CD4 (bottom row) T cells. FIG. 10C) Frequency (top row) or number (bottom row) of Vα2+Vβ8+ T cells. Each dot is an independent mouse. Data are mean±S.E.M. n=4 mice per group. **p<0.005, ****p<0.0001. ***p<0.0005. One-way ANOVA with a Tukey's posttest. FIG. 10D) Histograms of the indicated markers ex vivo (day 0) and day 6 post in vitro activation with gp33 peptide and IL-2. Representative of n=3-4 mice per group. FIG. 10E) Histograms of TCR chains and CD3 at the indicated hours post activation with 2 μg/ml of gp33 peptide. Data are quantified (right) and are mean±S.E.M. n=4 mice per group. Significance was determined using a Multiple Unpaired T-test with Welch correction a False Discovery Rate (FDR) of 1% and a Two-stage step-up (Benjamini, Krieger, and Yekutieli). **p<0.005. ***p<0.0005, ****p<0.0001. FIG. 10F) Proliferation gated on CD8 T cells on day 3 post activation with titrating doses of gp33 peptide (y-axis) and rhIL-2 (10 ng/ul). Significance was determined using a multiple unpaired T-test with Welch correction a False discovery rate (FDR) of 1% and a two-stage step-up (Benjamini, Krieger, and Yekutieli). **p<0.005. ***p<0.0005, ****p<0.0001. FIG. 10G) Proliferation gated on CD8 T cells on day 3 post activation with titrating doses of gp33 peptide (y-axis) without exogenous IL-2. FIG. 10H). Proportion of CD8+Vβ8+ T cells among each division cycle on day 3 post activation. Data are mean. n=4 mice per group. FIG. 10I) Proportion CD8 T cells producing IFNγ and expressing CD25 (plots) or CD69 (graphed data on right) on day post activation. Data are mean±S.E.M. n=4 mice per group. Significance was determined using a multiple Unpaired T-test with Welch correction a false discovery rate (FDR) of 1% and a two-stage step-up (Benjamini, Krieger, and Yekutieli). **p<0.005. ***p<0.0005, ****p<0.0001.

### DETAILED DESCRIPTION

To address issues with random DNA integration and lack of physiological regulation in methods of engineering T cell receptors for research and therapeutic purposes, a highly efficient method to directly replace endogenous TCRs with an engineered TCR was developed. To directly compare T cell development and functionality in ‘targeted’ vs. ‘random’ TCR integrated T cells with the same antigen specific TCR, we generate TRex mice (e.g., P14) to compare to historical TCR transgenic mice. Our results support that the TRex approach has advantages over traditional TCR transgenics and describe novel tools for study of physiological and antigen-specific T cells in diverse biological contexts.

The improved method replaces endogenous TCRs while disrupting endogenous genes (e.g., MsIn) concurrently using recombinant viral vector (e.g., rAAV) and a nuclease editing system. Two novel mouse strains were created in which a high or low affinity murine MsIn-specific TCR replaced endogenous TCRs within the Trac locus. These TCR-exchanged (TRex) mice provide several advantages over traditional TCR transgenic mice, and provide a physiologic and standardized source of MsIn-specific T cells to address the therapeutic challenges for targeting carcinomas.

### Definitions

Unless otherwise defined, all technical and scientific terms used herein have the same meaning as commonly understood by one of ordinary skill in the art to which this invention belongs. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991).

Each publication, patent application, patent, and other references cited herein is incorporated by reference in its entirety to the extent that it is not inconsistent with the present disclosure.

It is noted here that as used in this specification and the appended claims, the singular forms “a,” “an,” and “the” include plural reference unless the context clearly dictates otherwise.

“Amplification” refers to any means by which a polynucleotide sequence is copied and thus expanded into a larger number of polynucleotide molecules, e.g., by reverse transcription, polymerase chain reaction, and ligase chain reaction.

“cDNA” refers to a DNA that is complementary or identical to an mRNA, in either single stranded or double stranded form.

Conventional notation is used herein to describe polynucleotide sequences: the left-hand end of a single-stranded polynucleotide sequence is the 5-end; the left-hand direction of a double-stranded polynucleotide sequence is referred to as the 5-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5-end of the RNA transcript are referred to as “upstream sequences”; sequences on the DNA strand having the same sequence as the RNA and which are 3′ to the 3′ end of the coding RNA transcript are referred to as “downstream sequences.”

“Complementary” refers to the topological compatibility or matching together of interacting surfaces of two polynucleotides. Thus, the two molecules can be described as complementary, and furthermore, the contact surface characteristics are complementary to each other. A first polynucleotide is complementary to a second polynucleotide if the nucleotide sequence of the first polynucleotide is identical to the nucleotide sequence of the polynucleotide binding partner of the second polynucleotide. Thus, the polynucleotide whose sequence 5-TATAC-3′ is complementary to a polynucleotide whose sequence is 5′-GTATA-3′. A nucleotide sequence is “substantially complementary” to a reference nucleotide sequence if the sequence complementary to the subject nucleotide sequence is substantially identical to the reference nucleotide sequence.

“Conservative substitution” refers to the substitution in a polypeptide of an amino acid with a functionally similar amino acid. The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (WN).

The term “fragment” when used in reference to polypeptides refers to polypeptides that are shorter than the full-length polypeptide by virtue of truncation at either the N-terminus or C-terminus of the protein or both, and/or by deletion of an internal portion or region of the protein. Fragments of a polypeptide can be generated by methods known in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Expression control sequence” refers to a nucleotide sequence in a polynucleotide that regulates the expression (transcription and/or translation) of a nucleotide sequence operatively linked thereto. “Operatively linked” refers to a functional relationship between two parts in which the activity of one part (e.g., the ability to regulate transcription) results in an action on the other part (e.g., transcription of the sequence). Expression control sequences can include, for example and without limitation, sequences of promoters (e.g., inducible or constitutive), enhancers, transcription terminators, a start codon (i.e., ATG), splicing signals for introns, and stop codons.

The term “promoter” as used herein refers to a region of DNA that functions to control the transcription of one or more DNA sequences, and that is structurally identified by the presence of a binding site for DNA-dependent RNA-polymerase and of other DNA sequences, which interact to regulate promoter function. A functional expression promoting fragment of a promoter is a shortened or truncated promoter sequence retaining the activity as a promoter. Promoter activity may be measured in any of the assays known in the art e.g., in a reporter assay using Luciferase as reporter gene, or commercially available.

The term “vector” refers to any carrier of exogenous DNA or RNA that is useful for transferring exogenous DNA to a host cell for replication and/or appropriate expression of the exogenous DNA by the host cell. “Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in vitro expression system. Expression vectors include all those known in the art, such as cosmids, plasmids (e.g., naked or contained in liposomes) and viruses that incorporate the recombinant polynucleotide.

“Expression cassette” or “cassette” refers to a component of vector DNA that controls expression of a gene or protein, and may be interchangeable and easily inserted or removed from a vector. Expression cassettes often comprises a promoter sequence, an open reading frame, and a 3′ untranslated region that contains a polyadenylation site.

An “enhancer region” refers to a region of DNA that functions to increase the transcription of one or more genes. More specifically, the term “enhancer”, as used herein, is a DNA regulatory element that enhances, augments, improves, or ameliorates expression of a gene irrespective of its location and orientation. It is contemplated that an enhancer may enhance expression of more than one promoter.

“Polynucleotide” refers to a polymer composed of nucleotide units. Polynucleotides include naturally occurring nucleic acids, such as deoxyribonucleic acid (“DNA”), including cDNA, and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. The term “protein” typically refers to large polypeptides. The term “peptide” typically refers to short polypeptides. Conventional notation is used herein to portray polypeptide sequences: the left-hand end of a polypeptide sequence is the amino-terminus; the right-hand end of a polypeptide sequence is the carboxyl-terminus.

“Recombinant polynucleotide” refers to a polynucleotide having sequences that are not naturally joined together. An amplified or assembled recombinant polynucleotide may be included in a suitable vector, and the vector can be used to transform a suitable host cell. A host cell that comprises the recombinant polynucleotide is referred to as a “recombinant host cell.” The gene is then expressed in the recombinant host cell to produce, e.g., a “recombinant polypeptide.” A recombinant polynucleotide may serve a non-coding function (e.g., promoter, origin of replication, ribosome-binding site, etc.) as well. Recombinant protein refers to a protein encoded by a recombinant polynucleotide.

“Substantially pure” or “isolated” means an object species is the predominant species present (i.e., on a molar basis, more abundant than any other individual macromolecular species in the composition), and a substantially purified fraction is a composition wherein the object species comprises at least about 50% (on a molar basis) of all macromolecular species present. Generally, a substantially pure composition means that about 80% to 90% or more of the macromolecular species present in the composition is the purified species of interest. The object species is purified to essential homogeneity (contaminant species cannot be detected in the composition by conventional detection methods) if the composition consists essentially of a single macromolecular species. Solvent species, small molecules (<500 Daltons), stabilizers (e.g., BSA), and elemental ion species are not considered macromolecular species for purposes of this definition. In some embodiments, the lysosomal sulfatase enzymes of the invention are substantially pure or isolated. In some embodiments, the lysosomal sulfatase enzymes of the invention are substantially pure or isolated with respect to the macromolecular starting materials used in their synthesis. In some embodiments, the pharmaceutical composition of the invention comprises a substantially purified or isolated therapeutic lysosomal sulfatase enzyme admixed with one or more pharmaceutically acceptable carriers, diluents or excipients.

The term “specifically binds” is “antigen specific”, is “specific for”, “selective binding agent”, “specific binding agent”, “antigen target” or is “immunoreactive” with an antigen refers to a T cell receptor or polypeptide that binds a target antigen with greater affinity than other antigens of related proteins.

The term “T cell receptor” or “TCR” as used herein refers to a multisubunit protein comprising either α and β chains (TCR αβ) which together bind to a peptide-MHC ligand, or γ and δ subunits (TCRγδ). Each chain is composed of two extracellular domains comprising variable (V) region and a constant (C) region. The variable region binds to the peptide/MHC complex. The variable domain of both the TCR α-chain and β-chain each have three hypervariable or complementarity-determining regions (CDRs). The TCRαβ is complexed with CD3 and other proteins in the T cell to mediate signaling through the T cell receptor. High-affinity TCRs (Affinity ≥2.5 nM) are specific and sensitive for targeting cell-surface human LA.

The term “endogenous” refers to a protein, polynucleotide, or other molecule that is naturally found in or expressed by a subject, e.g., a cell, organ, or tissue. The term “exogenous” refers to a protein, polynucleotide, or other molecule that is not naturally found in a subject, e.g., a cell, organ, or tissue.

The term “genetically engineered” as used herein refers to a polynucleotide or polypeptide sequence that has been modified from its naturally-occurring sequence, e.g., by insertion, deletion or polynucleotide or amino acid substitution/modification, using recombinant DNA expression techniques to produce a polypeptide or polynucleotide sequence that differs from the previously unmodified sequence.

The term “nuclease dependent cleavage system” as used herein refers to gene editing techniques that employ DNA or RNA dependent nucleases to cleave target DNA or RNA, respectively, and molecules or guides that direct the nuclease to the target DNA/RNA to be cleaved. Examples of nuclease dependent cleavage systems include CRISPR/Cas systems, Cas-CLOVER systems, zinc-finger nuclease (ZFN) systems, transcription activator like effector nuclease (TALEN) systems, or meganuclease systems.

“Homozygous” for the donor TCR as used herein refers to the result of the genetic modification in which both alleles of the TCR express the donor TCR polynucleotide. “Heterozygous” for the donor TCR as used herein refers to the result of the genetic modification in which only one of the alleles of the TCR express the donor TCR polynucleotide.

### Nuclease Dependent Cleavage Systems

Zinc-finger nucleases (ZFNs) and Transcription activator-like effector nucleases (TALENs) are customizable DNA-binding proteins that comprise DNA-modifying enzymes. Both can be designed and targeted to specific sequences in a variety of organisms (Esvelt and Wang, Mol Syst Biol. (2013) 9: 641). ZFNs and TALENs are useful to introduce a broad range of genetic modifications by inducing DNA double-strand breaks that stimulate error-prone non-homologous end joining (NHEJ) or homology-directed repair (HDR) at specific genomic locations. These DNA-binding modules can be combined with numerous effector domains to affect genomic structure and function, including nucleases, transcriptional activators and repressors, recombinases, transposases, DNA and histone methyltransferases, and histone acetyltransferases. Thus, the ability to execute genetic alterations depends largely on the DNA-binding specificity and affinity of designed zinc finger and TALEN proteins (Gaj et al., Trends in Biotechnology, (2013) 31(7):397-405). The following U.S. granted patents, incorporated by reference, describe the use of ZFNs and TALENs in mammalian cells, U.S. Pat. Nos. 8,685,737 and 8,697,853.

CRISPR-Cas (clustered regularly interspaced short palindromic repeats-CRISPR associated) is an RNA-mediated adaptive immune system found in bacteria and archaea, which provides adaptive immunity against foreign nucleic acids (Wiedenheft et al., Nature (2012) 482:331-8; Jinek et al., Science (2012) 337:816-21). Recent studies have shown that the biological components of this system can be used to modify to the genome of mammalian cells. CRISPR-Cas systems are generally defined by a genomic locus called the CRISPR array, a series of 20-50 base-pair (bp) direct repeats separated by unique “spacers” of similar length and preceded by an AT-rich “leader” sequence (Wright et al., Cell (2016) 164:29-44).

Three types of CRISPR/Cas systems exist, type 1, ∥ and Ill. The Type II CRISPR-Cas systems require a single protein, Cas9, to catalyze DNA cleavage (Sapranauskas et al., Nucleic Acids Res. (2011) 39(21): 9275-9282). Cas9 serves as an RNA-guided DNA endonuclease. Cas9 generates blunt double-strand breaks (DSBs) at sites defined by a 20-nucleotide guide sequence contained within an associated CRISPR RNA (crRNA) transcript. Cas9 requires both the guide crRNA and a trans-activating crRNA (tracrRNA) that is partially complementary to the crRNA for site-specific DNA recognition and cleavage (Deltcheva et al., Nature (2011)4 71(7340):602-7; Jinek et al., Science (2012) 337:816-21).

The crRNA:tracrRNA complex can be synthesized as two separate molecules or as a single transcript (single-guide RNA or sgRNA) encompassing the features required for both Cas9 binding and DNA target site recognition. Using sgRNA, Cas from bacterial species, such as S. pyogenes, can be programmed to cleave double-stranded DNA at any site defined by the guide RNA sequence and including a protospacer-adjacent (PAM) motif (Sapranauskas et al., Nucleic Acids Res. (2011) 39(21): 9275-9282; Jinek et al., Science (2012) 337:816-21). The DSBs result in either non-homologous end-joining (NHEJ), which is error-prone and conducive to frameshift mutations that knock out gene alleles, or homology-directed repair (HDR), which can be exploited with the use of an exogenously introduced double-strand or single-strand DNA repair template to knock in or correct a mutation in the genome. Therefore, in the presence of a homologous repair donor, the CRISPR/Cas9 system may be used to generate precise and defined modifications and insertions at a targeted locus through the HDR process. In the absence of a homologous repair donor, single DSBs generated by CRISPR/Cas9 are repaired through the error-prone NHEJ, which results in insertion or deletion (indel) mutations.

Other publications describing the CRISPR systems and Cas9, include the following: Cong et al. Science (2013) 339:819-23; Jinek et al., eLife 2013; 2:e00471. (2013) 2:e00471; Lei et al. Cell (2013) 152: 1173-1183; Gilbert et al. Cell (2013) 154:442-51; Lei et al. eLife (2014) 3:e04766; Perez-Pinela et al. Nat Methods (2013) 10: 973-976; Maider et al. Nature Methods (2013) 10, 977-979 which are incorporated by reference. The following U.S. and international patents and patent applications describe the methods of use of CRISPR, U.S. Pat. Nos. 8,697,359; 8,771,945; 8,795,965; 8,865,406; 8,871,445; 8,889,356; 8,895,308; 8,906,616; 8,932,814; 8,945,839; 8,993,233; 8,999,641; 2014/0068797; and WO 2014/197568, each of which is incorporated by reference in their entirety.

The CRISPR related protein, Cas9, can be from any number of species including, but not limited to, Streptococcus pyogenes, Staphylococcus aureus, Listeria innocua, and Streptococcus thermophilus.

Additional Cas proteins known in the art are contemplated for use in the methods, including Cas12a (Cpf1) and Cas 13a/Cas13b (56). See also Yan et al., Cell Biology and Toxicology 35:489-492 (2019).

Cas-CLOVER™ systems are recently designed gene editing systems that utilize the Clo51 nuclease instead of the CRISPR protein. Cas-CLOVER™ comprises a nuclease-inactivated Cas9 protein fused to the Clo51 endonuclease (55). Cas-CLOVER uses two guide RNAs as well as a nuclease activity that requires dimerization of subunits associated with each guide RNA to provide target specificity.

In one embodiment, the methods use a CRISPR-Cas system and one or more guide RNAs, repair templates and HDR to insert nucleotide bases into the genome of a TCR locus.

### Nucleic Acid Molecules

Nucleic acids of the disclosure can be cloned into a vector, such as a plasmid, cosmid, bacmid, phage, artificial chromosome (BAC, YAC) or virus, into which another genetic sequence or element (either DNA or RNA) may be inserted so as to bring about the replication of the attached sequence or element. In some embodiments, the expression vector contains a constitutively active promoter segment (such as but not limited to CMV, SV40, Elongation Factor or LTR sequences) or an inducible promoter sequence such as the steroid inducible pIND vector (Invitrogen), where the expression of the nucleic acid can be regulated. Expression vectors of the invention may further comprise regulatory sequences, for example, an internal ribosomal entry site. The vector can be introduced into a cell or embryo by transfection, for example.

A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. For instance, in some embodiments, signal peptide sequences may be appended/fused to the amino terminus of any of the TCR, CRISPR-Cas or other nuclease-dependent cleavage system described herein.

### Vectors

A wide range of host-vector systems suitable for the expression of engineered TCR or fragments thereof are available.

In various embodiments, the vectors are adenovirus vectors, adeno-associated virus vectors or retroviral vectors.

In various embodiments, the vectors are adenovirus vectors. “Adenovirus expression vector” is meant to include constructs containing adenovirus sequences sufficient to (a) support packaging of the construct in host cells with complementary packaging functions and (b) to ultimately express a heterologous gene of interest that has been cloned therein. The adenovirus may be of any of the 42 different known serotypes or subgroups A-F.

Adenoviral infection of host cells does not result in chromosomal integration because wild-type adenoviral DNA can replicate in an episomal manner without potential genotoxicity. Also, adenoviruses are structurally stable, and no genome rearrangement has been detected after extensive amplification. Adenovirus is useful as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation.

In various embodiments, the methods contemplate delivery of selected genes to target sites through the use of adeno associated virus (AAV) vectors. AAV comprises a single-stranded DNA genome, but lacks the essential genes needed for replication and expression on its own. These functions are provided by the Ad E1, E2a, E4, and VA RNA genes. There are 12 known serotypes of AAV in primates categorized into five main clades (Clades A-E). Examples of adeno-associated virus vectors useful in the methods include AAV1, AAV2, AAV3, AAV4, AAV5, AAV6, AAV9 and AAV-DJ.

In various embodiments, the methods contemplate delivery of selected genes to target sites through the use of retrovirus vectors. Retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. Examples of retroviruses useful in the methods include lentiviruses.

### Cell Culture Methods

Mammalian cells containing the recombinant protein-encoding DNA or RNA are cultured under conditions appropriate for growth of the cells and expression of the DNA or RNA. Those cells which express the recombinant protein can be identified, using known methods and methods described herein, and the recombinant protein can be isolated and purified, using known methods and methods also described herein, either with or without amplification of recombinant protein production. Identification can be carried out, for example, through screening genetically modified mammalian cells that display a phenotype indicative of the presence of DNA or RNA encoding the recombinant protein, such as PCR screening, screening by Southern blot analysis, or screening for the expression of the recombinant protein. Selection of cells which contain incorporated recombinant protein-encoding DNA may be accomplished by including a selectable marker in the DNA construct, with subsequent culturing of transfected or infected cells containing a selectable marker gene, under conditions appropriate for survival of only those cells that express the selectable marker gene. Further amplification of the introduced DNA construct can be effected by culturing genetically modified mammalian cells under appropriate conditions (e.g., culturing genetically modified mammalian cells containing an amplifiable marker gene in the presence of a concentration of a drug at which only cells containing multiple copies of the amplifiable marker gene can survive).

Genetically modified mammalian cells expressing the recombinant protein can be identified, as described herein, by detection of the expression product.

Protein purification methods are known in the art and utilized herein for recovery of recombinant proteins from cell culture media. For example, methods of protein and antibody purification are known in the art and can be employed with production of the antibodies of the present disclosure. In some embodiments, methods for protein and antibody purification include filtration, affinity column chromatography, cation exchange chromatography, anion exchange chromatography, and concentration. The filtration step may comprise ultrafiltration, and optionally ultrafiltration and diafiltration. Filtration is preferably performed at least about 5-50 times, more preferably 10 to 30 times, and most preferably 14 to 27 times. Affinity column chromatography, may be performed using, for example, PROSEP® Affinity Chromatography (Millipore, Billerica, Mass.). In various embodiments, the affinity chromatography step comprises PROSEP®-vA column chromatography. Eluate may be washed in a solvent detergent. Cation exchange chromatography may include, for example, SP-Sepharose Cation Exchange Chromatography. Anion exchange chromatography may include, for example but not limited to, Q-Sepharose Fast Flow Anion Exchange. The anion exchange step is preferably non-binding, thereby allowing removal of contaminants including DNA and BSA. The antibody product is preferably nanofiltered, for example, using a Pall DV 20 Nanofilter. The antibody product may be concentrated, for example, using ultrafiltration and diafiltration. The method may further comprise a step of size exclusion chromatography to remove aggregates.

### Host Cells

Suitable host cells for the expression of engineered TCR are derived from multicellular organisms. Examples of useful mammalian host cell lines are Chinese hamster ovary cells, including CHOK1 cells (ATCC CCL61), DXB-11, DG-44, and Chinese hamster ovary cells/-DHFR (CHO, Urlaub et al., PNAS 77:4216 (1980)); monkey kidney CV1 line transformed by SV40 (COS-7, ATCC CRL 1651); human embryonic kidney line (293 or 293 cells subcloned for growth in suspension culture, (Graham et al., J. Gen Virol. 36:59, 1977); baby hamster kidney cells (BHK, ATCC CCL 10); mouse sertoli cells (TM4, Mather, (Biol. Reprod. 23:243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y Acad. Sci. 383:44-68 (1982)); MRC 5 cells; FS4 cells; and a human hepatoma line (Hep G2).

Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present disclosure, particularly for transfection of Spodoptera frugiperda cells.

Host cells are transformed or transfected with the above-described expression or cloning vectors for antibody production and cultured in conventional nutrient media modified as appropriate for inducing promoters, selecting transformants, or amplifying the genes encoding the desired sequences. In addition, vectors and transfected cell lines with multiple copies of transcription units separated by a selective marker are particularly useful and preferred for the expression of antibodies that bind target.

### Methods of Use

The engineered TCR of the present disclosure are useful to study the immunological effects of T cells expressing an antigen in the context of the T cell receptor and the ability of the antigen to stimulate downstream immunological responses. The engineered TCR herein provide information on immunological responses to antigen and are useful to develop therapeutics toward the antigens.

In various embodiments, the engineered TCR comprises an antigen that is a cancer antigen, a tumor specific antigen, a neo antigen, an autoimmune antigen, a microbial antigen, a viral antigen, a bacterial antigen.

In various embodiments, the cancer is a solid tumor or a blood cancer. In various embodiments, the cancer is selected from the group consisting of leukemias, brain tumors (including meningiomas, glioblastoma multiforme, anaplastic astrocytomas, cerebellar astrocytomas, other high-grade or low-grade astrocytomas, brain stem gliomas, oligodendrogliomas, mixed gliomas, other gliomas, cerebral neuroblastomas, craniopharyngiomas, diencephalic gliomas, germinomas, medulloblastomas, ependymomas. choroid plexus tumors, pineal parenchymal tumors, gangliogliomas, neuroepithelial tumors, neuronal or mixed neuronal glial tumors), lung tumors (including small cell carcinomas, epidermoid carcinomas, adenocarcinomas, large cell carcinomas, carcinoid tumors, bronchial gland tumors, mesotheliomas, sarcomas or mixed tumors), prostate cancers (including adenocarcinomas, squamous cell carcinoma, transitional cell carcinoma, carcinoma of the prostatic utricle, or carcinosarcomas), breast cancers (including adenocarcinomas or carcinoid tumors), or gastric, intestinal, or colon cancers (including adenocarcinomas, invasive ductal carcinoma, infiltrating or invasive lobular carcinoma, medullary carcinoma, ductal carcinoma in situ, lobular carcinoma in situ, colloid carcinoma or Paget's disease of the nipple), skin cancer (including melanoma, squamous cell carcinoma, tumor progression of human skin keratinocytes, basal cell carcinoma, hemangiopericytoma and Karposi's sarcoma), lymphoma (including Hogkin's disease and non-Hodgkin's lymphoma), and sarcomas (including osteosarcoma, chondrosarcoma and fibrosarcoma).

In various embodiments, the cancer antigen is mesothelin, BCMA, CD19, CD20, CD22, CD70, CD123, CEA, CDH3, CLDN6, CLL1, CS1, DCAF4L2, FLT3, GABRP, MageB2, MART-1, MSLN, MUC1 (e.g., MUC1-C), MUC12, MUC13, MUC16, mutFGFR3, PRSS21, PSMA, RNF43, STEAP1, STEAP2, TM4SF5, PD-1, CTLA4, EGFR, VEGF, OX40, or FcRL5.

In various embodiments, the autoimmune disease is selected from the group consisting of achalasia, Addison's disease, adult still's disease, agammaglobulinemia, alopecia areata, amyloidosis, ankylosing spondylitis, anti-gbm/anti-tbm nephritis, antiphospholipid syndrome autoimmune angioedema autoimmune dysautonomia autoimmune encephalitis autoimmune hepatitis, autoimmune inner ear disease (AIED), autoimmune myocarditis autoimmune oophoritis, autoimmune orchitis, autoimmune pancreatitis, autoimmune retinopathy autoimmune urticarial, axonal & neuronal neuropathy (AMAN), Baló disease, Behcet's disease, benign mucosal pemphigoid (Mucous membrane pemphigoid), bullous pemphigoid, Castleman disease (CD), Celiac disease, Chagas disease, chronic inflammatory demyelinating polyneuropathy (CIDP), chronic recurrent multifocal osteomyelitis (CRMO), Churg-Strauss syndrome (CSS) or eosinophilic granulomatosis (EGPA), cicatricial pemphigoid, Cogan's syndrome, cold agglutinin disease, complex regional pain syndrome (formerly known as reflex sympathetic dystrophy), congenital heart block, coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (HS) (acne inversa), IgA nephropathy, IgG4-related sclerosing disease, immune thrombocytopenic purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, lichen planus, lichen sclerosus, ligneous conjunctivitis, linear IgA disease (LAD), lupus, Lyme disease chronic, Meniere's disease, microscopic polyangiitis (MPA), mixed connective tissue disease (MCTD), Mucha-Habermann disease, multifocal motor neuropathy (MMN) or MMNCB, multiple sclerosis, myasthenia gravis, myelin oligodendrocyte glycoprotein antibody disorder, myositis, narcolepsy, neonatal lupus, neuromyelitis optica/devic disease, neutropenia, ocular cicatricial pemphigoid, optic neuritis, palindromic rheumatism (PR), PANDAS (Pediatric autoimmune neuropsychiatric disorders associated with streptococcus infections), paraneoplastic cerebellar degeneration (PCD), paroxysmal nocturnal hemoglobinuria (PNH), pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes type I, II, III, polymyalgia rheumatic, polymyositis, postmyocardial infarction syndrome, postpericardiotomy syndrome, primary biliary cholangitis, primary sclerosing cholangitis, progesterone dermatitis, progressive hemifacial atrophy (PHA), Parry romberg syndrome, psoriasis, psoriatic arthritis, pure red cell aplasia (PRCA), pyoderma gangrenosum, Raynaud's phenomenon, reactive arthritis, relapsing polychondritis, restless legs syndrome (RLS), retroperitoneal fibrosis, rheumatic fever, rheumatoid arthritis, sarcoidosis, Schmidt syndrome or autoimmune polyendocrine syndrome type II, scleritis, scleroderma, Sjögren's Disease, stiff person syndrome (SPS), Susac's syndrome, sympathetic ophthalmia (SO), Takayasu's arteritis, temporal arteritis/giant cell arteritis, thrombocytopenic purpura (TTP), thrombotic thrombocytopenic purpura (Ttp), thyroid eye disease (Ted), Tolosa-Hunt syndrome (THS), transverse myelitis, type 1 diabetes, ulcerative colitis (UC), undifferentiated connective tissue disease (UCTD), uveitis, vasculitis, vitiligo, Vogt-Koyanagi-Harada disease, and warm autoimmune hemolytic anemia.

In various embodiments, the autoimmune antigen is associated with an autoimmune disease described herein.

Provided are methods of making a cell, e.g., a T cell, expressing a genetically engineered TCR comprising a T cell receptor exchanged (Trex) locus, or methods of making a genetically engineered non-human animal comprising or expressing via a germline insertion or a somatic insertion of an engineered TCR comprising a T cell receptor exchanged (Trex) locus.

In various embodiments, the disclosure contemplates a method of making an engineered T cell receptor comprising a T cell receptor exchanged (Trex) locus, the method comprising: i) expressing a T cell receptor gene comprising a T cell receptor alpha (Trac) locus on a plasmid or vector containing homologous sequence to the murine Trac locus; ii) inserting a donor TCR polynucleotide sequence into the T cell receptor alpha (Trac) locus of the T cell receptor gene using a nuclease-dependent cleavage system comprising Trac-specific targeting molecules specific to Trac exon 1 or directly 5′ to Trac exon 1 complexed to a ribonucleoprotein (RNP); and, iii) expressing the engineered TCR from the plasmid or vector.

Also provided herein is a method of making an engineered T cell receptor comprising a T cell receptor exchanged (Trex) locus, the method comprising i) expressing a T cell receptor gene comprising a T cell receptor alpha (Trac) locus on a plasmid or vector containing homologous sequence to the murine Trac locus; ii) inserting a donor TCR polynucleotide sequence into the T cell receptor alpha (Trac) locus of the T cell receptor gene using a CRISPR/Cas gene editing system comprising Trac-specific guide RNAs (gRNAs) specific to Trac exon 1 or directly 5′ to Trac exon 1 complexed to Cas ribonucleoprotein (RNP); and, iii) expressing the engineered TCR from the plasmid or vector. In various embodiments, the expression is from an endogenous locus.

Contemplated herein is a method of making a genetically engineered non-human animal comprising a T cell receptor exchanged (Trex) locus, the method comprising i) expressing a donor T cell receptor polynucleotide specific for a target antigen on a plasmid or vector; and ii) inserting the donor TCR polynucleotide of i) into T cell receptor alpha (Trac) locus of a T cell receptor gene in a non-human animal zygote using a nuclease-dependent cleavage system comprising Trac-specific targeting molecules specific to Trac exon 1 or directly 5′ to Trac exon 1 complexed to a ribonucleoprotein (RNP).

Further provided is a method of making a genetically engineered non-human animal comprising a T cell receptor exchanged (Trex) locus, the method comprising i) expressing a donor T cell receptor polynucleotide specific for a target antigen on a plasmid or vector; and ii) inserting the donor TCR polynucleotide of i) into T cell receptor alpha (Trac) locus of a T cell receptor gene in a non-human animal zygote using a CRISPR/Cas gene editing system comprising Trac-specific guide RNAs (gRNAs) specific to Trac exon 1 (Trac gRNA 1) or directly 5′ to Trac exon 1 (Trac gRNA 2) complexed to a Cas ribonucleoprotein (RNP).

In various embodiments, the donor TCR sequences comprise a TCRβ variable (V), TCRβ Constant (C) and TCRα V sequence. In various embodiments, the exogenous TCRβ, TCRα, and endogenous Trac sequences are linked by self-cleaving 2A element.

In various embodiments, when a CRISPR/Cas system is used the guide RNAs are nucleofected into activated splenic polyclonal T cells.

In various embodiments, the donor TCR sequence is encoded in an AAV vector. In various embodiments, the donor TCR sequence is flanked by approximately 250 to 1000 bp homology arms (HA) encoding endogenous murine Trac sequences and cloned into an AAV vector. In various embodiments, the AAV is AAV6, AAV1 or AAV-DJ.

In various embodiments, the Cas protein is a Cas9, Cas12a, Cas13a or Cas13b. In various embodiments, the Cas is cas9 and CRISPR/Cas9 initiates a double-strand DNA break directly upstream of Trac or in exon 1.

In various embodiments, T cells expressing a TRex TCR specific for the target antigen upon activation upregulate CD44 and maintain CD62L levels, downregulate TCR, and/or minimally express PD-1.

In various embodiments, when making a genetically engineered non-human animal, rAAV expressing the TRex locus is administered to embryos at a final concentration of between 1.0×10⁸ GC/μl and 3×10⁸ GC/μl.

In various embodiments, the method further comprises inactivating a gene encoding the target antigen of interest in the non-human animal. In various embodiments, the gene encoding the target antigen is inactivated using a nuclease-dependent cleavage system.

In various embodiments, 80% or more of CD4 and/or CD8 T cells in the genetically engineered non-human animal express an engineered TCR.

In various embodiments, the T cells expressing the Trex TCR are not tolerized to the target antigen. In various embodiments, T cells expressing the Trex TCR upon activation upregulate CD44 and maintain CD62L levels, downregulate TCR, and/or minimally express PD-1.

Also provided is a cell or a genetically engineered non-human expressing a T cell receptor comprising a T cell receptor exchanged (Trex) locus specific for a target antigen.

In various embodiments, the cell is a T cell, optionally wherein the T cell is a CD4+ T cell or CD8+ T cell. In various embodiments, the T cell is an effector T cell or a memory T cell.

In various embodiments, the T cell is CD44^{low}/CD62L+, CD44highCD26L− or CD44highCD62L+.

Further contemplated is a method of measuring effects of T cells having TCR exchanged (TRex) T cell receptor locus expressing a T cell receptor specific for a target antigen of interest comprising contacting a T cell comprising a T cell receptor exchanged (Trex) locus with the target antigen presented in MHC and measuring the effects on the T cell. In various embodiments, the measured effects include stimulation of cytokine production, modulation of cell surface marker phenotype, change in activation phenotype, modulation of number of regulatory T cells induced, or cytotoxicity phenotype, replicating endogenous TCR gene regulation following antigen encounter, and eliminating endogenous TRAC expression.

### Kits

The polynucleotides, plasmid system or vectors described herein may be provided in a kit. The kits may include, in addition to the polynucleotide, plasmid system or vector, any reagent which may be employed in the use of the system. In one embodiment, the kit includes reagents necessary for transformation of the vectors into mammalian cells. The kit may include growth media or reagents required for making growth media, for example, DMEM for growth of mammalian cells. Components supplied in the kit may be provided in appropriate vials or containers (e.g., plastic or glass vials). The kit can include appropriate label directions for storage, and appropriate instructions for usage.

### EXAMPLES

### Example 1—Materials and Methods

Animals: University of Minnesota Institutional Animal Care and Use Committee approved all animal studies. C57BL/6J mice were purchased directly from Jackson Labs (stock #000664). Pseudopregnant CD-1 female mice were purchased from Charles River Laboratory (stock #CD-1 022). Generation of TCR knockin (KI) animals was performed in the Mouse Genetic Laboratory at the University of Minnesota. P14 TCR transgenic (2, 10) and OT1 mice (19) have been previously described.

Cloning: The Trac targeting TCR vectors was produced by first designing ˜1 kb homology arms (HA) flanking the CRISPR gRNA target site in exon 1 such that transgenic mesothelin-specific TCRs, high affinity (clone 1045) or low affinity (clone 7431) MsIn₄₀₆₋₄₁₄:H-2D^{b}-specific, or P14 TCR are inserted in-frame. A Furin (RRKR)-GSG (SEQ ID NO: 3)-T2A element (51) was incorporated at the 5′ end of the TCR insert site to facilitate co-translational separation from the residual peptide sequence of the endogenous Trac locus. The Trac HA-GSG-T2A sequence was synthesized as a gBlock Gene Fragments (IDT, Coralville, IA) with AttB sites and subcloned into pDONR221 using the Gateway BP Clonase II Enzyme Mix (ThermoFisher Scientific, Waltham, MA) to produce pENTR-mTRAC HA. TCR sequences were codon optimized and synthesized by Genscript and subsequently cloned into pENTR-mTRAC HA using Gibson Assembly (52). Following sequence verification, the pENTR-mTrac HA-TCR was cloned into pAAV-Dest-pA using the Gateway LR Clonase II Enzyme Mix (ThermoFisher Scientific, Waltham, MA). pAAV constructs were then sent to Vigene (1045 TCR) or SignaGen (7431 TCR and P14 TCR) Laboratories for commercial AAV production. High titer virus ranged from 1.92-3×10¹³ gene copies (GC) per mL and was stored at −80° C.

Reagents: DNA encoding the high affinity MsIn₄₀₆₋₄₁₄:H-2D^{b} 1045 TCR (2) was cloned into a recombinant adeno-associated viral vector (rAAV) and high-titer was produced by Vigene. DNA encoding the lower affinity MsIn₄₀₆₋₄₁₄:H-2D^{b} 7431TCR (2) was cloned into rAAV and high titer virus was provided by Vigene or Signagen. Virus concentration were of 3×10¹³ gene copies (GC) per mL and rAAV was administered to embryos at a final concentration of 1.5×10⁸ GC/μl. TrueCut Cas9 (ThermoFisher Scientific, A36498) and gRNAs (Synthego) were combined to form RNPS at a 1:1 molar ratio prior to nucleofection. Two sgRNAs specific to murine Trac exon 1 were initially tested, Guide 1: UCUUUUAACUGGUACACAGC (−54220544) (SEQ ID NO: 4) and Guide 2: UUCUGGGUUCUGGAUGUCUG (−54220521) (SEQ ID NO: 5).

While both guides efficiently knocked out endogenous TCRs, only Trac Guide 2 resulted in exogenous TCR integration in murine polyclonal T cells and was therefore used in all subsequent experiments. Two gRNAs specific to murine MsIn exon 4 were initially tested, MsIn Guide 1: GGAGGUAUCUGACCUGAGCA (−25753010) (SEQ ID NO: 6) and MsIn Guide 2 GGCCAAGAAAGAGGCCUGUG (+25753054) (SEQ ID NO: 7) and validated in 3T3 cells. MsIn guide 2 was selected for all subsequent experiments.

Celllines: EL4 cells are derived from a lymphoma induced in a C57BL/6N mouse by 9,10-dimethyl-1,2-benzanthracene and are commercially available (TIB-93, ATCC). NIH/3T3 fibroblast cell line that was isolated from a mouse NIH/Swiss embryo and are commercially available (CRL-1658, ATCC). Both cell lines were cultured according to ATCC specifications.

Generating Cas9 RNPs: Synthego sgRNAs were resuspended at 50 μM. 7 μl TrueCut Cas9 v2 (5 g/mL, ThermoFisher Scientific, A36498) was combined with 7 μl TRAC sgRNA #2 and 7 μl MSLN sgRNA #2 at a 1:1 molar ratio and mixed gently by pipetting similar to as described (16). Cas9 and gRNA complexes were incubated at room temperature for 10 minutes to generate ribonucleoprotein (RNP) complexes and stored on ice during transfer to the University of Minnesota Mouse Genetic Laboratory.

Superovulation and rAAV incubation: 24-28-day old female C57BL/6J mice were purchased directly from Jackson Labs (stock #000664). A total of 10 C57BL/6J mice were superovulated by i.p. injection of 5 IU/mouse of Pregnant Mare Serum Gonadotropin (PMSG, C1063, Sigma). After 47-48 hours later, 5 IU/mouse of human Chorionic Gonadotropin (hCG, HOR-250, PROSPEC Protein Specialists) was injected i.p. in PMSG-treated females.

Superovulating females were immediately crossed with C57BL/6J males at a 1:1 ratio to produce 1-cell zygotes. The next morning, zygotes were collected and washed using standard methods (53). Briefly, zygotes were collected from the ampulla of the plugged females, treated in hyaluronidase (H4272, Sigma) in a 35 mm TC-treated dish (#353001, Falcon) containing 3.5 ml of modified Human Tubal Fluid (mHTF) (54) for 2 minutes to remove cumulus cells around the zygotes. The zygotes were then washed 2× in mHTF and then zona pellucida was thinned by briefly treating the zygotes in the Acidic Tyrode's solution (T1788, Sigma). Zygotes were subsequently washed 4× in M2 media (MR-051-F, Millipore), and incubated in 50 μl of mHTF containing rAAV (1.5×10⁸ GC/μl) covered by mineral oil (M8410, Sigma) in a 60 mm tissue culture dish (Ref: 353004, Falcon) for 6 hours at a 37° C., 5% CO₂.

Electroporation of zygotes with CRISPR Cas9 RNPs and rAAV-expressing 1045 or 7431 TCRs: TrueCut Cas9 (ThermoFisher Scientific, A36498) and gRNAs were combined at a 1:1 molar ratio prior to electroporation. Cas9+gRNA complexes were incubated at room temperature for 10 minutes to generate ribonucleoprotein (RNP) complexes and stored on ice during transfer to the University of Minnesota Mouse Genetics Laboratory. Following 6 h incubation with rAAV, zygotes were washed 1× in Reduced Serum Medium (OPTI-MEM, #31985-062, Gibco). A total of 91 zygotes were next mixed with 10 μl of OPTI-MEM, 9 μl of mHTF (containing rAAV at 1.5×10⁸ GC/μl) and 2 μl of 10× preformed RNP complex (Cas9+gRNAs to Trac and MsIn) sgRNA/Cas9 protein) complex. The electroporation was performed in a 1 mm gap electroporation cuvette (Cat #5510, Molecular BioProducts) using BioRad Xcell instrument according to following parameters: square wave at 30V, 6 pulses with 3 ms duration and 100 ms interval. After the electroporation, zygotes were washed one-time in 1× OPTI-MEM and then transferred to the original mHTF drop for overnight culture. The next day, 27 zygotes remained as 1-cell embryos 3 zygotes were Cysed. A total of 61 zygotes developed into 2-cell embryos, which were then transferred into 2 pseudopregnant CD-i females (Charles River Laboratory). A total of 15 pups were born 19 days later. This procedure was repeated with a higher rAAV concentration (2.25×10⁸) and no pups were born. Results are as follows for 1045 and 7431 KI:

| 1045 Virus | Total | # 2-cell | # 1-cell | # Lysed | # Pseudopregnant | |
|---|---|---|---|---|---|---|
| GC/μl | zygotes | embryos | embryos | zygotes | CD-1 females | # Pups |
| 1.5 × 10⁸ GC/μl | 91 | 61 | 27 | 3 | 2 | 15 |
| 2.25 × 10⁸ GC/μl | 72 | 44 | 20 | 8 | 1 | 0 |

| 7431 Virus | Total | # 2-cell | # 1-cell | # Lysed | # Pseudopregnant | |
|---|---|---|---|---|---|---|
| GC/μl | zygotes | embryos | embryos | zygotes | CD-1 females | # Pups |
| 1.5 × 10⁸ GC/μl | 261 | 206 | 43 | 7 | 4 | 13 |

| P14 Virus | Total | # 2-cell | # 1-cell | # Lysed | # Pseudopregnant | |
|---|---|---|---|---|---|---|
| GC/μl | zygotes | embryos | embryos | zygotes | CD-1 females | # Pups |
| 1.5 × 10⁸ GC/μl | 471 | 235 | 226 | 10 | 8 | 52 |

Mouse PCR Genotyping: Toe or ear snips were digested using the REDExtract Kit (Sigma Aldrich). PCR was run using Q5 HiFi Master Mix (New England Biolabs) for Trac KO, MsIn KO, and Trac Junction PCR protocols using the following gene-specific PCR primers purchased from IDT: Trac KO forward, 5′-GCTAGATCCTAGGCTGTCATTTC-3′ (SEQ ID NO: 8), Trac KO reverse, 5′-CCAATGTCCTCTGTCATGTTCT-3′ (SEQ ID NO: 9), with an amplicon length of 579 bp; MsIn KO forward, 5′-AGGTGGGTTCAGTACCTTTG-3′ (SEQ ID NO: 10), and MsIn KO reverse, 5′-GATCAGCTCAGACTTGGGATAG-3′ (SEQ ID NO: 11), with an amplicon length of 698 bp. Amplification was run for 30 cycles at 95° C. for 30 seconds, 55° C. for 30 seconds, 74° C. for 1 min. To assess exogenous TCR integration into the Trac locus, a Trac junction PCR protocol was created using the following gene-specific PCR primers: Wild type (WT) forward, 5′-CTCTGGTGTGAGTGCTATTC-3′ (SEQ ID NO: 12), 1045 and 7431 knock-in (KI) forward, 5′-CCTGTTCTGGTACGTGAGATAC-3′ (SEQ ID NO: 13), P14 KI forward, 5′-GTAGCTATGAGGATAGCACCTTT-3′ (SEQ ID NO: 14), and a junction universal reverse primer, 5′-CAAGAGAAGACAGGAAGGTGAG-3′. The WT amplicon length is 1025 bp and the KI amplicon length is 750 bp and the P14 KI amplicon length is 742 bp. Amplification was run for 30 cycles of 95° C. for 30 seconds, 60° C. for 30 seconds, and 74° C. for 1 minute. Trac and MsIn KO PCR products were purified using a PCR Clean-Up Kit (Qiagen) and were subsequently submitted for Sanger sequencing through Eurofins genomics using both forward and reverse primers. All PCR was run on an Eppendorf Vapo Protect thermocycler. Sequence results were analyzed using Snapgene and with Interference with Crispr Edits (ICE) software (Synthego, Menlo Park, CA). Mutant sequences were directly compared to WT control sequence. Trac junction PCR product was run on a 1.5% agarose gel and imaged in a UV transilluminator with ethidium bromide.

Primary murine T cell activation: Spleens were dissociated through a 40 μm filter using the backside of a sterile syringe. RBCs were lysed by resuspension in 1 ml ACK lysis buffer for 2 minutes. Lysis was quenched by addition of 10 mls of T cell media. T cells were centrifuged at 350×g for 5 minutes at 4° C. and resuspended in 10 ml of T cell media containing 10 ng/μl recombinant human IL-2 (rhIL-2, Peprotech), 5 ng/μl recombinant murine IL-7 (rmIL-7, R&D Systems), and 1 g/ml anti-CD3s (clone 145-2C11) and 1 g/ml anti-CD28 (clone 37.51) (BD Biosciences) or 10 ng/μl recombinant human IL-2 (rhIL-2, Peprotech) and 10 μg/μl MsIn₄₀₆₋₄₁₄ peptide (GQKMNAQAI, Genscript) (SEQ ID NO: 15) or 10 μg/μl GP33 peptide (KAVYNFA™, Genscript) (SEQ ID NO: 16). Splenocytes were cultured in T25 flask for overnight at 37° C., 5% C02. Cells were counted using a hemocytometer and Trypan blue and subsequently transferred into a 12 well, flat-bottom tissue-culture treated at a concentration of 5×10⁵ cells/well at 37° C., 5% C02 for 24 h prior to rAAV and CRISPR/Cas9.

rAAV serotype screening: Splenocytes from B6 mice were activated in vitro with 1 g/ml anti-CD3s (145-2C11, BD Biosciences) and 1 g/ml anti-CD28 (37.51, BD Biosciences) in the presence of 10 ng/μl recombinant human IL-2 (rhIL-2, Peprotech) and 5 ng/μl recombinant murine IL-7 (rmIL-7, R&D Systems) in T cell media at 37° C., 5% CO₂. Next, T cells were spun down and incubated with similar concentrations of various rAAV serotypes (UPenn Vector Core) engineered to express GFP. After 1 day, GFP expression in live T cells was analyzed by flow cytometry.

CRISPR/Cas9 TCR knock in of primary murine T cells and EL4 cells: At 48 h post in vitro T cell activation, primary T cells were centrifuged for 10 minutes at 200×g and 4° C. Primary T cells and EL4 cells were resuspended at 1×10⁶-1×10⁷ cells per ml in P4 solution with supplement (Lonza, V4XP-4024). Synthego sgRNAs were resuspended at 50 μM. 10× RNPs were generated by mixing Synthego sgRNAs and TrueCut Cas9 Protein v2 (ThermoFisher Scientific, A36498) at a 1:1 molar ratio and incubating at room temperature for 10 minutes. RNPs were diluted ten-fold in the cell suspension and cells were transferred to the nucleofection cuvette and incubated at room temperature for 2 minutes with the cover on. Using the Amaxa 4D Nucleofector, cells were pulsed with pulse code CM137 and allowed to rest 15 minutes in the cuvette. Cells were diluted 1:10 in prewarmed T cell recovery media (T cell media with no antibiotics) in the cuvette and allowed to recover at 37° C. for 15 minutes. T cells were transferred to pre-warmed (37° C.) T cell media containing rhIL-2 (10 ng/μl), rmIL-7 (5 ng/μl) and various concentrations of rAAV6 containing the 1045 TCR (Vigene) or 7431 TCR (Signagen) or P14 TCR (Signagen) homology donor DNA for a total of 30 minutes after nucleofection. T cells were returned to the incubator (37° C., 5% CO²) for an additional 3 days prior to flow cytometry and/or DNA sequencing analysis. Typically, both EL4 and primary T cells were 50% viable following this protocol.

Analysis of T cells in circulation from TRex animals by flow cytometry: A total of 200 l of blood was collected per animal in 20 mM EDTA in a 96-well round bottom plate. RBCs were lysed by resuspension in 150 μl ACK lysis buffer (GIBCO) for 10 minutes at room temperature. A total of 150 μl of T cell media was added to each well to quench cell lysis. Cells were spun at 350×g for 5 minutes at 4° C., the supernatant decanted, and washed 2× with 200 μl of FACS buffer (PBS+2.5% FBS). Cells were stained with a live/dead stain (Ghost, Tonbo) and murine monoclonal antibodies to CD8a (53-6.7, Biolegend) CD4 (GK1.5 BD Biosciences), and Vβ9 (MR10-2, Biolegend) to detect MsIn₄₀₆₋₄₁₄:H-2D^{b}-specific TCR.

Preparation of mononuclear cells from tissues: Spleens were mechanically dissociated to single cells. Red blood cells (RBCs) were lysed by incubation in 1 mL of Tris-ammonium chloride (ACK) lysis buffer (GIBCO) for 1-2 minutes at room temperature. 9 mL of T cell media was added to quench lysis. Cells were spun at 1400 rpm for 5 minutes at 4° C. and stored in T cell media on ice until further analyses. For PBMCs, 100 μL-200 μL of blood was collected per animal in 20 mM EDTA in a 96-well round bottom plate. RBCs were lysed by resuspension in 150 μL ACK lysis buffer (GIBCO) for 10 minutes at room temperature. 1 mL of T cell media was added to quench cell lysis. Cells were spun at 350×g for 5 minutes at 4° C., the supernatant decanted, and washed 2× with 200 μL of FACS buffer (PBS+2.5% FBS+1% NaN₃). Cells were stored in T cell media on ice prior to staining.

Flow cytometry: Mononuclear cells were stained with MsIn₄₀₆₋₄₁₄:H-2D^{b}-APC or -BV421 tetramer (1:100) in the presence of 1:100 Fc block (αCD16/32, Tonbo), and monoclonal antibodies diluted 1:100 in FACs Buffer (2% BSA+PBS) or (2.5% FBS+PBS+1% NaN₃) and specific to CD45 (30F-11, Biolegend), CD8a (53-6.7, Tonbo), CD44 (IM7, BD Biosciences), CD62L (MEL-14, Biolegend), CD69 (H1.2F3, BD Biosciences), CD25 (PC61, BD Biosciences), Vβ9 (MR10-2, Biolegend), CD3e (145-2C11, BD Biosciences), CD4 (GK1.5, BD Biosciences), and/or TCRβ (h57-597, eBiosciences) in the presence of live/dead stain (Tonbo Ghost dye in BV510 or APC ef780). Cells were fixed using Foxp3 transcription factor reagent (Tonbo), for 30 minutes at 4° C., washed and intracellular stained with aKi67 (B56, BD Biosciences) and/or Foxp3 (3G3, Tonbo) diluted 1:100 in Fix/Perm buffer (Tonbo) and stained overnight. The next day, cells were washed 2× with perm wash buffer and resuspended in FACs buffer or 0.4% PFA for 15 minutes at 4° C. Cells were resuspended in FACs buffer and Countbright Absolute Counting Beads (Thermo Fisher). Cells were acquired with a Fortessa 1770 flow cytometer and Facs Diva software (BD Biosciences). Data were analyzed using FlowJo software (version 10). ViSNE analysis was performed by gating on total live T cells with default settings of 1000 iterations, 30 perplexity and theta of 0.5 using Cytobank software.

Cell Proliferation Assay: Live mononuclear splenocytes from 1045 TRex and P14 Tg, TRex mice were counted using trypan blue and a hemocytometer. 2×10⁶ splenocytes were incubated with 5 μM Cell Trace™ Violet (CTV) (Invitrogen) diluted in PBS and incubated for 20 minutes in the dark at 37° C., 5% CO₂. Cells were washed 4× with RPMI-10 to remove excess CTV and 7.5×10⁵ CTV labeled splenocytes were plated in duplicate in 96-well round bottom plates in T cell media with 10-fold serial dilutions of gp33 or MsIn peptide ±10 ng/ul of rhIL-2. Cells were incubated in the dark for 3 days at 37° C., 5% CO₂, stained for various cell surface markers and analyzed by flow cytometry. Duplicate plates were also set up in which Golgiplug+Golgistop (BD Biosciences) were added for 5 hours prior to cell surface staining and intracellular staining for IFNγ as described above. Data was acquired in the Center for Immunology on a Fortessa 1770 or Fortessa X-20 and analyzed using FACs Diva software (BD Biosciences).

Intracellular cytokine staining: Splenic mononuclear cells were activated in vitro with MSLN peptide or anti-CD3+ anti-CD28 as described above. On day 6, 1×10⁵ activated T cells were centrifuged and resuspended with congenic (CD45.1+) peptide-pulsed splenocytes at a 1:5 T cell to APC ratio. To assess functional avidity, we titrated MsIn406-414 or gp33 peptide (Genscript). Cells were incubated in round-bottom 96-well plates in a total volume of 200 of T cell media+Golgiplug and Golgistop (BD Biosciences) for 5 hours at 37° C., 5% CO₂. Cells were subsequently stained in the presence of live/dead stain (Tonbo Ghost dye) with cell surface antibodies including CD45.1, to exclude APCs (A20, Biolegend, San Diego, CA), as well as CD45 (30F-11, Biolegend), CD8a (53-6.7, Tonbo), CD4 (GK1.5, BD Biosciences), CD44 (IM7, BD Biosciencs) and others described above diluted 1:100 in FACs Buffer (PBS+2.5% FBS+NaN₃) and incubated for 30 minutes in the dark at 4° C. Cells were washed 2× with FACs buffer, fixed and permeabilized (BD Biosciences Fixation Kit) and incubated with antibodies specific IFNγ (XMG1.2, Biolegend), TNFα (MP6-XT22, Biolegend) and IL-2 (JESH-65H4, Biolegend) diluted 1:100 in permeabilization buffer overnight in the dark at 4° C. Cells were washed 2× and resuspended in FACs buffer and collected using a Fortessa 1770 and FACSDiva™ software (BD Biosciences).

Cell numbers normalized to tissue gram: The number of live CD45+ cells collected per tube was determined using FlowJo analysis software and the equation: #CD45+ cells per tube (n)=(#Beads/ #Cells)×(Concentration of beads×Volume of beads added). Total number of cells collected from the entire single cell suspension was determined by multiplying n by total number of stains. Cell numbers were normalized to total spleen.

ViSNE analysis: ViSNE analysis was performed by gating on total live T cells with default settings of 1000 iterations, 30 perplexity and theta of 0.5 using Cytobank software.

MsIn406-414 tetramer production: H-2Db-restricted biotinylated monomer was produced by incubating MsIn₄₀₆₋₄₁₄ peptide with purified H-2Db and B2m followed by purification via Fast Protein Liquid Chromatography system (Aktaprime plus, GE health care) similar to as described (24). Biotinylated monomer was conjugated to streptavidin R-APC or R-BV421 (Invitrogen) to produce fluorscent MsIn₄₀₆₋₄₁₄/H-2Db tetramer. To detect TRex CD8 T cells binding, single cell suspensions of splenocytes were stained with tetramer (1:100) for 45 minutes on ice.

Immunofluorescence: Tissues were embedded in OCT (Tissue-Tek) and stored at −80° C. 7 μm sections were cut using a Cryostat and fixed in acetone at −20° C. for 10 min. Sections were rehydrated with PBS+1% bovine serum albumin (BSA) and incubated for 1 hr at rt with primary antibodies to rat anti-mouse MsIn (MBL, B35, 1:100) diluted in PBS+1% BSA. Slides were washed 3× in PBS+1% BSA and incubated with anti-rat AF546 (Invitrogen, 1:500) for 1 hr at rt in the dark. Stained slides were then washed 3× with PBS+1% BSA, washed 3× with PBS, and mounted in DAPI Prolong Gold (Life Technologies). Images were acquired on a Leica DM6000 epifluorescent microscope at the University of Minnesota Center for Immunology using Imaris 9.1.0 (Bitplane).

Statistical Analysis: Statistical analyses were performed using Prism (version 7.0). All mouse experiments reflect n=3-12 mice per group. Unpaired, two-tailed student's T test was used to compare 2-group data. One-way ANOVA and Tukey post-test were used for multiple comparisons. Data are presented as mean±standard error of the mean (S.E.M.) and p<0.05 was considered significant. *, p<0.05; **p<0.005; ***, p<0.0005.

### Example 2—Characterization of TCR Replacement with MsIn TCRs

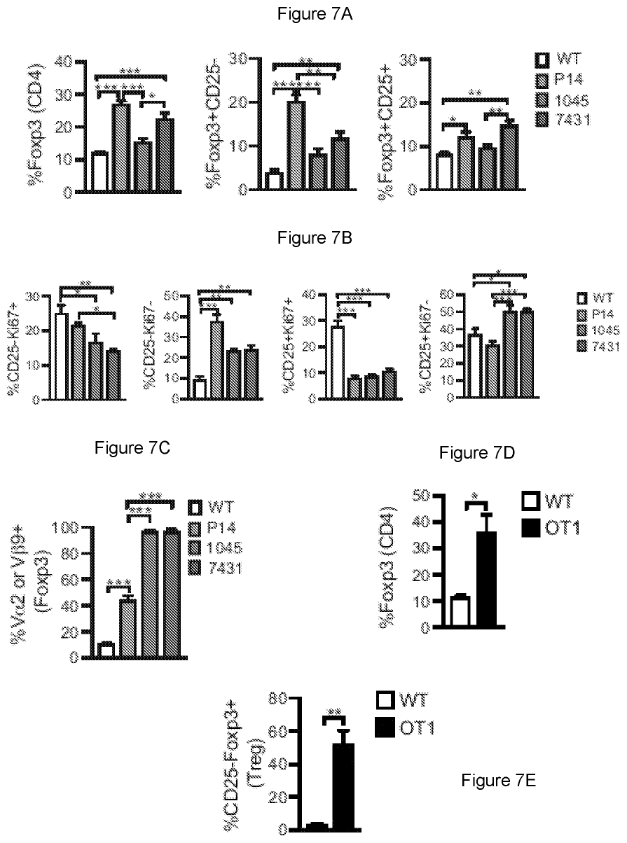

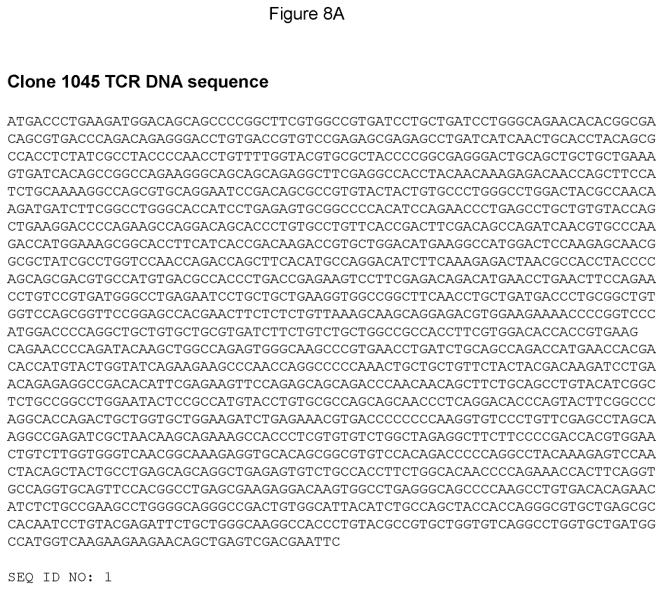

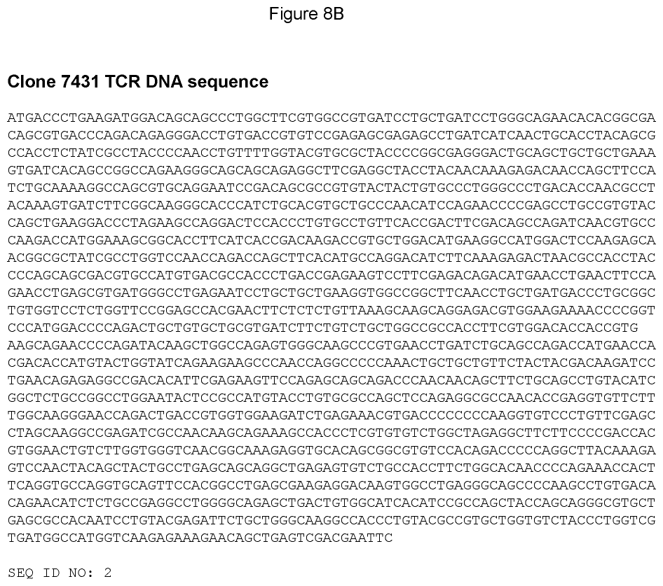

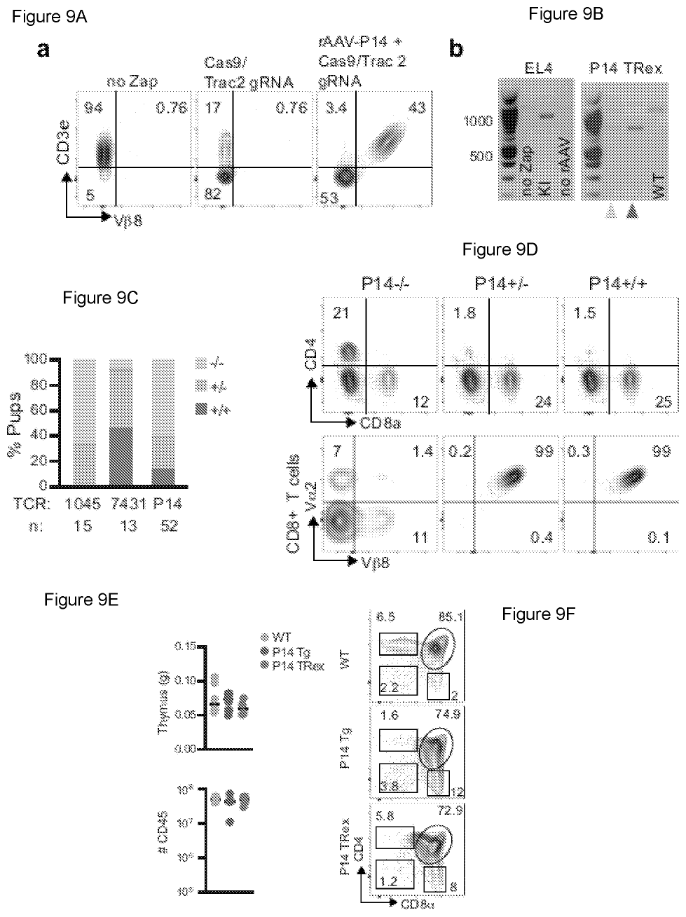

Murine MsIn₄₀₆₋₄₁₄:H-2D^{b}-specific TCRs for adoptive cell therapy were previously cloned and expressed (2). The 1045 TCR was the highest affinity TCR obtained from MsIn^{-/-} mice and the 7431 TCR was the highest affinity TCR obtained from wild type mice. The sequences of the 1045 and 7431 TCR are set out in FIG. 7. Both TCRs utilized Vα4 and V39 and differed only in CDR3 sequence (2), which determines antigen binding and TCR specificity (13). Due to limitations of previous TCR engineering approach that required retroviral transduction, random TCR integration, the dependency on P14 T cells for exogenous TCR expression, and poor T cell viability, it was desired to develop mouse models to study physiologic MsIn-specific T cells in cancer.

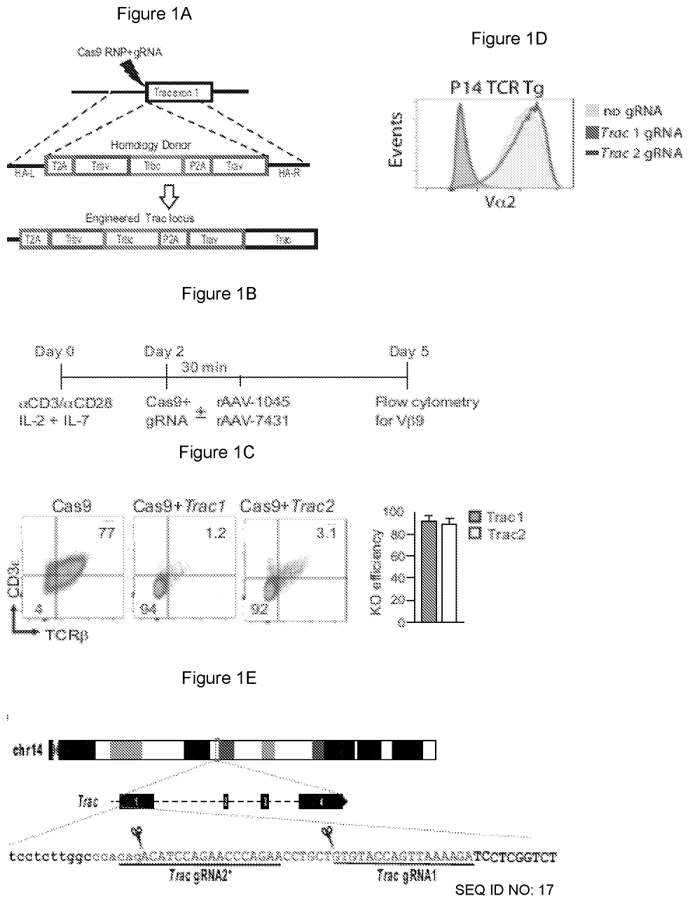

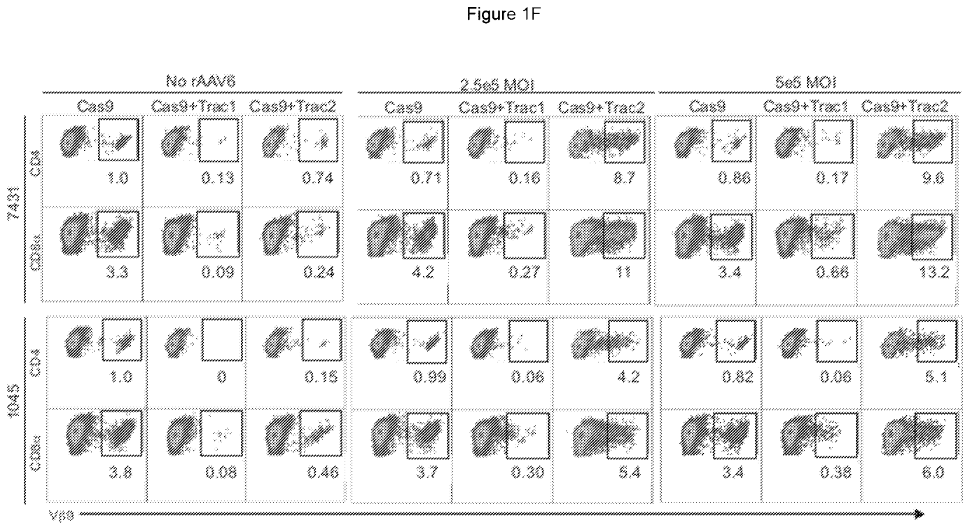

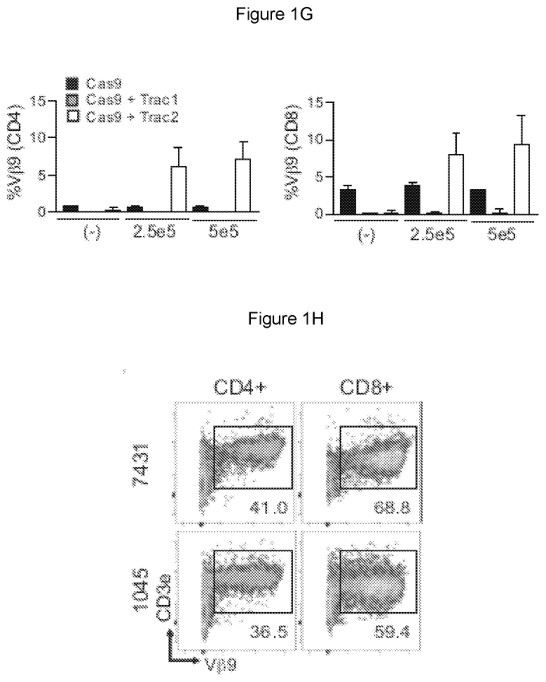

Targeting MsIn-specific TCRs to Trac in primary murine T cells: First, a panel of rAAV-GFP serotypes was screened to identify one that was efficient at infecting mouse T cells. Similar to human T cells (14), rAAV6 infected ˜20-35% of the activated primary mouse T cells, without negatively influencing T cell viability. Codon optimized 1045 or 7431 TCRβ variable (V), TCRβ Constant (C) and TCRα V were synthesized, linked by a self-cleaving P2A element (15) for coordinated gene expression (FIG. 1A). The TCR sequences were flanked by ˜400 bp homology arms (HA) encoding endogenous murine Trac sequences and cloned into rAAV6 (FIG. 1A). Next, two murine Trac-specific guide RNAs (gRNAs) specific to Trac exon 1 (Trac gRNA 1) or directly 5′ to Trac exon 1 (Trac gRNA 2) complexed to Cas9 ribonucleoprotein (RNP) were nucleofected into activated splenic polyclonal T cells as show in FIG. 1B, using an optimized protocol previously described (16). Both Trac gRNAs caused cell surface loss of TCR and CD3 in >90% of activated polyclonal T cells (FIG. 1C). In contrast, only Trac gRNA 1, but not Trac gRNA 2, disrupted TCR expression in P14 TCR transgenic T cells as determined Vα2 staining (FIG. 1D). Thus, gRNA1 cut within Trac exon1, thereby disrupting both transgenic and endogenous TCR expression, whereas gRNA2, which targeted the noncoding region upstream of Trac exon 1 (FIG. 1E) reduced endogenous but not transgenic P14 TCR expression, highlighting the specificity of this approach.

The efficiency of donor TCR expression in polyclonal in vitro activated T cells using the protocol shown in FIG. 1B was tested. The proportion of T cells that express V39, which is the Trbv chain utilized by both 1045 and 7431 TCRs was measured (2). As expected, Cas9 complexed with Trac gRNAs with rAAV reduced Vβ9 in both CD4 and CD8 T cells (FIG. 1F). Trac gRNA 2, but not gRNA 1, resulted in the integration of the donor TCRs in 5-15% of T cells at both rAAV concentrations tested (FIG. 1F-G). To assess TCR functionality, the T cells were restimulated with peptide-pulsed irradiated syngeneic splenocytes and analyzed the frequency of Vβ9+ T cells 5 days later. A marked enrichment in Vβ9+ T cell frequency that ranged from 5-10% prior to restimulation to 38-70% following antigen was observed, which corresponded to a 5-fold increase in T cell number (FIG. 1H). Thus, an approach engineer murine polyclonal T cells with physiologically expressed tumor-reactive TCR by replacement of the endogenous TCR was developed.

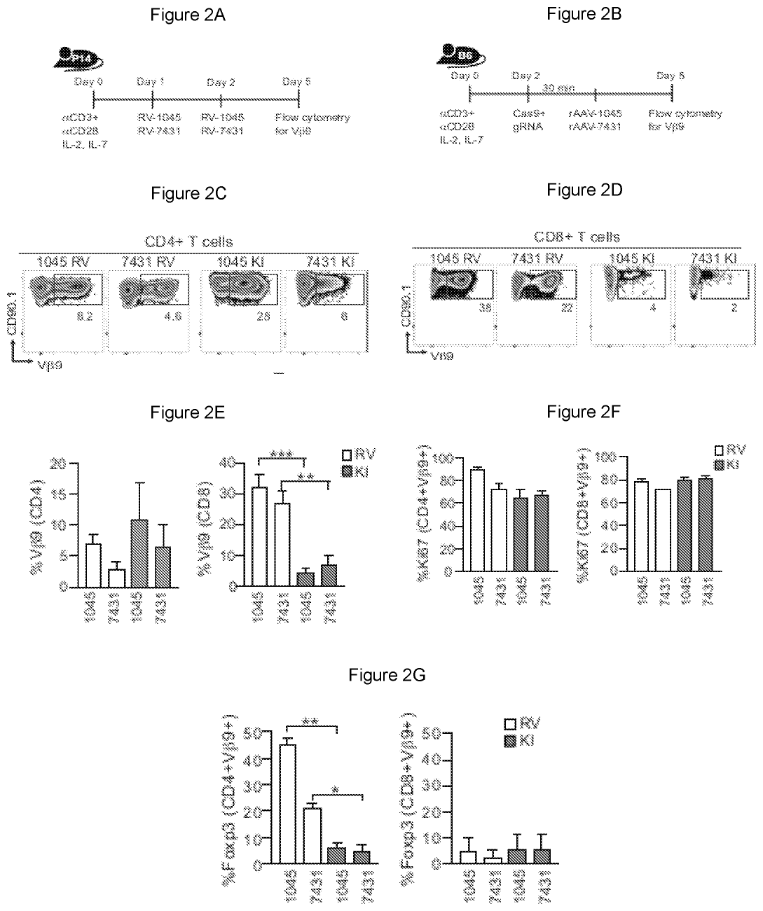

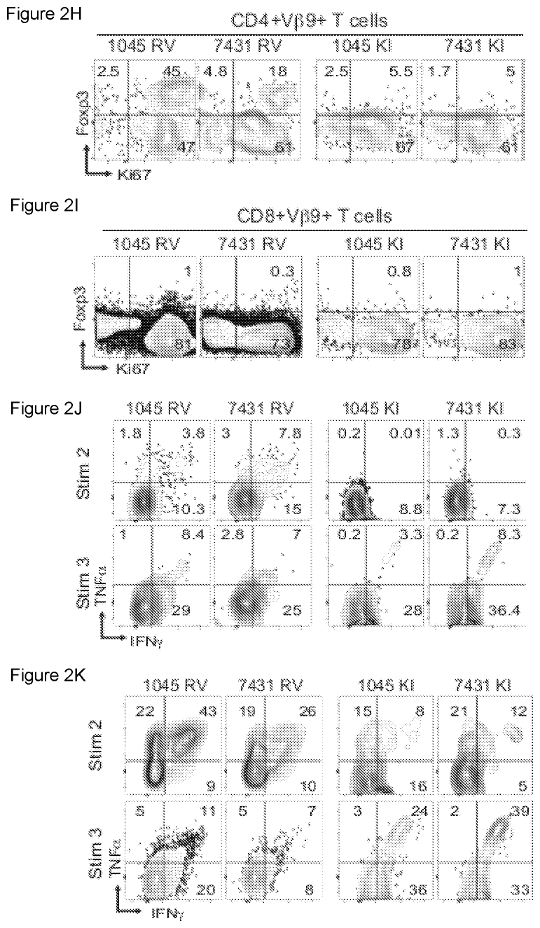

Targeting TCRs into Trac sustains engineered T cell function and obviates Treg expansion: Efficiency of retroviral transduction (RV) of P14 T cells (FIG. 2A) was compared to the CRISPR/Cas9 and rAAV-mediated TCR Trac knock-in (KI) of polyclonal T cells (FIG. 2B). P14 T cells were previously relied on for T cell engineering because exogenous TCRs readily outcompete the P14 transgenic TCR but not polyclonal TCRs for cell surface expression (2). Efficiency of donor TCR expression in CD4 T cells was not significantly different among RV and KI approaches (FIG. 2C). In contrast, RV transduction significantly increased the frequency of CD8 T cells that expressed the 1045 or 7431 TCR as compared to the KI approach (FIG. 2D-E). Proliferation of the genetically modified Vβ9+ T cells was similar among the two approaches (FIG. 2F). Unexpectedly, RV increased the expansion of TCR engineered CD4+ Foxp3+ Treg which was not observed with the TCR KI approach (FIG. 2G-H). Foxp3 was not expressed in TCR engineered CD8 T cells (FIG. 2G, 2I). Thus, the prior studies of RV of P14 T cells with a MsIn-specific TCR for adoptive cell therapy likely contained infused TCR engineered, tumor-reactive CD4+ Treg (2, 11, 12), which may have limited antitumor activity.

Cytokine production was measured by RV and KI T cells following repetitive in vitro stimulations with antigen. Few TCR engineered CD4+ T cells produced cytokines as the donor TCRs are MHC class I restricted (FIG. 2J). While a higher frequency of RV transduced Vβ9+CD8 T cells produced cytokines following the 2^{nd} stimulation compared to KI Vβ9+ T cells (FIG. 2K), a higher frequency of KI Vβ9+ CD8 T cells produced cytokines following the 3^{rd} in vitro stimulation compared to RV Vβ9+ CD8 T cells (FIG. 2K). Vβ9 mean fluorescence intensity (MFI) cells exhibited variability among independent experiments and was not significantly different between the 2 approaches. Thus, the KI approach appeared advantageous because it permits TCR engineering of polyclonal T cells, obviates Treg expansion and results in physiologic TCR expression which may improve T cell functionality during recurrent antigenic exposure. However, limitations of the KI approach were the low efficiency of TCR expression, and similar to RV approach, necessitated in vitro expansion and differentiation into effector T cells. Both approaches required the in vitro differentiation and expansion of effector T cells precluding studies of naïve MsIn-specific T cells.

### Example 3-Generation and Characterization of MsIn TCR Knock-In Mice

To create a standardized and reproducible source of naïve murine MsIn-specific T cells, the above approach was adapted in zygotes to create MsIn-specific TCR_exchange (TRex) mice.

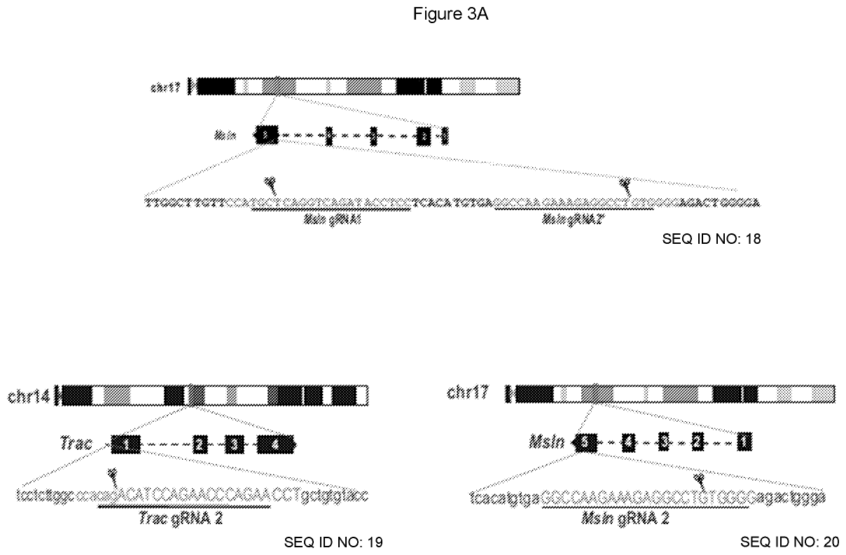

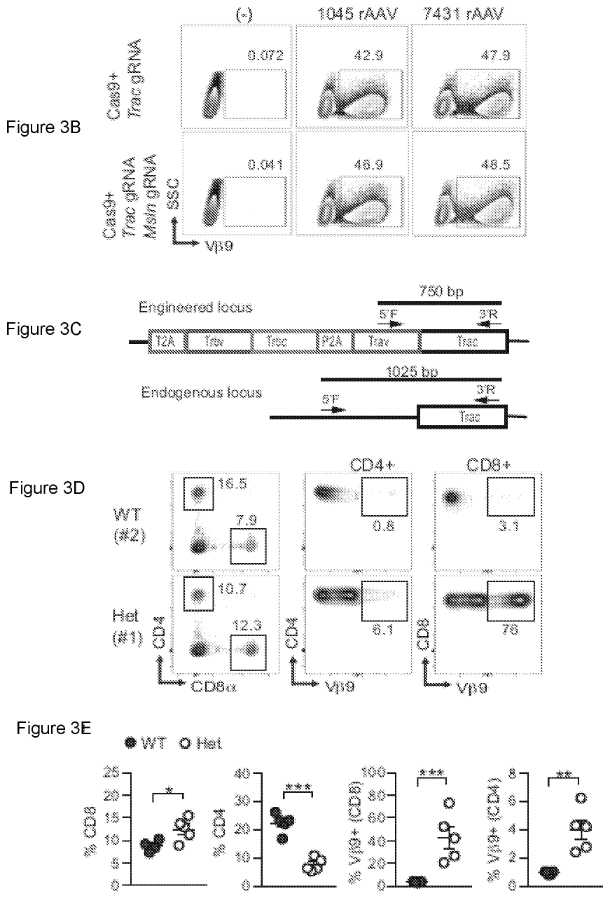

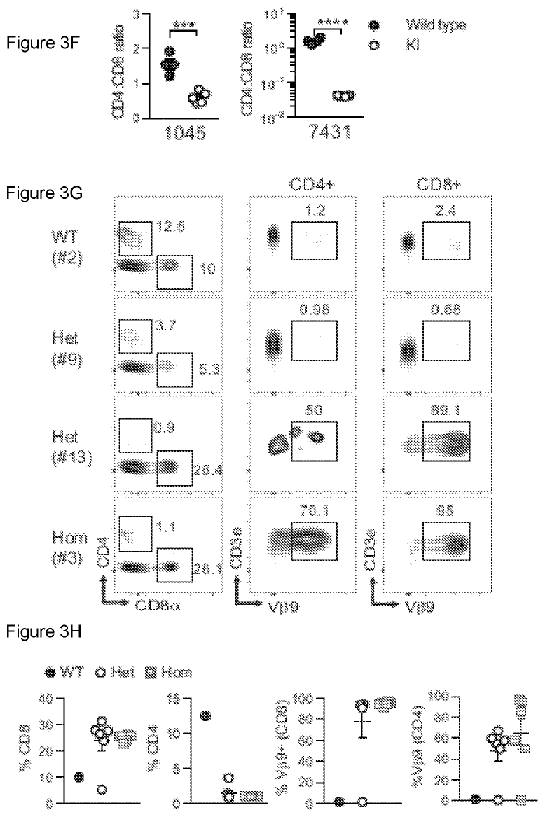

Rapid generation of MsIn-specific TCR exchange (TRex) mice: MsIn TCR KI mice were generated by targeting MsIn-specific TCRs to the Trac locus. MsIn may promote T cell tolerance (17) because it is expressed at low levels in normal tissues (3). To circumvent this, 2 murine MsIn-specific gRNAs complexed to Cas9 RNP specific to target murine MsIn exon 4 were designed and tested (FIG. 3A). Both gRNAs induced indel rates >80% of 3T3 cells as determined by PCR amplification, Sanger sequencing, and Interference of Crispr Edits (ICE) analysis. Both the 1045 and 7431 TCRs integrated in over 40% of EL4 cells regardless of co-nucleofection with Cas9 RNP+MsIn gRNA 2 (FIG. 3B). DNA sequence analysis validated the specific targeting of both the Trac and MsIn genes in EL4 cells. Therefore, a CRISPR-READI approach (18) was adapted to integrate the 1045 or 7431 TCRs into the Trac locus while concurrently knocking out MsIn in zygotes. A junction PCR was developed to amplify both the integrated donor TCR and the wild type Trac locus (FIG. 3C). 5/15 pups integrated the 1045 TCR in one Trac allele. Normal proportions of CD4 and CD8 T cells were detected and a low frequency Vβ9+ T cells (˜1-3%) in the 10 pups that were 1045 negative by PCR (FIG. 3D-3E). All 5 animals that were 1045 positive by PCR had increased circulating CD8 T cells and decreased circulating CD4 T cells (FIG. 3E) resulting in a lower CD4:CD8 T cell ratio (FIG. 3F) and consistent with forced expression of an MHC class I-restricted TCR (10, 19). In the 1045+ mice, 70-80% of the CD8 T cells expressed Vβ9 (FIG. 3G and Table 1) and CD4 T cells were also enriched for Vβ9 (FIG. 3G and Table 1). Multiple indels in MsIn exon 4 were detected in 9/15 animals and in all (5/5) of the 1045 pups (Table 1). In 3 of the 1045 animals (Table 1), indels were detected in only one allele and yet CD8+Vβ9+ T cells remained enriched in the periphery indicating central tolerance of 1045 T cells is incomplete (Table 1).

TABLE 1
Analysis of T cells and MsIn locus from 1045 pups

| | | | | | % Vβ9 | | % Vβ9 | % MsIn | MsIn |
|---|---|---|---|---|---|---|---|---|---|
| # | ID | Sex | 1045^{&} | % CD8^{Φ} | (CD8) | % CD4 | (CD4) | KO^{#Φ} | [symbol] In/Dels |
| 1 | 3290 | M | +/− | 13 | 73.3 | 10.7 | 6.22 | 84% | +1/−7 |
| 2 | 3291 | M | −/− | n.d. | n.d. | n.d. | n.d. | 50% | −3/−8 |
| 3 | 3292 | M | −/− | n.d. | n.d. | n.d. | n.d. | 0% | none |
| 4 | 3293 | M | −/− | n.d. | n.d. | n.d. | n.d. | 11% | +1 |
| 5 | 3294 | M | +/− | 13.7 | 52.4 | 6.09 | 4.19 | 93% | +4/+1/− [symbol] /−7 |
| 6 | 3295 | M | +/− | 11.3 | 26.7 | 5.23 | 2.75 | 10-50% | n.r. |
| 7 | 3296 | F | −/− | n.d. | n.d. | n.d. | n.d. | 0% | none |
| 8 | 3297 | F | −/− | n.d. | n.d. | n.d. | n.d. | 90% | +13/+1 |
| 9 | 3298 | F | −/− | n.d. | n.d. | n.d. | n.d. | 0% | none |
| 10 | 3299 | F | −/− | n.d. | n.d. | n.d. | n.d. | 0% | none |
| 11 | 3300 | F | +/− | 15.5 | 20.5 | 9.04 | 4.29 | 93% | −23 |
| 12 | 3301 | M | +/− | 8.91 | 42.3 | 6.29 | 2.39 | 40-50% | +1/−1/−2 |
| 13 | 3302 | M | −/− | n.d. | n.d. | n.d. | n.d. | n.r. | n.r. |
| 14 | 3303 | M | −/− | n.d. | n.d. | n.d. | n.d. | 45% | −1/−2 |
| 15 | 3304 | M | −/− | n.d. | n.d. | n.d. | n.d. | 47% | +1 |

^{&}1045 knock-in was determined by junction PCR of tail DNA
^{Φ}n.d., not determined; n.r., no results indicating sequence analysis was attempted but data were inconclusive.
^{#}MsIn knockout was determined PCR amplification of MsIn exon 1 followed by Sanger sequencing and Inference of Crispr Edits (ICE) analysis software (Synthego) [symbol]
indicates data missing or illegible when filed

Next, zygotes were engineered using the lower affinity 7431 TCR with similar methods as the 1045 animals. Strikingly, PCR analysis showed 12/13 (92%) of the pups were 7431+ with 5/13 (38%) homozygous and 7/13 (54%) heterozygous for 7431. Again, circulating T cells were significantly biased toward CD8 T cells at the expense of CD4 T cells in the 7431 animals (FIG. 3G-H, FIG. 3F). There was a dramatic reduction in total circulating T cells and a complete lack of Vβ9+ T cells in one of the 7431 animals (FIG. 3G). It was noted that an additional band between the WT and KI bands appeared in this mouse suggesting CRISPR/Cas9 cut Trac, but the 7431 TCR failed to integrate correctly (FIG. 3G). In the 7431+ mice, CD4 and CD8 T cells co-expressed CD3 and Vβ9 (FIG. 3G-H, Table 2), consistent with the PCR results, and correct 7431 TCR integration. All the 7431+ animals exhibited several indels in MsIn exon 4 with knockout scores indicative that MsIn was targeted on both alleles (Table 2).

TABLE 2
Analysis of T cells and MsIn locus from 7431 pups

| | | | | | % Vβ9 | | % Vβ9 | % MsIn | MsIn |
|---|---|---|---|---|---|---|---|---|---|
| # | ID | Sex | 7431^{&} | % CD8^{Φ} | (CD8) | % CD4 | (CD4) | Indel^{#Φ} | Indel |
| 1 | 3291 | F | +/− | 27.7 | 92.3 | 0.87 | 55.3 | 86* | −2, +4, −19 |
| 2 | 3292 | F | −/− | 9.98 | 2.31 | 12.5 | 1.24 | 0 | none |
| 3 | 3293 | F | +/+ | 26.1 | 94.9 | 1.11 | 70.1 | 97* | −7 |
| 4 | 3294 | F | +/− | 31.3 | 92.6 | 1.0 | 59.8 | 97* | −4 |
| 5 | 3295 | F | +/− | 23.9 | 91.7 | 1.4 | 67 | 84* | −8 |
| 6 | 3296 | M | +/+ | 25.7 | 74.4 | 1.1 | 95.5 | 91* | −5, +1 |
| 7 | 3297 | M | +/− | 27.9 | 91.6 | 1.9 | 57.9 | 95 (KO 47) | −7, −9 |
| 8 | 3298 | M | +/+ | 24.1 | 91 | 1.0 | 64.2 | 95* | −1, −8 |
| 9 | 3300 | M | +/−^{Ψ} | 5.25 | 0.98 | 3.67 | 0.68 | 86* | +1, −8 |
| 10 | 3301 | M | +/+ | 25.4 | 93.7 | 0.97 | 69 | 97* | −8 |
| 11 | 3302 | M | +/+ | 25.7 | 96.2 | 1.0 | 73 | 92 (KO 45) | −1, −6 |
| 12 | 3303 | M | +/+ | 22.0 | 92.8 | 0.96 | 73.5 | 96* | +1 |
| 13 | 3304 | M | +/− | 26.4 | 88.8 | 0.89 | 50 | 97* | −4 |

^{&}7431 knock-in was determined by junction PCR of tail DNA from pups
^{Φ}n.d., not determined; n.r., no results indicating sequence analysis was attempted but data were inconclusive.
^{#}MsIn knockout was determined PCR amplification of MsIn exon 1 followed by Sanger sequencing and Inference of Crispr Edits (ICE) analysis software (https://www.synthego.com/products/bioinformatics/crispr-analysis).
*Knockout score was identical to % Indel
^{Ψ}An additional band was detected between the WT and KI
*Knockout score was identical to % Indel

To test if MsIn protein is disrupted in pups from founders, lung from wild type (WT) and MsIn TCR-exchanged (TRex) mice was stained which exhibited indels in both MsIn alleles. MsIn was detected in WT lung but not in lungs from 7431 or 1045 mice homozygous for MsIn indels (e.g., MsIn−/−). Thus, an efficient method to replace endogenous TCRs with a TCR of desired antigen specificity while concomitantly disrupting target gene expression was established.

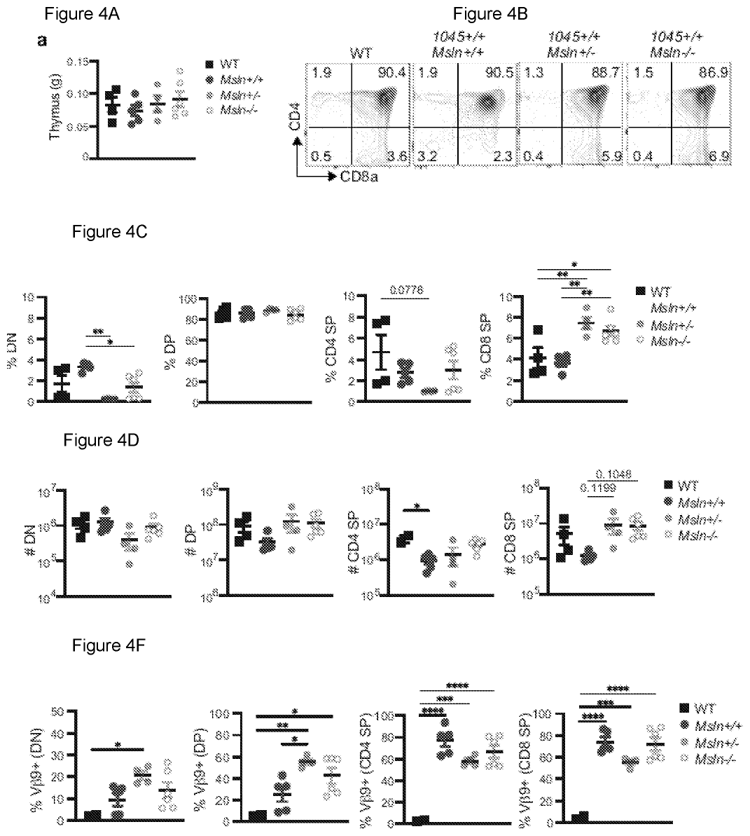

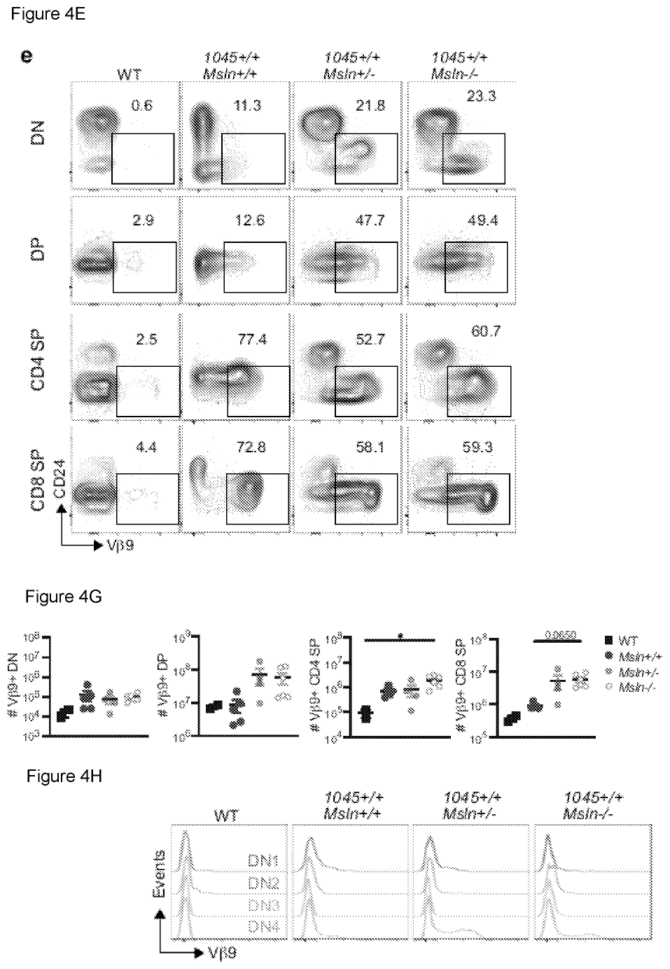

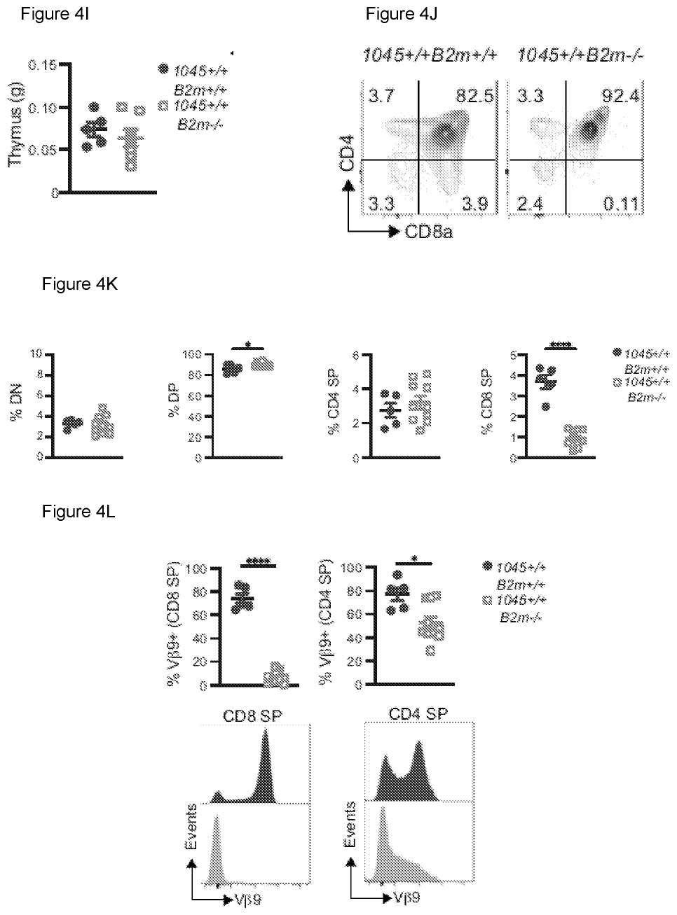

High affinity MsIn-specific T cells undergo central tolerance in a MsIn dose dependent manner: To investigate T cell development in TRex mice, 1045 TRex #11 were backcrossed onto MsIn^{WT/WT}, MsIn^{WT/-23} and MsIn^{-23/-23} background (referred to as MsIn+/−, MsIn^{+/-}, and MsIn^{-/-}, respectively, Table 1) and analyzed thymocytes in 1045^{+/+} offspring. Thymus weight (FIG. 4A) and CD45+ cell number were similar regardless of MsIn status. Thymocyte maturation occurs through sequential differentiation program that is distinguished by CD4 and/or CD8 coreceptor expression (17). The earliest thymocyte progenitors lack CD4 and CD8 (double negative, DN) that differentiate into CD4+ CD8+ double positive (DP) followed by maturation into CD4 or CD8 single positive (SP) cells. The frequency and number of DNs and DPs were similar among the strains (FIG. 4B-D). In 1045^{+/+} MsIn^{+/-} and MsIn^{-/-} TRex mice, thymocytes were biased toward CD8 SP (FIG. 4B-D). CD8 SP frequency and number was significantly reduced in MsIn^{+/+} vs. MsIn^{+/-} and MsIn^{-/-} 1045 TRex mice (FIG. 4C-D) supporting the premise that negative selection to MsIn is gene dose dependent. Vβ9 was increased in most thymocyte stages in TRex vs. WT mice (FIGS. 4E-F) and Vβ9+ thymocytes downregulated CD24, consistent with maturation (FIG. 4E). Vβ9+DP and Vβ9+ CD8 SP number trended to be reduced in MsIn^{+/+} vs. MsIn^{+/-} and MsIn^{-/-} 1045^{+/+} TRex mice (FIG. 4G), again supporting tolerance to MsIn is gene dose dependent.

The DN stage is further subdivided into DN1-DN4 based on CD25 and CD44 expression (Godfrey et al., J. Immunol. 150, (1993)). TCRP and TCRα chains undergo a highly ordered and sequential rearrangement in which TCRP is rearranged at DN3 (17). Rapid cell proliferation and TCRα upregulation occurs in the transition from DN4 to DP stage and results in functional aR TCR heterodimers on DP cells (Koyasu, et al., Int. Immunol. 9, (1997)). Since the 1045 TCR is integrated into Trac in TRex mice, it is expected that the donor TCR would be detectable at the DN4 stage. As such, V39 was first detected at the DN4 stage cells in 1045 TRex mice (FIG. 4H), supporting physiological TCR regulation and maturation in TRex mice.

To assess if MHC I is required for T cell development, 1045^{+/+} TRex alleles were backcrossed to the B2m^{-/-} background, which lack functional MHC I (Koller, et al., J. Immunol. 184, (2010)). Thymus weight trended to be smaller in 1045^{+/+} B2m^{-/-} vs. B2m^{+/+} mice (FIG. 4I). CD8 SP frequency was dramatically reduced in 1045^{+/+} B2m^{-/-} vs. B2m^{+/+} TRex mice (FIGS. 4J-K) and a compensatory increase in DP frequency in 1045^{+/+} B2m^{-/-} mice was observed (FIG. 4J-K). CD8+Vβ9+ and CD4+Vβ9+ T cell frequency were reduced in 1045^{+/+} B2m^{-/-} TRex mice (FIG. 4L), supporting the premise that MHC I is required for positive selection. Thus, T cells appear to develop normally in TRex mice.

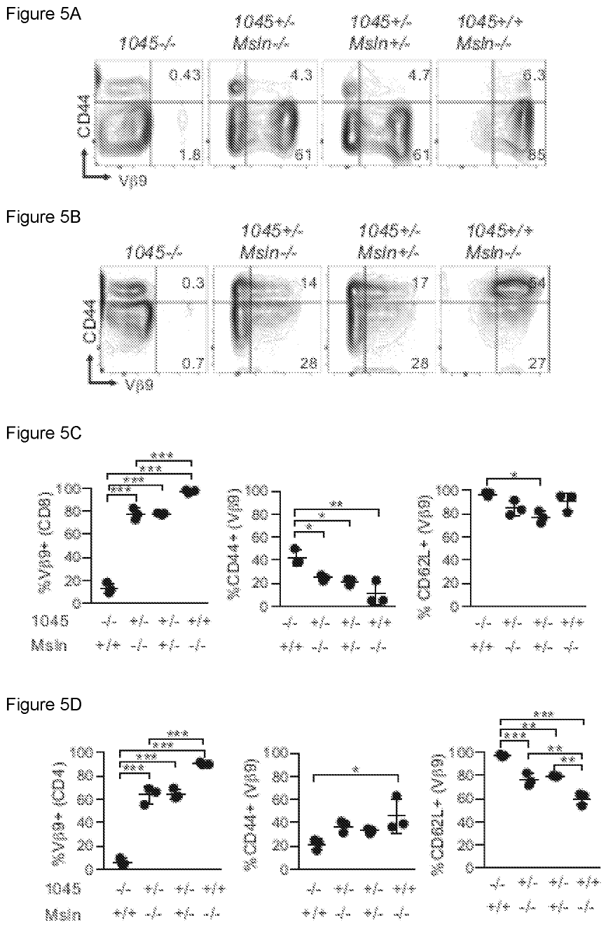

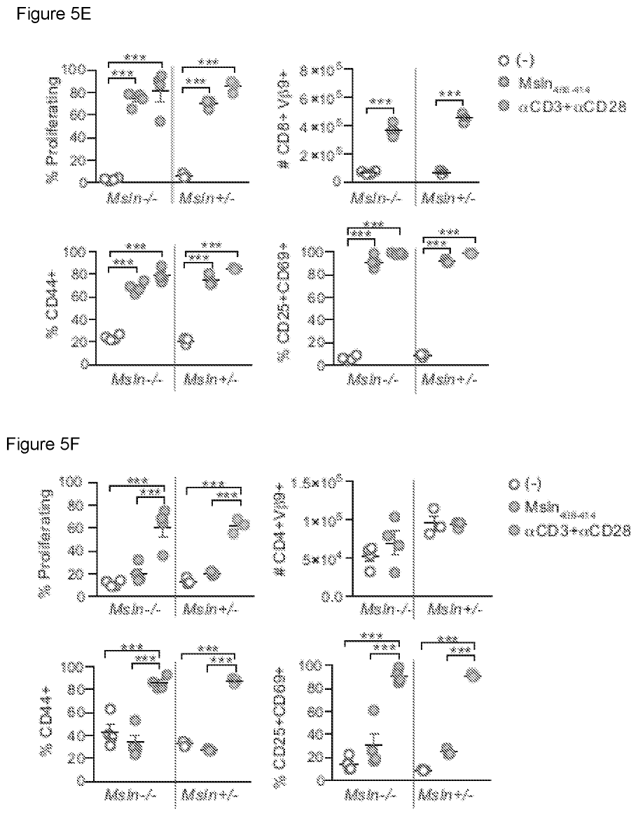

Peripheral 1045 TRex T cells are functional in MsIn^{+/-} and MsIn^{-/-} mice: To investigate the functionality of T cells from 1045 TRex mice, 1045 mouse #11 were bred onto MsIn^{WT/WT}, MsIn^{WT/-23} and MsIn^{-23/-23} background (the latter referred to as MsIn^{-/-}, Table 1). Consistent with blood from founders, T cells were biased toward the CD8 T cell lineage in 1045 MsIn WT and MsIn^{-/-} TRex mice (not shown). Most splenic CD8 (FIG. 5A) and CD4 (FIG. 5B) T cells expressed the 1045 TCR irrespective of MsIn indicating that the 1045 TCR was germline and MsIn did not appear to interfere with 1045 T cell development. Significantly more T cells expressed Vβ9 from 1045 homozygous compared to 1045 heterozygous TRex mice (FIG. 5C, D). Most CD4 and CD8 Vβ9+ T cells had a CD44^{low}CD62L⁺ naïve phenotype irrespective of MsIn status (FIG. 5C, D). In contrast, a higher frequency of CD4+Vβ9+ T cells upregulated CD44 and downregulated CD62L in 1045^{+/+} vs. 1045^{+/-} MsIn^{-/-} mice (FIG. 5D), a phenotype that was independent of self-antigen recognition.

To determine if Vβ9+ T cells recognized antigen, splenocytes from 1045 TRex mice were labeled with a proliferation dye, incubated with MsIn₄₀₆₋₄₁₄ and quantified proliferation and T cell activation 3 days later. Splenic CD8+Vβ9+ proliferated and upregulated TCR signaling molecules CD25 and CD69 in response to MsIn₄₀₆₋₄₁₄-pulsed APCs (FIG. 5E). In contrast, rare CD8+V39− T cells from TRex mice failed to respond to MsIn but were activated following a non-specific αCD3+αCD28 stimulation (FIG. 5E). Presence of a single MsIn allele did not impact 1045 T cell functionality in vitro (FIG. 5E), consistent with a lack of an overt role for MsIn inducing 1045 T cell tolerance. CD4+Vβ9+ T cells upregulated CD69 following MsIn₄₀₆₋₄₁₄:H-2D^{b} recognition yet only moderately proliferated (FIG. 5F). These data suggest that CD8 coreceptor binding MHC class I is likely required for full 1045 T cell function (20). Thus, T cells that express a high affinity MsIn-specific TCR appear highly functional despite self-antigen expression.

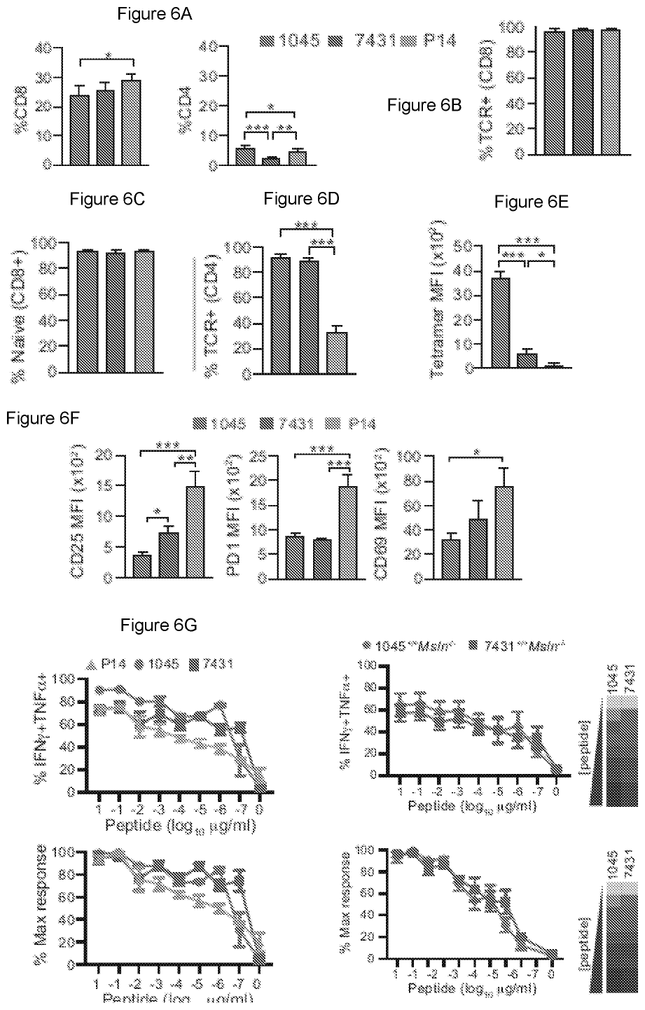

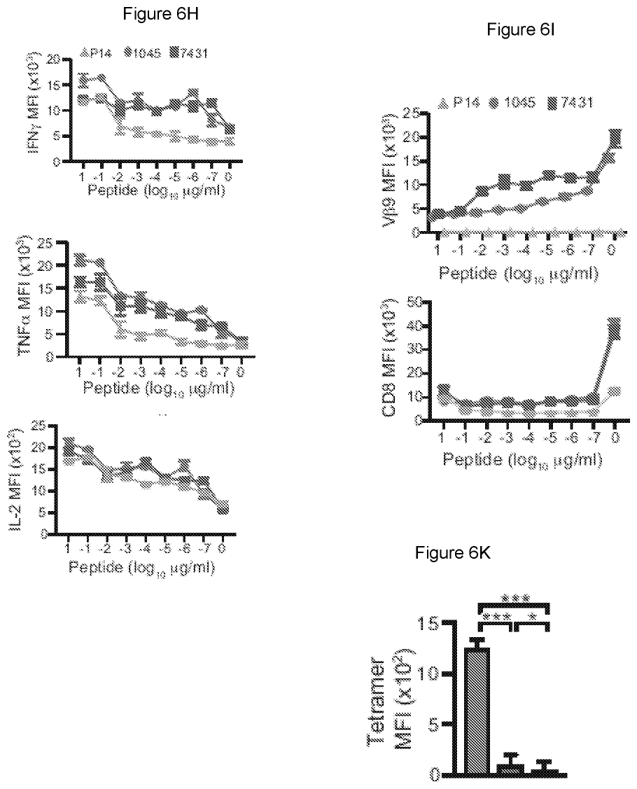

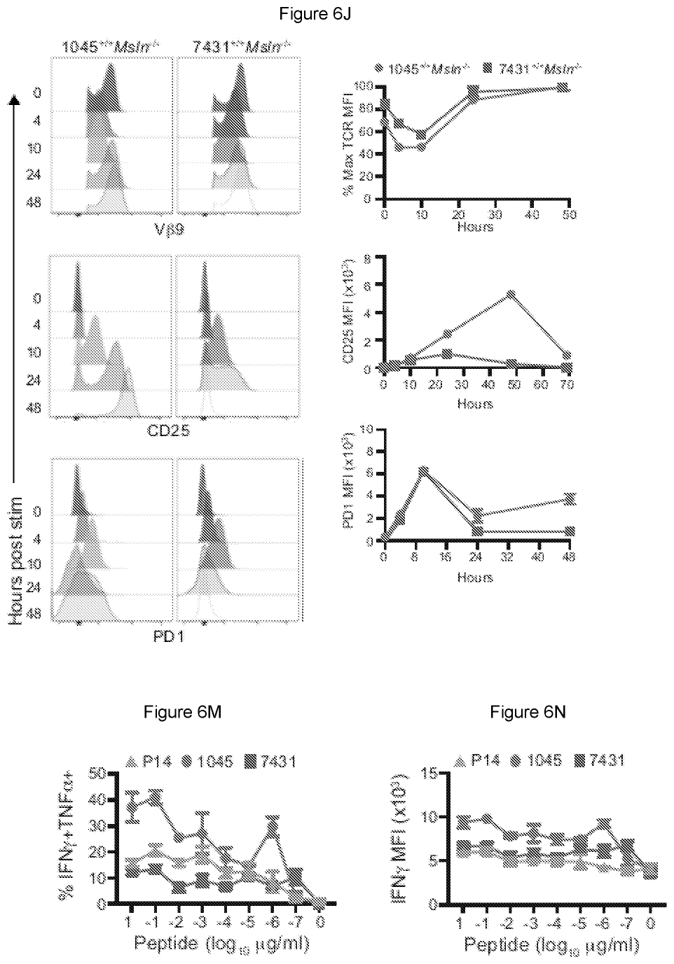

T cells from 7431 and 1045 TRex MsIn^{-/-} animals were assessed by comparing to P14 TCR transgenic T cells. Spleen weight and cellularity were similar among the 3 cohorts and T cells were biased toward the CD8 lineage (FIG. 6A). A higher frequency (FIG. 6A) and number (FIG. 5A) of CD4+ T cells in 1045 mice was noted. Over 95% of CD8 T cells expressed the MsIn-specific TCRs in both 7431 and 1045 TRex mice (FIG. 6B). Both 7431 and 1045 T cells exhibited a broader spectrum of cell surface TCR as compared to P14 T cells (FIG. 6B). Splenic CD8 T cells exhibited a naïve (CD44-CD62L+) and resting (CD25−Ki67−) phenotype in all three strains (FIG. 6C). Over 90% of CD4 T cells expressed the MsIn-specific TCR in 1045 and 7431 mice (FIG. 6D). In contrast, only 30-40% of CD4 T cells expressed the gp33-specific TCR in P14 mice (FIG. 6D). As the CRISPR KI approach caused indels in Trac, donor TCR is likely required for CD4 T cell maturation in the TRex mice whereas in P14 transgenic mice, CD4 T cells can express endogenous TCRs.

As Tregs accumulate during in vitro expansion of P14 Tg T cells, Tregs from P14 Tg mice were compared to the 1045 and 7431 MsIn^{-/-} TRex strains. Tregs were disproportionately enriched among CD4 T cells from P14 Tg compared to WT or TRex mice. Tregs were biased toward a CD25-Foxp3+ subset in P14 mice, which may represent precursors to CD25+ Foxp3+ Treg (31, 32), and were more proliferative. In contrast to T cells from TRex and WT mice, T cells from P14 mice activated with αCD3+αCD28 and IL-2 exhibited increased frequency of Foxp3+ CD25+ Tregs, and many of these did not express Vα2, the P14 TCRα chain. Thus, endogenous TCR expression appears to not be the only factor contributing to disproportionate Treg accumulation in P14 transgenic mice. Additionally, a higher proportion of CD4 T cells were Treg in OT1 TCR transgenic (19) compared to WT mice. Thus, the TRex approach may overcome some Treg abnormalities in traditional TCR transgenics.

TCR Trac targeting improves the functional avidity of a low affinity TCR. Next, the functionality of 7431^{+/+} and 1045^{+/+} T cells from MsIn^{-/-} TRex animals was analyzed. Spleen weight, CD45+ cell number, and a bias toward the CD8 lineage (FIG. 6A) were similar among the two strains. While splenic CD8 T cell number was similar among the two TRex strains, 1045^{+/+} MsIn^{-/-} mice exhibited increased splenic CD4 T cell frequency (FIG. 6A) and cell number. Over 95% of CD8 T cells expressed V39 (FIG. 6A) and were naive (CD44-CD62L+) (FIG. 6b) in both strains. ViSNE analysis (25), which reduces high-parameter data into 2 dimensions for visualization, confirmed a resting (CD25−Ki67−) T cell phenotype.

Peptide:MHC tetramer binding indicates T cell specificity and can be a proxy for both TCR affinity and functional avidity (2, 21-23). A fluorescently labeled MsIn406-414:H-2Db tetramer was generated to directly compare tetramer staining intensity between 7431 and 1045 T cells from TRex mice similar to as described (24). While 7431 and 1045 T cells expressed similar Vβ9, indicative of similar TCR cell surface levels, 1045 T cells stained brighter for tetramer (FIG. 6E). These data support that the 1045 TCR is higher affinity than the 7431 TCR, and consistent with prior results when these TCRs were expressed in P14 T cells (2). Effector T cells were next generated by in vitro stimulation of P14, 1045 and 7431 splenocytes with specific peptides (gp33 or MsIn406-414) and IL-2. On day 6 of expansion, the phenotypes of in vitro-derived effector T cells were compared by viSNE algorithm (25), which reduces high-parameter data into 2 dimensions for visualization. Expanded T cells upregulated CD44 yet maintained CD62L, consistent with antigen recognition and initial effector T cell differentiation. As anticipated, most expanded T cells were CD8+, and 1045 T cells were brighter for tetramer as compared to 7431 T cells. Notably, activated P14 T cells expressed higher PD-1 compared to activated T cells from TRex mice (FIG. 6F, 6J). PD1+P14 T cells were also particularly high for CD25 and CD69, molecules downstream of TCR signaling, suggesting a greater sustainment of TCR signaling as compared to T cells from TRex mice, even after just a single antigenic stimulation (FIG. 6F). 1045 and 7431 T cells with the highest CD25 and CD69 were also brightest for tetramer and Vβ9. Thus, directing physiological TCR expression, as in the 1045 and 7431 TRex mice, inhibits T cell over activation and potentially exhaustion by creating a more functionally diverse T cell pool. These data are consistent with prior human T cell engineering studies that showed targeting CARs or TCRs to the endogenous TRAC locus can promote physiological TCR signaling and reduce T cell exhaustion (26-29).

Effector T cell cytokine production was then measured in response to titrating antigen. Unexpectedly, 7431 effector T cells responded to a log lower peptide concentration compared to 1045 effector T cells (FIG. 6G). While 1045 effector T cells produced more IFNγ and TNFα in response to high peptide, 7431 effector T cells produced more of IFNγ and IL-2 in response to lower antigen on a per cell basis (FIG. 6H). Thus, despite decreased tetramer staining (FIG. 6E), 7431 effector T cells from TRex mice exhibit a higher functional avidity than 1045 T cells indicating that tetramer staining intensity is not always a surrogate for T cell avidity. Both 7431 and 1045 effector T cells were overall more responsive to antigen as compared to P14 T cells with regard to both the frequency of T cells producing cytokines and cytokines produced per cell (FIG. 6G-H), a result which could be due to how the TCR is regulated. 1045 effector T cells downregulated TCR to a greater extent than 7431 effector T cells particularly at lower antigen concentrations (FIG. 6I). In contrast, both 1045 and 7431 downregulated CD8 coreceptor similarly after antigen stimulation (FIG. 6I). Increased TCR downregulation at lower antigen levels by high affinity TCRs may be a compensatory mechanism to regulate cytokine production. Directing a CAR to the TRAC locus in human T cells promotes CAR internalization and re-expression which delays effector T-cell differentiation and acquisition of an exhausted phenotype (26). Targeting TCRs to TRAC also conferred productive antitumor human T cells (30).

An advantage of high affinity MHC I-restricted TCRs is their potential to engage CD4 helper T cells because they can bind peptide:MHC independent of the CD8 coreceptor (30). Therefore, MsIn tetramer binding was compared among the CD4+Vβ9+ T cells isolated from 1045 and 7431 TRex mice. While CD4 T cells isolated from 7431 and 1045 KI mice expressed similar TCR based on Vβ9 staining (FIG. 6K), CD4 T cells from 1045 mice stained significantly brighter for MsIn tetramer compared to CD4 T cells from 7431 mice (FIG. 6K). To interrogate functionality of the MHC I-restricted TCRs in CD4 T cells, splenocytes from 1045, 7431 and P14 mice were expanded in vitro for 6 days and then restimulated to measure cytokine production. A higher frequency of CD4+1045 T cells produced IFNγ and TNFα compared to 7431 and P14 T cells (FIG. 6I). Additionally, the amount of IFNγ produced per cell was significantly increased in 1045 CD4+ T cells (FIG. 6M-N). Thus, the 1045 TCR can elicit activation of CD4 T cells. Additionally, TCR downregulation was more pronounced in 1045 T cells at multiple timepoints (FIG. 6J). 1045 TRex T cells exhibited higher and prolonged CD25 and PD1 consistent with stronger TCR signaling (FIG. 6J).

### Bias Toward CD25-Tregs in MHC Class I TCR Transgenic Mice but not TRex Mice.

It was next assessed if Tregs were enriched in Trex mice based on observations that Tregs accumulate during αCD3+αCD28 and IL-2-induced expansion of P14 T cells (FIG. 2G-H). Ex vivo analysis showed that Tregs were disproportionally enriched among total CD4 T cells from P14 mice as compared to T cells from WT and TRex mice (FIG. 7A). Tregs were biased toward a CD25-Foxp3+ subset in P14 mice (FIG. 7A), which may be precursors to mature CD25+ Foxp3+ Treg (31, 32). To investigate the potential mechanism of Treg bias in P14 mice, proliferation was analyzed. A higher frequency of CD25-Foxp3+ Treg in P14 mice were Ki67+ compared to CD25− Tregs from TRex mice, yet this was not different from WT mice (FIG. 7B). A higher frequency of CD25-Ki67-Treg in P14 mice was noted compared to both WT and TRex Treg (FIG. 7B), suggesting either a survival advantage or bias toward generating this subset during T cell development. To test if Tregs were enriched in another TCR transgenic strain, Tregs in OT1 mice were quantified, which express a transgenic TCR specific to ovalbumin peptide in the context of H-2K^{b} (19). Similar to P14 mice, a significantly higher proportion of CD4 T cells were Treg in OT1 mice compared to WT mice (FIG. 7D). Further, most of the Tregs were biased toward a CD25-Foxp3+ phenotype (FIG. 7E). Thus, CD25− Treg subset accumulates in traditional TCR transgenic mice that does not occur in TRex mice.

While over 95% of Tregs in 1045 and 7431 TRex mice expressed the MsIn-specific MHC I restricted TCR, only ˜40% of Tregs expressed the transgenic TCR in P14 mice (FIG. 7C). However, all Tregs and conventional CD4 T cells expressed a functional TCR based on staining with pan anti-TCRβ in P14 mice, indicating that most Tregs are expressing endogenous TCRs in P14. In vitro expansion of splenocytes with peptide-pulsed APCs and IL-2 did not enrich for Tregs, but did increase the accumulation of activated conventional CD4 T cells. In vitro expansion of splenocytes from P14 mice with αCD3+αCD28 and IL-2 induced accumulation of Foxp3+ Tregs and this result was not observed in WT or Trex mice. These data are consistent with P14 T cells that were activated with αCD3+αCD28 and IL-2 prior to 7431 and 1045 TCR transduction (FIG. 2). Thus, in contrast to specific antigen recognition, αCD3+αCD28 and IL-2 can promote the expansion of Treg from splenocytes from TCR transgenic mice, but not lymphocytes from WT or Trex mice. These data may suggest Treg from TCR engineered animals are poised to respond differently to strong antigenic stimulation and IL-2 compared to WT Tregs.

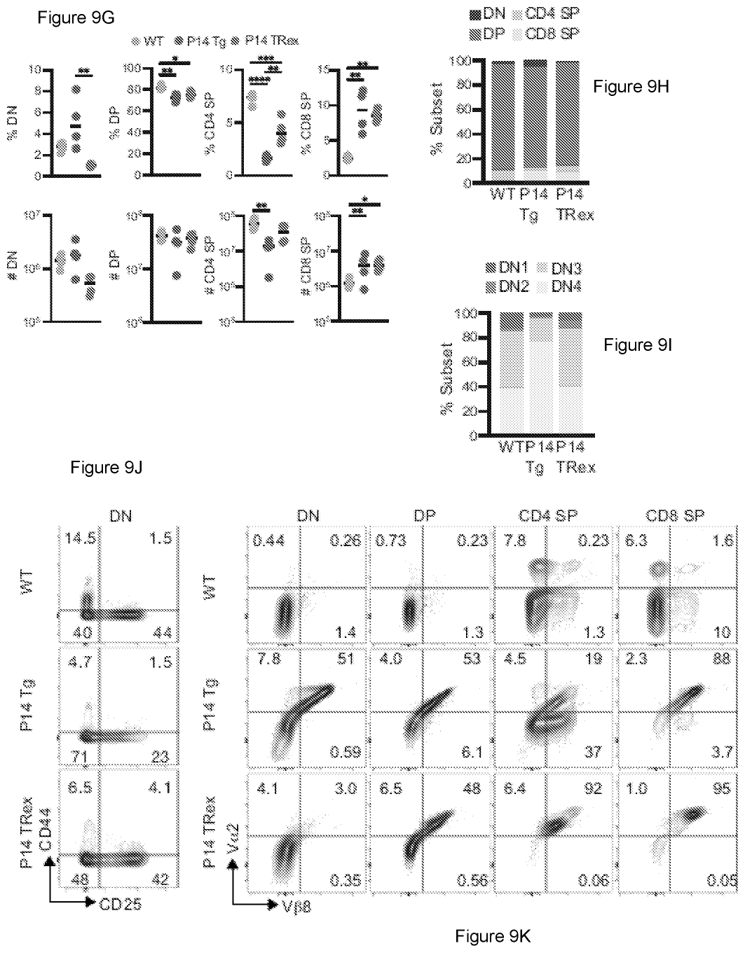

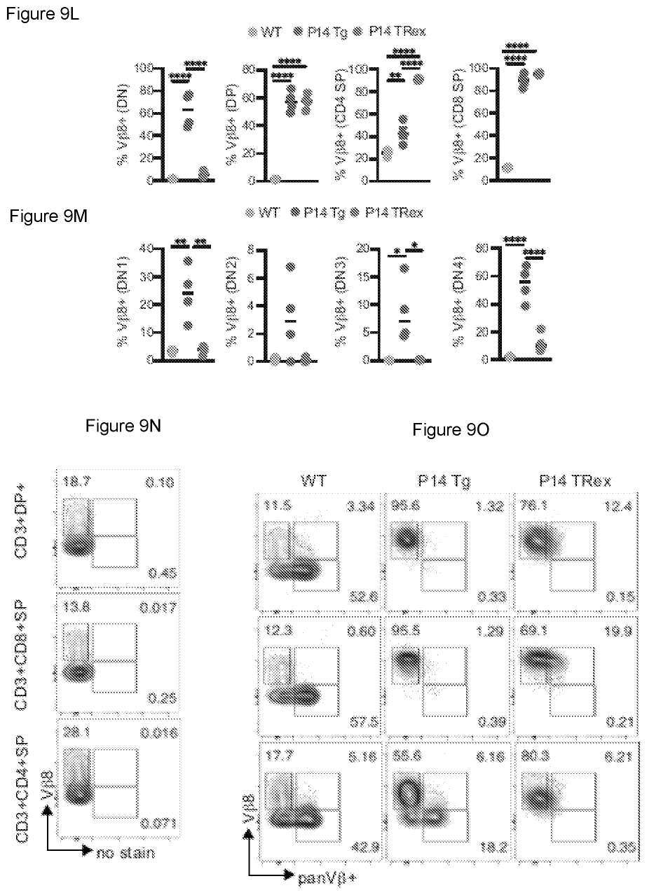

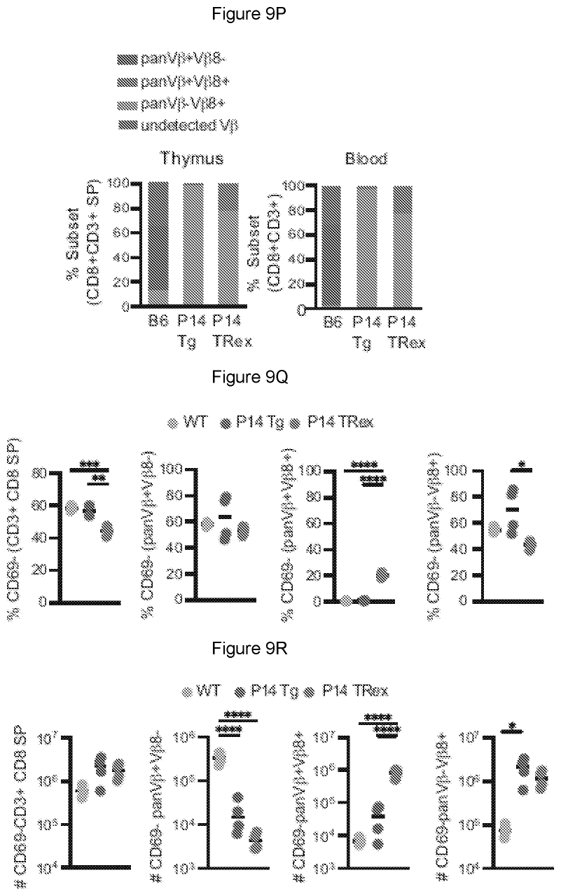

Generation and characterization of P14 TRex mice. To identify differences between the TRex approach and historical TCR transgenics, P14 TRex mice were generated. This approach was first tested in EL4 cells, which co-expressed P14 Vα2 and Vβ8 after gene editing (FIG. 9A). The P14 TCR was also integrated into Trac based on PCR (FIG. 9B). 19/52 pups (˜37%) integrated the P14 TCR in at least one Trac allele (FIG. 9B-C), a frequency within the range of 1045 and 7431 TRex pups (FIG. 9C). Circulating T cell proportions were biased toward the CD8 lineage and co-expressed Vα2 and Vβ8 in P14^{+/-} and P14^{+/+} pups (FIG. 9D). Thymus weight and immune cell number were similar among the strains (FIG. 9E). DN thymocyte frequency was increased in P14 Tg, and similarly low in P14 TRex and WT mice (FIG. 9F-H). DP and CD4 SP frequency was decreased and CD8 SP frequency was increased in both Tg and TRex mice compared to WT mice (FIG. 9F-H). CD4 SP number was decreased in P14 Tg compared to WT whereas CD8 SP number was similarly increased in both P14 strains (FIG. 9G). P14 Tg exhibited increased DN4 and decreased DN1-DN3 frequency as compared to P14 TRex and WT mice (FIG. 9I-J). While DN P14 Tg T cells were enriched for Vα2+Vβ8+ cells, TRex T cells became enriched for Vα2+Vβ8+ at later thymocyte development stages (FIG. 9K-L), consistent with the known timing of TCRα expression in WT thymocytes. Vβ8 was first detected at DN1 in P14 Tg thymocytes yet at the DN4 stage in TRex thymocytes (FIG. 9M). Only 20% of CD4 SP co-expressed Vα2 and Vβ8 in P14 Tg whereas over 90% of CD4 SP in TRex mice expressed the P14 TCR (FIG. 9K). Most CD8 SP in TRex mice were CD3+Vα2+Vβ8+(FIG. 9K-L). CD3e intensity was lower in CD4 SP than CD8 SP in P14 TRex mice and higher in CD8 SP in TRex than Tg and WT mice.

To assess endogenous Vβ in TRex mice, thymocytes were stained with a panel of antibodies specific various Vβ alleles. The Vβ panel detected 40-60% of endogenous Vβs in WT CD3+ thymocytes (FIG. 9N), an expected range since there are approximately 21 functional Vβ genes in mice (Khor et al., Current Opinion in Immunology 14: 230-234 (2002)). A fraction of CD3+ thymocytes lowly expressed an endogenous Vβ with high Vβ8 in TRex mice (FIG. 9N-0). P14 Tg and TRex DN4 thymocytes exhibited slightly increased dual Vβ frequencies compared to WT mice. While 20% of peripheral CD8, CD4 conventional (Tcon) and CD4+ Foxp3+ cells expressed both an endogenous and exogenous Vβ in P14 TRex mice (FIG. 9P), 40% of these T cell subsets were dual Vβ+ from the periphery of 1045^{+/+} MsIn^{-/-} TRex mice indicating some variability as to the extent endogenous Vβ is co-expressed. To investigate if thymocyte maturation is physiological in TRex mice, CD69 was quantified as it is downregulated in mature SP. CD69− CD8 SP expressed CD62L, another marker of mature SP. CD69− (mature) CD3+ CD8 SP thymocyte frequency was decreased in TRex mice as compared to P14 Tg and WT mice (FIG. 9Q). However, P14 Tg and TRex mice exhibited a 3-fold increase in mature CD3+ CD8 SP number as compared to WT mice (FIG. 9R), indicating CD8 SP maturation is intact in TRex mice.

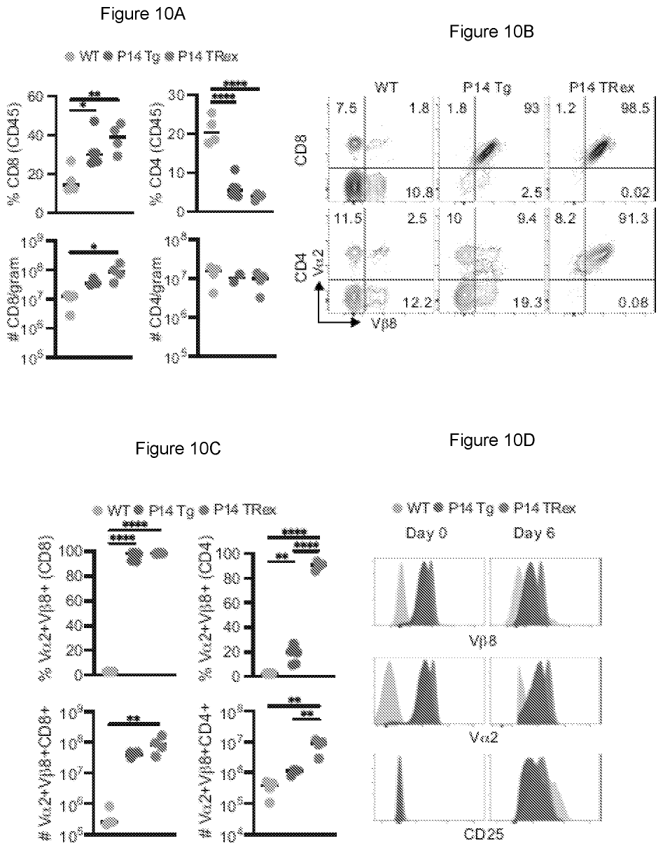

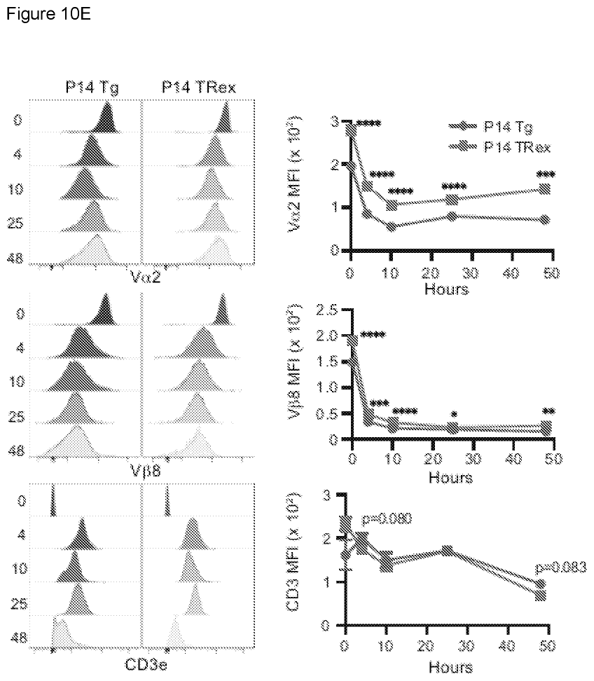

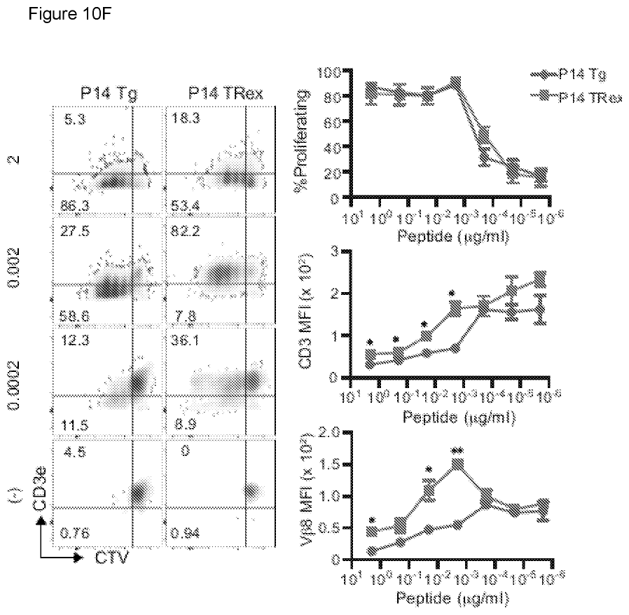

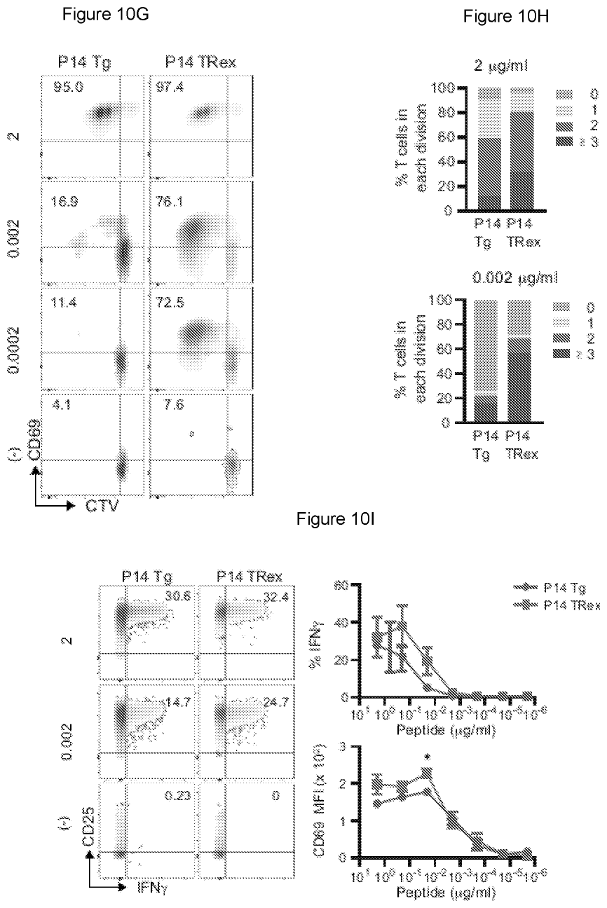

Targeting a TCR into Trac increases antigen sensitivity. Peripheral T cell responses were next compared between P14 TRex and Tg mice. Spleen weights were similar among the strains. CD8 T cell frequency and number were increased in both P14 strains as compared to WT mice (FIG. 10A). CD8 T cells equally co-expressed Vα2 and V38 among the two P14 strains (FIG. 10B), whereas more CD4 T cells were Vα2+Vβ8+ in TRex vs. Tg mice (FIG. 10B-C). Most CD8 T cells were naïve phenotype whereas most CD4 T cells expressed CD44 in both strains. TRex T cells expressed more CD3e, Vα2 and Vβ8 ex vivo (day 0) and following activation (day 6) than analogous P14 Tg T cells (FIG. 10D). CD25 was also higher in CD8 TRex than Tg effector T cells (FIG. 10D). However, the kinetics of TCR internalization and re-expression were similar in TRex and Tg T cells (FIG. 9E). Proliferation was then compared by incubating CTV-labeled splenocytes with titrating concentrations of antigen and IL-2. At low antigen concentration, TRex T cells were slightly more proliferative (FIG. 10F) and maintained higher TCR levels than Tg T cells (FIG. 10F). Providing exogenous IL-2 may compensate for differences in TCR signaling and proliferation in 9F and therefore we repeated the proliferation assay without IL-2. A greater frequency of TRex T cells were proliferating at low antigen concentration (FIG. 10G) corresponding to upregulation of CD69 (FIG. 10G) and CD44, whereas PD1 was not affected. Further, more TRex T cells had undergone ≥3 cell divisions (FIG. 10H) and were producing IFNγ than analogous Tg T cells (FIG. 10I). CD69 MFI and frequency of cells expressing CD25+ cells were greater in TRex vs. Tg effector T cells (FIG. 10I). Thus, the data suggest that the targeting a TCR to Trac can improve functional avidity and impact TCR internalization at low antigen densities.

### Discussion

It was shown previously in P14 mice most CD4 T cells do not express the transgenic TCR. However, CRISPR/Cas9 initiates a double-strand DNA break directly upstream of Trac resulting in a loss of endogenous TCRα and over 90% of both CD4 and CD8 T cells express the engineered TCR in TCR homozygous animals. Thus, donor rAAV-mediated TCR integration is critical for T cells to develop in TRex animals. This allows for a significant time advantage over historic methods of TCR engineering as further breeding to Rag^{-/-} or TCRα^{-/-} animals is no longer necessary. One limitation of the present study is that the endogenous TCRP chain remains intact in TRex mice, and thus the possibility that endogenous TCRP chains are co-expressed remains.

A previous approach to express MsIn-specific TCRs in murine T cells required γ-Retroviral vectors that co-expressed the desired TCRα and TCRP chains (2, 39, 40). There are numerous limitations with this previous approach. First, only 30-60% of T cells are transduced, necessitating further T cell stimulation and expansion to obtain sufficient numbers for cell therapy (2), a process that typically takes 2 weeks. Second, despite efforts to create optimized culture conditions to promote the fitness of activated murine T cells, as proven with human T cells (41), it is difficult to maintain murine T cell viability during repetitive in vitro stimulations with antigen. Thirdly, γ-Retroviral vectors can only transduce proliferating cells precluding the analysis of naïve MsIn-specific T cells. This is of interest because MsIn-expressing cancer vaccines are in clinical testing and target naïve MsIn-specific T cells (7, 42). Fourthly, retroviral vectors integrate randomly into the genome and can lead to insertional mutagenesis, oncogenesis, and experimental variability. In clinical trials, lentiviral-mediated chimeric antigen receptor (CAR) integration into TET2 or CBLB caused infused CAR T cell clonal expansion in cancer patients (43, 44). Additionally, gene silencing and variable non-uniform receptor expression can occur following retroviral transduction of T cells (26, 45, 46).

TCR transgenic mice have improved the understanding of T cell development and differentiation. There are some limitations to this approach including TCRs are randomly integrated into the genome, often in multiple locations, and TCR expression and regulation is dependent often on non-physiologic heterologous promoter fragments. TCR rearrangement is a highly ordered and sequential process where TCRβ is rearranged in DN3 preceding TCRα rearrangement at later DN4 and DP stages. A productive TCRβ rearrangement prevents further Vα-to-DJβ rearrangements at the DP stage, a process called allelic exclusion (Khor et al., Current Opinion in Immunology 14 (2002)). Premature TCRα and TCRβ expression at the DN1 stage in historical TCR transgenics can impact thymocyte development (38). In TRex mice, it was shown that TCRα and TCRβ are first expressed in DN4, the timing of endogenous TCRα expression and TRex thymocytes undergo all the sequential stages of thymocyte maturation. It was identified that MHC I is required for positive selection of TRex T cells and self/tumor-reactive high affinity thymocytes undergo negative selection in an antigen-dependent manner.

One consideration of the TRex approach is a fraction of TRex T cells express endogenous TCRβ in addition to the exogenous TCRβ. However, it was identified herein that fewer TRex T cells express endogenous TCRβ than WT T cells and endogenous TCRβ cell surface expression is much lower in TRex T cells vs. WT T cells. It was also shown that more CD4 T cells express the P14 TCR in TRex mice vs. transgenic mice, which is consistent with multiple endogenous TCRα in P14 transgenic T cells. Thus, the data herein suggest that lack of allelic exclusion at the alpha locus permits more TCR pairings, whereas lack of allelic exclusion at the beta locus is not as permissive to alternative TCR pairings potentially because mechanisms are in play to silence an endogenous TCRβ. Thus, post-transcriptional mechanisms for silencing endogenous TCRβ (Steinel et al., J. Immunol. 185, (2010); Levin-Klein et al., Frontiers in Immunology 5 (2014)) may be playing a role in TRex T cells thereby forcing exogenous TCR expression for successful CD4 and CD8 T lymphocyte maturation. Building on the TRex model, endogenous TCRβ could be deleted using CRISPR/Cas9 but exogenous TCRβ must remain intact. Alternatively, TRex mice could be generated directly onto a TCRβ^{-/-} background, potentially saving time over historical TCR transgenic mice that are often bred to a Rag^{-/-} or TCRα^{-/-} background to ensure that only the transgenic TCR is expressed (38). As CRISPR/Cas9 initiates a double-strand DNA break directly upstream of Trac resulting in a loss of endogenous TCRα, exogenous TCR integration is critical for T cell development in TRex animals.

Particular to TCR engineering, exogenous TCRs must compete with endogenous TCRs for CD3 complex and cell surface expression resulting in reduced exogenous TCR expression and decreased T cell avidity and cancer cell recognition (47). Due to the lack of competition with endogenous TCRs, human T cells lentivirally transduced to express a TCR combined with knocking out TCRP were up to a thousand-fold more sensitive to antigen than standard TCR-transduced T cells (27). Exogenous TCRα and TCRP chains can also mispair with endogenous TCR chains, resulting in unknown T cell antigen specificities and increasing potential for cross-reactivity to normal tissues (40). Due to the challenges associated with outcompeting endogenous polyclonal TCRs, P14 TCR transgenic T cells (10) were previously used as the murine T cell source for engineering because exogenous TCRs outcompete the P14 TCR but fail to outcompete polyclonal TCRs. However, in P14 mice, T cells are largely biased toward the CD8 T cell lineage with few CD4 T cells. As engineered CD4+ T cells contribute to CAR T cell anti-tumor activity (48), the prior approach was limited to assessing only TCR engineered CD8 T cells. Here, it was found that the high affinity 1045 TCR functions in CD4 T cells from Trex mice permitting future studies to potentiate the antitumor function of MHC I-restricted TCR engineered CD4 T cells.

It was set out to address the above hurdles by creating both high affinity (1045) and low affinity (7431) TRex animals. Most (>95%) CD4 and CD8 T cells express the engineered TCR within the physiologic locus and these T cells are highly responsive to specific antigen. Generation of the 1045 TRex mice in which CD4 T cells are functional permit novel studies to potentiate the antitumor function of MHC I-restricted CD4 T cells. While the studies herein support that 1045 T cells are not tolerized in MsIn^{+/+} animals, additional investigation beyond the scope of the current study will be necessary to fully analyze the role of MsIn in the development of T cells from TRex mice. Unexpectedly, despite lower tetramer binding and a presumably lower affinity TCR, 7431 T cells are more functional than 1045 T cells when antigen is limiting. These data contrast with a prior study that showed 1045-retrovirally transduced T cells exhibited greater sensitivity to lower antigen concentration as compared to 7431-retrovirally transduced T cells (2). Based on greater TCR downregulation in 1045 T cells vs. 7431 T cells following antigen recognition, it is possible that stronger TCR signaling compensates by TCR downregulation. Prior studies of other T cell specificities support that tetramer staining is not always a surrogate for T cell functionality (49, 50).

T cells that express high affinity self-reactive TCRs are susceptible to thymic negative selection, an essential central tolerance mechanism that safeguards against autoimmunity. Here, it was identified that both copies of MsIn are necessary for negative selection of high affinity MsIn-specific T cells supporting a gene dosage dependent mechanism of central tolerance. Loss of one MsIn allele may reduce protein expression on a per cell basis. Alternatively, as MsIn is expression has been reported in mTECs, may be Aire-dependent (57) and Aire-dependent genes can be stochastically monoallelically expressed (58), MsIn allele loss may reduce the number of MsIn+ thymic APCs that mediate negative selection. Fezf2 elicits self-antigen expression in mTECs in an Aire-independent manner (59) and also represses some mTEC genes including MsIn (60) suggesting MsIn may not be particularly highly expressed by mTECs and are consistent with our results that both MsIn alleles are required for negative selection to this antigen. MSLN is detected in Hassall's corpuscles in the normal human thymus (Inaguma et al. Oncotarget 8:26744-26754, 2017) and single cell sequencing show MSLN in both thymic mesothelial cells and epithelial cells (61). MSLN is also overexpressed in thymic carcinomas (62). Thus, further investigation into the thymic cell type(s) that induce negative selection of MsIn-specific T cells is warranted.

The present study supports that the genomic location of TCR can impact T cell effector functionality. It was found find that both transgenic T cells and T cells retrovirally-transduced with MsIn-specific TCRs are less functional based on cytokine production than T cells in which the MsIn-specific TCR is in the Trac locus. Despite a lower affinity TCR, 7431 TRex effector T cells were as sensitive to low antigen as 1045 TRex effector T cells. While tetramer staining is not always a surrogate for T cell functionality (63, 64), 1045 RV T cells exhibited a higher functional avidity as compared to 7431 RV T cells (2). P14 TRex T cells were also more sensitive to antigen as compared to P14 transgenic T cells and this may be explained in part due to higher TCR in TRex T cells. Thus, Trac targeting may improve antigen sensitivity of lower affinity TCRs. The sustained and elevated PD-1 and T cell activation markers CD25 and CD69 in P14 T cells, even after a single antigenic stimulation, was striking and distinct from results obtained from activated T cells from TRex mice. Further, primary murine T cells with the MsIn-specific TCRs contained within Trac exhibited enhanced T cell function over multiple stimulations in vitro compared to T cells with the identical TCRs retrovirally expressed in P14 T cells. Human T cells engineered with a CAR expressed in the TRAC locus had superior antitumor activity compared to T cells that had undergone random lentiviral-mediated CAR integration in a xenogeneic leukemia model (26). T cells with TRAC-integrated CARs were resistant to exhaustion because the CAR was physiologically down-regulated during chronic antigen exposure (26). The present results in murine T cells are supported by human T cell studies that replaced endogenous TCRs with exogenous TCRs which led to specific antigen recognition, cytokine release and tumor cell killing (28) and physiological TCR signaling (29).

Unexpectedly, it was identified that Foxp3+ Tregs are enriched among total CD4 T cells in traditional MHC class I-restricted TCR transgenic animals but not in TRex mice. It was also shown that Foxp3+ Tregs accumulate during αCD3+αCD28 and IL-2 in vitro stimulation of P14 or TRex T cells, but not in WT mice. These Tregs may differentiate from conventional helper T cells and/or expand during strong TCR and costimulatory signals and IL-2. Further investigation into this mechanism could influence how T cells are cultured for adoptive cell therapy, as Treg expansion is likely a limitation of the prior TCR engineering approach (2,11). While the mechanism and consequence of Treg bias will require further investigation, the strategy here, which allows for peptide to robustly induce the expansion of CD8 T cells from TRex mice obviates this issue for preclinical studies. In sum, a highly efficient and reproducible approach to develop novel physiologically-regulated antigen-specific TRex models has been designed. It is expected that the MsIn TRex mice generated here will prove useful to inform the design of safe effective immunotherapies for solid tumor eradication, and antigen specific TCRs designed in this manner will contribute to many therapeutic areas. Thus, the TRex method is an efficient modality to generate TCR expressing strains and the TRex models herein may be useful to elucidate the role of physiological TCR regulation on T cell function in diverse biological contexts.

It is understood that every embodiment of the disclosure described herein may optionally be combined with any one or more of the other embodiments described herein. Every patent literature and every non-patent literature cited herein are incorporated herein by reference in their entirety.

It is understood, therefore, that this invention is not limited to the particular embodiments disclosed, but is intended to cover all modifications which are within the spirit and scope of the invention as defined by the appended claims; the above description, and/or shown in the attached drawings. Consequently only such limitations as appear in the appended claims should be placed on the disclosure.

### REFERENCES

1. Argani, P., C. Iacobuzio-Donahue, C. Rosty, M. Goggins, R. E. Wilentz, S. R. Murugesan, E. Jaffee, S. E. Kern, R. H. Hruban, B. Ryu, S. D. Leach, C. J. Yeo, and J. L. Cameron. 2001. Mesothelin is overexpressed in the vast majority of ductal adenocarcinomas of the pancreas: Identification of a new pancreatic cancer marker by serial analysis of gene expression (SAGE). Clin. Cancer Res.
2. Stromnes, I. M., T. M. Schmitt, A. Hulbert, J. S. Brockenbrough, H. N. Nguyen, C. Cuevas, A. M. Dotson, X. Tan, J. L. Hotes, P. D. Greenberg, and S. R. Hingorani. 2015. T Cells Engineered against a Native Antigen Can Surmount Immunologic and Physical Barriers to Treat Pancreatic Ductal Adenocarcinoma. Cancer Cell 28.
3. Hassan, R., A. Thomas, C. Alewine, D. T. Le, E. M. Jaffee, and I. Pastan. 2016. Mesothelin immunotherapy for cancer: Ready for prime time?J. Clin. Oncol.
4. Coelho, R., S. Ricardo, A. L. Amaral, Y. L. Huang, M. Nunes, J. P. Neves, N. Mendes, M. N. López, C. Bartosch, V. Ferreira, R. Portugal, J. M. Lopes, R. Almeida, V. Heinzelmann-Schwarz, F. Jacob, and L. David. 2020. Regulation of invasion and peritoneal dissemination of ovarian cancer by mesothelin manipulation. Oncogenesis.
5. Thomas, A., Y. Chen, S. M. Steinberg, J. Luo, S. Pack, M. Raffeld, Z. Abdullaev, C. Alewine, A. Rajan, G. Giaccone, I. Pastan, M. Miettinen, and R. Hassan. 2015. High mesothelin expression in advanced lung adenocarcinoma is associated with KRAS mutations and a poor prognosis. Oncotarget.
6. Tchou, J., L. C. Wang, B. Selven, H. Zhang, J. Conejo-Garcia, H. Borghaei, M. Kalos, R. H. Vondeheide, S. M. Albelda, C. H. June, and P. J. Zhang. 2012. Mesothelin, a novel immunotherapy target for triple negative breast cancer. Breast Cancer Res. Treat.
7. Thomas, A. M., L. M. Santarsiero, E. R. Lutz, T. D. Armstrong, Y. C. Chen, L. Q. Huang, D. A. Laheru, M. Goggins, R. H. Hruban, and E. M. Jaffee. 2004. Mesothelin-specific CD8+ T cell responses provide evidence of in vivo cross-priming by antigen-presenting cells in vaccinated pancreatic cancer patients. J. Exp. Med.
8. Bera, T. K., and I. Pastan. 2000. Mesothelin Is Not Required for Normal Mouse Development or Reproduction. Mol. Cell. Biol. 20.
9. Pastan, I., and R. Hassan. 2014. Discovery of mesothelin and exploiting it as a target for immunotherapy. Cancer Res.
10. Pircher, H., K. BQrki, R. Lang, H. Hengartner, and R. M. Zinkernagel. 1989. Tolerance induction in double specific T-cell receptor transgenic mice varies with antigen. Nature (1989).
11. Anderson, K. G., V. Voillet, B. M. Bates, E. Y. Chiu, M. G. Burnett, N. M. Garcia, S. K. Oda, C. B. Morse, I. M. Stromnes, C. W. Drescher, R. Gottardo, and P. D. Greenberg. 2019. Engineered adoptive t-cell therapy prolongs survival in a preclinical model of advanced-stage ovarian cancer. Cancer Immunol. Res. 7.
12. Stromnes, I. M., A. L. Burrack, A. Hulbert, P. Bonson, C. Black, J. S. Brockenbrough, J. F. Raynor, E. J. Spartz, R. H. Pierce, P. D. Greenberg, and S. R. Hingorani. 2019. Differential effects of depleting versus programming tumor-associated macrophages on engineered T cells in pancreatic ductal adenocarcinoma. Cancer Immunol. Res. 7.
13. Sant'Angelo, D. B., G. Waterbury, P. Preston-Hurlburt, S. T. Yoon, R. Medzhitov, S. C. Hong, and C. A. Janeway. 1996. The specificity and orientation of a TCR to its peptide-MHC class II ligands. Immunity 4.
14. Wang, J., J. J. DeClercq, S. B. Hayward, P. W. L. Li, D. A. Shivak, P. D. Gregory, G. Lee, and M. C. Holmes. 2016. Highly efficient homology-driven genome editing in human T cells by combining zinc-finger nuclease mRNA and AAV6 donor delivery. Nucleic Acids Res. 44.
15. Liu, Z., O. Chen, J. B. J. Wall, M. Zheng, Y. Zhou, L. Wang, H. Ruth Vaseghi, L. Qian, and J. Liu. 2017. Systematic comparison of 2A peptides for cloning multi-genes in a polycistronic vector. Sci. Rep. 7.
16. Seki, A., and S. Rutz. 2018. Optimized RNP transfection for highly efficient CRI SPR/Cas9-mediated gene knockout in primary T cells. J. Exp. Med.
17. Starr, T. K., S. C. Jameson, and K. A. Hogquist. 2003. Positive and negative selection of T cells. Annu. Rev. Immunol.
18. Chen, S., S. Sun, D. Moonen, C. Lee, A. Y. F. Lee, D. V. Schaffer, and L. He. 2019. CRISPR-READI: Efficient Generation of Knockin Mice by CRISPR RNP Electroporation and AAV Donor Infection. Cell Rep.
19. Hogquist, K. A., S. C. Jameson, W. R. Heath, J. L. Howard, M. J. Bevan, and F. R. Carbone. 1994. T cell receptor antagonist peptides induce positive selection. Cell 76.
20. Wooldridge, L., H. A. Van Den Berg, M. Glick, E. Gostick, B. Laugel, S. L. Hutchinson, A. Milicic, J. M. Brenchley, D. C. Douek, D. A. Price, and A. K. Sewell. 2005. Interaction between the CD8 coreceptor and major histocompatibility complex class I stabilizes T cell receptor-antigen complexes at the cell surface. J. Biol. Chem. 280.
21. Crawford, F., H. Kozono, J. White, P. Marrack, and J. Kappler. 1998. Detection of antigen-specific T cells with multivalent soluble class II MHC covalent peptide complexes. Immunity 8.
22. Yee, C., P. A. Savage, P. P. Lee, M. M. Davis, and P. D. Greenberg. 1999. Isolation of high avidity melanoma-reactive CTL from heterogeneous populations using peptide-MHC tetramers. J. Immunol. 162.
23. Stone, J. D., A. S. Chervin, and D. M. Kranz. 2009. T-cell receptor binding affinities and kinetics: impact on T-cell activity and specificity. Immunology 126.
24. Burrack, A. L. A. L., E. J. E. J. Spartz, J. F. J. F. Raynor, I. Wang, M. Olson, and I. M. I. M. Stromnes. 2019. Combination PD-1 and PD-L1 Blockade Promotes Durable Neoantigen-Specific T Cell-Mediated Immunity in Pancreatic Ductal Adenocarcinoma. Cell Rep. 28.
25. Amir, E. A. D., K. L. Davis, M. D. Tadmor, E. F. Simonds, J. H. Levine, S. C. Bendall, D. K. Shenfeld, S. Krishnaswamy, G. P. Nolan, and D. Pe'Er. 2013. ViSNE enables visualization of high dimensional single-cell data and reveals phenotypic heterogeneity of leukemia. Nat. Biotechnol. 31.
26. Eyquem, J., J. Mansilla-Soto, T. Giavridis, S. J. C. Van Der Stegen, M. Hamieh, K. M. Cunanan, A. Odak, M. Gónen, and M. Sadelain. 2017. Targeting a CAR to the TRAC locus with CRISPR/Cas9 enhances tumour rejection. Nature
27. Legut, M., G. Dolton, A. A. Mian, O. G. Ottmann, and A. K. Sewell. 2018. CRISPR-mediated TCR replacement generates superior anticancer transgenic t cells. Blood
28. Roth, T. L., C. Puig-Saus, R. Yu, E. Shifrut, J. Carnevale, P. J. Li, J. Hiatt, J. Saco, P. Krystofinski, H. Li, V. Tobin, D. N. Nguyen, M. R. Lee, A. L. Putnam, A. L. Ferris, J. W. Chen, J. N. Schickel, L. Pellerin, D. Carmody, G. Alkorta-Aranburu, D. Del Gaudio, H. Matsumoto, M. Morell, Y. Mao, M. Cho, R. M. Quadros, C. B. Gurumurthy, B. Smith, M. Haugwitz, S. H. Hughes, J. S. Weissman, K. Schumann, J. H. Esensten, A. P. May, A. Ashworth, G. M. Kupfer, S. A. W. Greeley, R. Bacchetta, E. Meffre, M. G. Roncarolo, N. Romberg, K. C. Herold, A. Ribas, M. D. Leonetti, and A. Marson. 2018. Reprogramming human T cell function and specificity with non-viral genome targeting. Nature 559.
29. Schober, K., T. R. Müller, F. Gökmen, S. Grassmann, M. Effenberger, M. Poltorak, C. Stemberger, K. Schumann, T. L. Roth, A. Marson, and D. H. Busch. 2019. Orthotopic replacement of T-cell receptor α- and β-chains with preservation of near-physiological T-cell function. Nat. Biomed. Eng. 3.
30. Soto, C. M., J. D. Stone, A. S. Chervin, B. Engels, H. Schreiber, E. J. Roy, and D. M. Kranz. 2013. MHC-class I-restricted CD4 T cells: A nanomolar affinity TCR has improved anti-tumor efficacy in vivo compared to the micromolar wild-type TCR. Cancer Immunol. Immunother. 62.
31. Tai, X., B. Erman, A. Alag, J. Mu, M. Kimura, G. Katz, T. Guinter, T. McCaughtry, R. Etzensperger, L. Feigenbaum, D. S. Singer, and A. Singer. 2013. Foxp3 Transcription Factor Is Proapoptotic and Lethal to Developing Regulatory T Cells unless Counterbalanced by Cytokine Survival Signals. Immunity 38.
32. Schuster, M., C. Plaza-Sirvent, A. Visekruna, J. Huehn, and I. Schmitz. 2019. Generation of Foxp3+ CD25− regulatory T-cell precursors requires c-rel and IκBNS. Front. Immunol. 10.
33. Mamalaki, C., J. Elliott, T. Norton, N. Yannoutsos, D. Kioussis, A. R. Townsend, P. Chandler, and E. Simpson. 1993. Positive and Negative Selection in Transgenic Mice Expressing a T-Cell Receptor Specific for Influenza Nucleoprotein and Endogenous Superantigen. Dev. Immunol.
34. Xue, S. A., G. M. Bendle, A. Holler, and H. J. Stauss. 2008. Generation and characterization of transgenic mice expressing a T-cell receptor specific for the tumour-associated antigen MDM2. Immunology.
35. Kouskoff, V., K. Signorelli, C. Benoist, and D. Mathis. 1995. Cassette vectors directing expression of T cell receptor genes in transgenic mice. J. Immunol. Methods
36. Katz, J. D., B. Wang, K. Haskins, C. Benoist, and D. Mathis. 1993. Following a diabetogenic T cell from genesis through pathogenesis. Cell
37. Uematsu, Y., S. Ryser, Z. Dembid, P. Borgulya, P. Krimpenfort, A. Berns, H. von Boehmer, and M. Steinmetz. 1988. In transgenic mice the introduced functional T cell receptor β gene prevents expression of endogenous p genes. Cell
38. Baldwin, T. A., M. M. Sandau, S. C. Jameson, and K. A. Hogquist. 2005. The timing of TCRα expression critically influences T cell development and selection. J. Exp. Med. 202.
39. Stromnes, I. M., T. M. Schmitt, A. G. Chapuis, S. R. Hingorani, and P. D. Greenberg. 2014. Re-adapting T cells for cancer therapy: From mouse models to clinical trials. Immunol. Rev. 257.
40. Stromnes, I. M. I. M., T. M. T. M. Schmitt, A. G. A. G. Chapuis, S. R. S. R. Hingorani, and P. D. P. D. Greenberg. 2014. Re-adapting T cells for cancer therapy: From mouse models to clinical trials. Immunol. Rev. 257.
41. Rollins, M. R. M. R., E. J. E. J. Spartz, and I. M. I. M. Stromnes. 2020. T Cell Receptor Engineered Lymphocytes for Cancer Therapy. Curr. Protoc. Immunol. 129.
42. Le, D. T., A. Wang-Gillam, V. Picozzi, T. F. Greten, T. Crocenzi, G. Springett, M. Morse, H. Zeh, D. Cohen, R. L. Fine, B. Onners, J. N. Uram, D. A. Laheru, E. R. Lutz, S. Solt, A. L. Murphy, J. Skoble, E. Lemmens, J. Grous, T. Dubensky, D. G. Brockstedt, and E. M. Jaffee. 2015. Safety and survival with GVAX pancreas prime and Listeria monocytogenes-expressing mesothelin (CRS-207) boost vaccines for metastatic pancreatic cancer. J. Clin. Oncol.
43. Fraietta, J. A., C. L. Nobles, M. A. Sammons, S. Lundh, S. A. Carty, T. J. Reich, A. P. Cogdill, J. J. D. Morrissette, J. E. DeNizio, S. Reddy, Y. Hwang, M. Gohil, I. Kulikovskaya, F. Nazimuddin, M. Gupta, F. Chen, J. K. Everett, K. A. Alexander, E. Lin-Shiao, M. H. Gee, X. Liu, R. M. Young, D. Ambrose, Y. Wang, J. Xu, M. S. Jordan, K. T. Marcucci, B. L. Levine, K. C. Garcia, Y. Zhao, M. Kalos, D. L. Porter, R. M. Kohli, S. F. Lacey, S. L. Berger, F. D. Bushman, C. H. June, and J. J. Melenhorst. 2018. Disruption of TET2 promotes the therapeutic efficacy of CD19-targeted T cells. Nature
44. Shah, N. N., H. Qin, B. Yates, L. Su, H. Shalabi, M. Raffeld, M. A. Ahlman, M. Stetler-Stevenson, C. Yuan, S. Guo, S. Liu, S. H. Hughes, T. J. Fry, and X. Wu. 2019. Clonal expansion of CAR T cells harboring lentivector integration in the CBL gene following anti-CD22 CAR T-cell therapy. Blood Adv.
45. Von Kalle, C., A. Deichmann, and M. Schmidt. 2014. Vector integration and tumorigenesis. Hum. Gene Ther.
46. Ellis, J. 2005. Silencing and variegation of gammaretrovirus and lentivirus vectors. Hum. Gene Ther.
47. Ahmadi, M., J. W. King, S. A. Xue, C. Voisine, A. Holler, G. P. Wright, J. Waxman, E. Morris, and H. J. Stauss. 2011. CD3 limits the efficacy of TCR gene therapy in vivo. Blood
48. Turtle, C. J., L. A. Hanafi, C. Berger, T. A. Gooley, S. Cherian, M. Hudecek, D. Sommermeyer, K. Melville, B. Pender, T. M. Budiarto, E. Robinson, N. N. Steevens, C. Chaney, L. Soma, X. Chen, C. Yeung, B. Wood, D. Li, J. Cao, S. Heimfeld, M. C. Jensen, S. R. Riddell, and D. G. Maloney. 2016. CD19 CAR-T cells of defined CD4+:CD8+ composition in adult B cell ALL patients. J. Clin. Invest.
49. Rius, C., M. Attaf, K. Tungatt, V. Bianchi, M. Legut, A. Bovay, M. Donia, P. thor Straten, M. Peakman, I. M. Svane, S. Ott, T. Connor, B. Szomolay, G. Dolton, and A. K. Sewell. 2018. Peptide-MHC Class I Tetramers Can Fail To Detect Relevant Functional T Cell Clonotypes and Underestimate Antigen-Reactive T Cell Populations. J. Immunol. 200.
50. Tungatt, K., V. Bianchi, M. D. Crowther, W. E. Powell, A. J. Schauenburg, A. Trimby, M. Donia, J. J. Miles, C. J. Holland, D. K. Cole, A. J. Godkin, M. Peakman, P. T. Straten, I. M. Svane, A. K. Sewell, and G. Dolton. 2015. Antibody Stabilization of Peptide-MHC Multimers Reveals Functional T Cells Bearing Extremely Low-Affinity TCRs. J. Immunol. 194.
51. Yang, S., C. J. Cohen, P. D. Peng, Y. Zhao, L. Cassard, Z. Yu, Z. Zheng, S. Jones, N. P. Restifo, S. A. Rosenberg, and R. A. Morgan. 2008. Development of optimal bicistronic lentiviral vectors facilitates high-level TCR gene expression and robust tumor cell recognition. Gene Ther. 15.
52. Gibson, D. G. 2011. Enzymatic assembly of overlapping DNA fragments. In Methods in Enzymology vol. 498.
53. Behringer, R., M. Gertsenstein, K. Vintersen Nagy, and A. Nagy. 2014. Manipulating the Mouse Embryo.
54. Kito, S., T. Hayao, Y. Noguchi-Kawasaki, Y. Ohta, H. Uhara, and S. Tateno. 2004. Improved in vitro fertilization and development by use of modified human tubal fluid and applicability of pronucleate embryos for cryopreservation by rapid freezing in inbred mice. Comp. Med.
55. Xianghong Li et al. Cas-CLOVER™: A High-Fidelity Genome Editing System for Safe and Efficient Modification of Cells for Immunotherapy. 2018 Precision CR/SPR Congress Poster Presentation, Boston, MA
56. Class 2 CRISPR/Cas: an expanding biotechnology toolbox for and beyond genome editing Yuyi Tang & Yan Fu Cell & Bioscience volume 8, Article number: 59 (2018)
57. Derbinski, J. et al. Promiscuous gene expression in thymic epithelial cells is regulated at multiple levels. J. Exp. Med. 202, (2005).
58. Villasenor, J., Besse, W., Benoist, C. & Mathis, D. Ectopic expression of peripheral-tissue antigens in the thymic epithelium: Probabilistic, monoallelic, misinitiated. Proc. Natl. Acad. Sci. U.S.A 105, (2008).
59. Takaba, H. et al. Fezf2 Orchestrates a Thymic Program of Self-Antigen Expression for Immune Tolerance. Cell 163, (2015).
60. Tomofuji, Y. et al. Chd4 choreographs self-antigen expression for central immune tolerance. Nat. Immunol. 21, (2020).
61. Bautista, J. L. et al. Single-cell transcriptional profiling of human thymic stroma uncovers novel cellular heterogeneity in the thymic medulla. Nat. Commun. 12, (2021).
62. Chen, Y. et al. Mesothelin expression in thymic epithelial tumors (TETs). J. Clin. Oncol. 32, (2014).
63. Rius, C. et al. Peptide-MHC Class I Tetramers Can Fail To Detect Relevant Functional T Cell Clonotypes and Underestimate Antigen-Reactive T Cell Populations. J. Immunol. 200, (2018).
64. Tungatt, K. et al. Antibody Stabilization of Peptide-MHC Multimers Reveals Functional T Cells Bearing Extremely Low-Affinity TCRs. J. Immunol. 194, (2015).

What is claimed:

1. A genetically engineered non-human animal comprising
i) a TCR exchanged (TRex) T cell receptor locus expressing a T cell receptor specific for mesothelin, wherein the non-human animal also expresses less than 15% endogenous T cell receptor on TCR α or β expressing cells; and
ii) an inactivated mesothelin gene.

2. The genetically engineered non-human animal of claim 1 wherein the TCR exchange is introduced in the T cell receptor alpha (Trac) locus.

3. The genetically engineered non-human animal of claim 1 or 2, wherein the TCR exchange comprises nuclease-dependent cleavage system disruption of the Trac locus and introduction of a polynucleotide encoding the T cell receptor specific for mesothelin.

4. The genetically engineered animal of claim 3, wherein the nuclease dependent cleavage system comprises a CRISPR/Cas system, a Cas-CLOVER system, a zinc-finger nuclease (ZFN) system, a transcription activator like effector nuclease (TALEN) system, or a meganuclease system.

5. The genetically engineered animal of claim 4, wherein the CRISPR/Cas system comprises Cas9, Cas12a, Cas13a or Cas13b.

6. The genetically engineered non-human animal of any one of claims 1-5, wherein the polynucleotide encoding the T cell receptor specific for mesothelin is expressed on a viral vector, optionally an AAV vector.

7. The genetically engineered non-human animal of any one of claims 1 to 6, wherein the animal expresses high affinity mesothelin-specific T cells.

8. The genetically engineered non-human animal of any one of claims 1 to 6, wherein the animal expresses low affinity mesothelin-specific T cells.

9. The genetically engineered non-human animal of any one of claims 1 to 8, wherein the T cells expressing the mesothelin-specific TCR are CD4+ T cells or CD8+ T cells.

10. The genetically engineered non-human animal of any one of claims 1 to 7, wherein the animal is a mouse.

11. The genetically engineered non-human animal of claim 10, wherein high affinity mesothelin-specific T cells express a 1045 TCR having the amino acid sequence set out in FIG. 7.

12. The genetically engineered non-human animal of claim 10, wherein low affinity mesothelin-specific T cells express a 7431 TCR having the amino acid sequence set out in FIG. 7.

13. The genetically engineered non-human animal of claim 10 to 12, wherein the mouse is on a C57Bl/6 background or NOD background.

14. The genetically engineered non-human animal of any one of claims 1 to 13, wherein the mesothelin gene is disrupted in exon 4 of the mesothelin gene.

15. The genetically engineered non-human animal of any one of claims 1 to 14, wherein the animal is homozygous for the donor TCR or heterozygous for the donor TCR.

16. The genetically engineered non-human animal of any one of claims 1 to 15 wherein the animal is homozygous for the mesothelin knockout.

17. A T cell expressing a T cell receptor specific for mesothelin isolated from a genetically engineered non-human animal of any one of claims 1 to 16.

18. The T cell of claim 17 which is a CD4+ T cell or CD8+ T cell.

19. The T cell of claim 17 or 18, wherein the T cell is an effector T cell or a memory T cell.

20. The T cell of any one of claims 17 to 19, wherein the T cell is CD44^{low}/CD62L+CD44highCD26L− or CD44highCD62L+.

21. A method of measuring effects of T cells having TCR exchanged (TRex) T cell receptor locus expressing a T cell receptor specific for mesothelin comprising contacting a T cell of claim 17 to 20 with mesothelin presented in MHC and measuring the effects on the T cell.

22. The method of claim 21, wherein the effects include stimulation of cytokine production, modulation of cell surface marker phenotype, change in activation phenotype, modulation of number of regulatory T cells induced, or cytotoxicity phenotype, replicating endogenous TCR gene regulation following antigen encounter, and eliminating endogenous TRAC expression.

23. The method of claim 21 or 22, wherein the mesothelin is expressed by a cancer cell.

24. The method of claim 23, wherein the cancer cell is a pancreatic, ovarian, lung, or breast cancer cell.

25. A method of making an engineered T cell receptor comprising a T cell receptor exchanged (Trex) locus, the method comprising
i) expressing a T cell receptor gene comprising a T cell receptor alpha (Trac) locus on a plasmid or vector containing homologous sequence to the murine Trac locus;
ii) inserting a donor TCR polynucleotide sequence into the T cell receptor alpha (Trac) locus of the T cell receptor gene using a nuclease-dependent cleavage system comprising Trac-specific targeting molecules specific to Trac exon 1 or directly 5′ to Trac exon 1 complexed to a ribonucleoprotein (RNP); and
iii) expressing the engineered TCR from the plasmid or vector.

26. A method of making an engineered T cell receptor comprising a T cell receptor exchanged (Trex) locus, the method comprising
i) expressing a T cell receptor gene comprising a T cell receptor alpha (Trac) locus on a plasmid or vector containing homologous sequence to the murine Trac locus;
i) inserting a donor TCR polynucleotide sequence into the T cell receptor alpha (Trac) locus of the T cell receptor gene using a CRISPR/Cas gene editing system comprising Trac-specific guide RNAs (gRNAs) specific to Trac exon 1 or directly 5′ to Trac exon 1 complexed to Cas ribonucleoprotein (RNP); and
iii) expressing the engineered TCR from the plasmid or vector.

27. The method of claim 26, wherein the donor TCR sequences comprise a TCRβ variable (V), TCRβ Constant (C) and TCRα V sequence.

28. The method of claim 27, wherein the exogenous TCRβ, TCRα, and endogenous Trac sequences are linked by self-cleaving 2A element.

29. The method of any one of claims 26 to 28, wherein the Cas comprises Cas9, Cas12a, Cas13a or Cas13b.

30. The method of any one of claims 26 to 29, wherein the guide RNAs are electroporated into activated splenic polyclonal T cells.

31. The method of any one of claims 25 to 30, wherein the donor TCR sequence is encoded in an AAV vector.

32. The method of any one of claims 25 to 31, wherein the donor TCR sequence is flanked by approximately 250 to 1000 bp homology arms (HA) encoding endogenous murine Trac sequences and cloned into an AAV vector.

33. The method of claim 31 or 32, wherein the AAV is AAV6, AAV1 or AAV-DJ.

34. The method of any one of claims 25 to 33, wherein CRISPR/Cas9 initiates a double-strand DNA break directly upstream of Trac or in exon 1.

35. The method of any one of claims 25 to 34, wherein T cells expressing a TRex TCR specific for the target antigen upon activation upregulate CD44 and maintain CD62L levels, downregulate TCR, and/or minimally express PD-1.

36. A method of making a genetically engineered non-human animal comprising a T cell receptor exchanged (Trex) locus, the method comprising
i) expressing a donor T cell receptor polynucleotide specific for a target antigen on a plasmid or vector; and
ii) inserting the donor TCR polynucleotide of i) into T cell receptor alpha (Trac) locus of a T cell receptor gene in a non-human animal zygote using a CRISPR/Cas gene editing system comprising Trac-specific guide RNAs (gRNAs) specific to Trac exon 1 (Trac gRNA 1) or directly 5′ to Trac exon 1 (Trac gRNA 2) complexed to a Cas ribonucleoprotein (RNP).

37. The method of claim 35, wherein the donor TCR sequences comprise a TCRβ variable (V), TCRβ Constant (C) and TCRα variable (V).

38. The method of claim 36, wherein the exogenous TCRβ, TCRα, and endogenous Trac sequences are linked by self-cleaving 2A element.

39. The method of any one of claims 36 to 38, wherein the guide RNAs are nucleofected into activated splenic polyclonal T cells.

40. The method of any one of claims 36 to 39, wherein the donor TCR sequence is expressed in an AAV vector.

41. The method of any one of claims 36 to 40, wherein the donor TCR sequence of interest is flanked by approximately 250-1000 bp homology arms (HA) encoding endogenous murine Trac sequences and cloned into rAAV.

42. The method of claim 40 or 41, wherein the AAV is AAV6, AAV1 or AAV-DJ.

43. The method of any one of claims 36 to 42, wherein CRISPR/Cas initiates a double-strand DNA break directly upstream of Trac or in exon 1.

44. The method of any one of claims 36 to 43, wherein rAAV expressing the TRex locus is administered to embryos at a final concentration of between 1.0×10⁸ GC/μl and 3×10⁸ GC/μl.

45. The method of any one of claims 36 to 44, further comprising inactivating a gene encoding the target antigen of interest in the non-human animal.

46. The method of claim 45, wherein the gene encoding the target antigen is inactivated using a nuclease-dependent cleavage system.

47. The method of claim 46, wherein the nuclease dependent cleavage system comprises a CRISPR/Cas system, a Cas-CLOVER system, a zinc-finger nuclease (ZFN) system, a transcription activator like effector nuclease (TALEN) system, or a meganuclease system

48. The method of claim 46 or 47 wherein the CRISPR/Cas system comprises Cas9, Cas12a, Cas13a or Cas13b.

49. The method of any one of claims 36 to 48, wherein the zygote is implanted into a pseudopregnant non-human animal.

50. The method of any one of claims 36 to 49, wherein 80% or more of CD4 and/or CD8 T cells in the genetically engineered non-human animal express an engineered TCR.

51. The method of any one of claims 36 to 49, wherein the T cells expressing the Trex TCR are not tolerized to the target antigen.

52. The method of any one of claims 36 to 51, wherein T cells expressing the Trex TCR upon activation upregulate CD44 and maintain CD62L levels, downregulate TCR, and/or minimally express PD-1.

53. The method of any one of claims 36 to 52, wherein the target antigen is mesothelin.

54. A genetically engineered non-human animal comprising
i) a TCR exchanged (TRex) T cell receptor locus expressing a T cell receptor specific for a protein of interest, wherein the non-human animal also expresses less than 15% endogenous T cell receptor on TCR α or β expressing cells; and
ii) an inactivated gene of the protein of interest.

55. The genetically engineered non-human animal of claim 54 wherein the TCR exchange is introduced in the T cell receptor alpha (Trac) locus.

56. The genetically engineered non-human animal of claim 54 or 55, wherein the TCR exchange comprises nuclease-dependent cleavage systemdisruption of the Trac locus and introduction of a polynucleotide encoding the T cell receptor specific for the protein of interest.

57. The genetically engineered animal of claim 56 wherein the nuclease dependent cleavage system comprises a CRISPR/Cas system, a Cas-CLOVER system, a zinc-finger nuclease (ZFN) system, a transcription activator like effector nuclease (TALEN) system, or a meganuclease system.

58. The genetically engineered animal of claim 56 wherein the CRISPR/Cas system comprises Cas9, Cas12a, Cas13a or Cas13b.

59. The genetically engineered non-human animal of any one of claims 54 to 58, wherein the polynucleotide encoding the T cell receptor specific for the protein of interest is expressed on a viral vector, optionally an AAV vector.

60. The genetically engineered non-human animal of any one of claims 54 to 59, wherein the animal expresses high affinity antigen-specific T cells.

61. The genetically engineered non-human animal of any one of claims 54 to 59, wherein the animal expresses low affinity antigen-specific T cells.

62. The genetically engineered non-human animal of any one of claims 54 to 61, wherein the T cells expressing the antigen-specific TCR are CD4+ T cells or CD8+ T cells.

61. The genetically engineered non-human animal of any one of claims 52 to 60, wherein the animal is a mouse.

62. The genetically engineered non-human animal of any one of claims 52 to 61, wherein the animal is homozygous for the donor TCR or heterozygous for the donor TCR.

63. The genetically engineered non-human animal of any one of claims 53 to 62 wherein the animal is homozygous for the protein knockout.

64. A T cell expressing a T cell receptor specific for a protein of interest isolated from a genetically engineered non-human animal of any one of claims 54 to 63.


SEQUENCE LISTING (20 sequences)

SEQ ID NO: 1 - DNA, 1812 bp, synthetic construct
  source 1..1812: mol_type = other DNA; note = Clone 1045 TCR
atgaccctgaagatggacagcagccccggcttcgtggccgtgatcctgctgatcctgggcagaacacacggcgacagcgtgacccagacagagggacctgtgaccgtgtccgagagcgagagcctgatcatcaactgcacctacagcgccacctctatcgcctaccccaacctgttttggtacgtgcgctaccccggcgagggactgcagctgctgctgaaagtgatcacagccggccagaagggcagcagcagaggcttcgaggccacctacaacaaagagacaaccagcttccatctgcaaaaggccagcgtgcaggaatccgacagcgccgtgtactactgtgccctgggcctggactacgccaacaagatgatcttcggcctgggcaccatcctgagagtgcggccccacatccagaaccctgagcctgctgtgtaccagctgaaggaccccagaagccaggacagcaccctgtgcctgttcaccgacttcgacagccagatcaacgtgcccaagaccatggaaagcggcaccttcatcaccgacaagaccgtgctggacatgaaggccatggactccaagagcaacggcgctatcgcctggtccaaccagaccagcttcacatgccaggacatcttcaaagagactaacgccacctaccccagcagcgacgtgccatgtgacgccaccctgaccgagaagtccttcgagacagacatgaacctgaacttccagaacctgtccgtgatgggcctgagaatcctgctgctgaaggtggccggcttcaacctgctgatgaccctgcggctgtggtccagcggttccggagccacgaacttctctctgttaaagcaagcaggagacgtggaagaaaaccccggtcccatggaccccaggctgctgtgctgcgtgatcttctgtctgctggccgccaccttcgtggacaccaccgtgaagcagaaccccagatacaagctggccagagtgggcaagcccgtgaacctgatctgcagccagaccatgaaccacgacaccatgtactggtatcagaagaagcccaaccaggcccccaaactgctgctgttctactacgacaagatcctgaacagagaggccgacacattcgagaagttccagagcagcagacccaacaacagcttctgcagcctgtacatcggctctgccggcctggaatactccgccatgtacctgtgcgccagcagcaaccctcaggacacccagtacttcggcccaggcaccagactgctggtgctggaagatctgagaaacgtgaccccccccaaggtgtccctgttcgagcctagcaaggccgagatcgctaacaagcagaaagccaccctcgtgtgtctggctagaggcttcttccccgaccacgtggaactgtcttggtgggtcaacggcaaagaggtgcacagcggcgtgtccacagacccccaggcctacaaagagtccaactacagctactgcctgagcagcaggctgagagtgtctgccaccttctggcacaaccccagaaaccacttcaggtgccaggtgcagttccacggcctgagcgaagaggacaagtggcctgagggcagccccaagcctgtgacacagaacatctctgccgaagcctggggcagggccgactgtggcattacatctgccagctaccaccagggcgtgctgagcgccacaatcctgtacgagattctgctgggcaaggccaccctgtacgccgtgctggtgtcaggcctggtgctgatggccatggtcaagaagaagaacagctgagtcgacgaattc

SEQ ID NO: 2 - DNA, 1818 bp, synthetic construct
  source 1..1818: mol_type = other DNA; note = Clone 7431 TCR
atgaccctgaagatggacagcagccctggcttcgtggccgtgatcctgctgatcctgggcagaacacacggcgacagcgtgacccagacagagggacctgtgaccgtgtccgagagcgagagcctgatcatcaactgcacctacagcgccacctctatcgcctaccccaacctgttttggtacgtgcgctaccccggcgagggactgcagctgctgctgaaagtgatcacagccggccagaagggcagcagcagaggcttcgaggctacctacaacaaagagacaaccagcttccatctgcaaaaggccagcgtgcaggaatccgacagcgccgtgtactactgtgccctgggccctgacaccaacgcctacaaagtgatcttcggcaagggcacccatctgcacgtgctgcccaacatccagaaccccgagcctgccgtgtaccagctgaaggaccctagaagccaggactccaccctgtgcctgttcaccgacttcgacagccagatcaacgtgcccaagaccatggaaagcggcaccttcatcaccgacaagaccgtgctggacatgaaggccatggactccaagagcaacggcgctatcgcctggtccaaccagaccagcttcacatgccaggacatcttcaaagagactaacgccacctaccccagcagcgacgtgccatgtgacgccaccctgaccgagaagtccttcgagacagacatgaacctgaacttccagaacctgagcgtgatgggcctgagaatcctgctgctgaaggtggccggcttcaacctgctgatgaccctgcggctgtggtcctctggttccggagccacgaacttctctctgttaaagcaagcaggagacgtggaagaaaaccccggtcccatggaccccagactgctgtgctgcgtgatcttctgtctgctggccgccaccttcgtggacaccaccgtgaagcagaaccccagatacaagctggccagagtgggcaagcccgtgaacctgatctgcagccagaccatgaaccacgacaccatgtactggtatcagaagaagcccaaccaggcccccaaactgctgctgttctactacgacaagatcctgaacagagaggccgacacattcgagaagttccagagcagcagacccaacaacagcttctgcagcctgtacatcggctctgccggcctggaatactccgccatgtacctgtgcgccagctccagaggcgccaacaccgaggtgttctttggcaagggaaccagactgaccgtggtggaagatctgagaaacgtgaccccccccaaggtgtccctgttcgagcctagcaaggccgagatcgccaacaagcagaaagccaccctcgtgtgtctggctagaggcttcttccccgaccacgtggaactgtcttggtgggtcaacggcaaagaggtgcacagcggcgtgtccacagacccccaggcttacaaagagtccaactacagctactgcctgagcagcaggctgagagtgtctgccaccttctggcacaaccccagaaaccacttcaggtgccaggtgcagttccacggcctgagcgaagaggacaagtggcctgagggcagccccaagcctgtgacacagaacatctctgccgaggcctggggcagagctgactgtggcatcacatccgccagctaccagcagggcgtgctgagcgccacaatcctgtacgagattctgctgggcaaggccaccctgtacgccgtgctggtgtctaccctggtcgtgatggccatggtcaagagaaagaacagctgagtcgacgaattc

SEQ ID NO: 3 - protein, 7 aa, synthetic construct
RRKRGSG

SEQ ID NO: 4 - RNA, 20 nt, synthetic construct
  source 1..20: mol_type = other RNA
  modified_base 1: mod_base = OTHER; note = Uracil
  modified_base 2..6: mod_base = OTHER; note = Uracil
  modified_base 10: mod_base = OTHER; note = Uracil
  modified_base 13: mod_base = OTHER; note = Uracil
tcttttaactggtacacagc

SEQ ID NO: 5 - RNA, 20 nt, synthetic construct
  source 1..20: mol_type = other RNA
  modified_base 1..2: mod_base = OTHER; note = Uracil
  modified_base 4: mod_base = OTHER; note = Uracil
  modified_base 8..9: mod_base = OTHER; note = Uracil
  modified_base 11: mod_base = OTHER; note = Uracil
  modified_base 15: mod_base = OTHER; note = Uracil
  modified_base 17: mod_base = OTHER; note = Uracil
  modified_base 19: mod_base = OTHER; note = Uracil
ttctgggttctggatgtctg

SEQ ID NO: 6 - RNA, 20 nt, synthetic construct
  source 1..20: mol_type = other RNA
  modified_base 6: mod_base = OTHER; note = Uracil
  modified_base 8: mod_base = OTHER; note = Uracil
  modified_base 10: mod_base = OTHER; note = Uracil
  modified_base 15: mod_base = OTHER; note = Uracil
ggaggtatctgacctgagca

SEQ ID NO: 7 - RNA, 20 nt, synthetic construct
  source 1..20: mol_type = other RNA
  modified_base 17: mod_base = OTHER; note = Uracil
  modified_base 19: mod_base = OTHER; note = Uracil
ggccaagaaagaggcctgtg

SEQ ID NO: 8 - DNA, 23 bp, synthetic construct
  source 1..23: mol_type = other DNA
gctagatcctaggctgtcatttc

SEQ ID NO: 9 - DNA, 22 bp, synthetic construct
  source 1..22: mol_type = other DNA
ccaatgtcctctgtcatgttct

SEQ ID NO: 10 - DNA, 20 bp, synthetic construct
  source 1..20: mol_type = other DNA
aggtgggttcagtacctttg

SEQ ID NO: 11 - DNA, 22 bp, synthetic construct
  source 1..22: mol_type = other DNA
gatcagctcagacttgggatag

SEQ ID NO: 12 - DNA, 20 bp, synthetic construct
  source 1..20: mol_type = other DNA
ctctggtgtgagtgctattc

SEQ ID NO: 13 - DNA, 22 bp, synthetic construct
  source 1..22: mol_type = other DNA
cctgttctggtacgtgagatac

SEQ ID NO: 14 - DNA, 23 bp, synthetic construct
  source 1..23: mol_type = other DNA
gtagctatgaggatagcaccttt

SEQ ID NO: 15 - protein, 9 aa, synthetic construct
GQKMNAQAI

SEQ ID NO: 16 - protein, 9 aa, synthetic construct
KAVYNFATM

SEQ ID NO: 17 - DNA, 66 bp, synthetic construct
  source 1..66: mol_type = other DNA
tcctcttggcccacagacatccagaacccagaacctgctgtgtaccagttaaaagatcctcggtct

SEQ ID NO: 18 - DNA, 76 bp, synthetic construct
  source 1..76: mol_type = other DNA
ttggcttgttccatgctcaggtcagatacctcctcacatgtgaggccaagaaagaggcctgtggggagactgggga

SEQ ID NO: 19 - DNA, 46 bp, synthetic construct
  source 1..46: mol_type = other DNA
tcctcttggcccacagacatccagaacccagaacctgctgtgtacc

SEQ ID NO: 20 - DNA, 42 bp, synthetic construct
  source 1..42: mol_type = other DNA
tcacatgtgaggccaagaaagaggcctgtggggagactggga